(19) United States
(12) Patent Application Publication
(10) Pub. No.: US 2009/0130774 A1
(43) Pub. Date: May 21, 2009

(54) Elisa assays using prion-specific peptide reagents

(76) Inventors: David Peretz, El Cerrito, CA (US); Melissa Michelitsch, Berkeley, CA (US); Celine Hu, Tiburon, CA (US); Xuemei Wang, Castro Valley, CA (US); Man Gao, San Ramon, CA (US); Michael Connolly, Hercules, CA (US); Thomas Horn, Berkeley, CA (US); Ronald Zuckermann, El Cerrito, CA (US); David Chien, Alamo, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 11/795,163

(22) PCT Filed: Jan. 13, 2006
(86) PCT No.: PCT/US06/01437

§ 371 Date: Jan. 20, 2009

Related U.S. Application Data
(60) Provisional application No. 60/644,314, filed on Jan. 13, 2005.
(60) Provisional application No. 60/652,493, filed on Feb. 11, 2005.

(51) Int. Cl. G01N 33/566

(52) U.S. Cl. 436/501

(57) ABSTRACT

Peptide reagents that interact preferentially with the PrPsc form of the prion protein are described for use in detecting PrPsc in biological samples. In particular, ELISA assays are described.

### FIELD OF THE INVENTION

The invention relates to peptide reagents that interact with prion proteins, polynucleotides encoding these peptide reagents, methods of generating antibodies using such peptide reagents and polynucleotides, and to antibodies generated using these methods. The invention further relates to methods of using these peptide reagents to detect the presence of pathogenic prions in a sample and to methods of using these peptide reagents as components in a therapeutic or prophylactic composition.

### BACKGROUND

Protein conformational diseases include a variety of unrelated diseases, including transmissible spongiform encephalopathies, arising from aberrant conformational transition of a protein (a conformational disease protein) which in turn leads to self-association of the aberrant protein forms, with consequent tissue deposition and damage. These diseases also share striking similarities in clinical presentations, typically a rapid progression from diagnosis to death following varying lengths of incubation.

One group of confomational diseases is termed “prion diseases” or “transmissible spongiform encephalopathies (TSEs).” In humans these diseases include Creutzfeldt-Jakob disease (CJD), Gerstmann-Straussler-Scheinker syndrome (GSS), Fatal Familial Insomnia, and Kuru (see, e.g., Harrison's Principles of Internal Medicine, Isselbacher et al., eds., McGraw-Hill, Inc. New York, (1994); Medori et al. (1992) N. Engl. J. Med. 326: 444-9.). In animals the TSE's include sheep scrapie, bovine spongiform encephalopathy (BSE), transmissible mink encephalopathy, and chronic wasting disease of captive mule deer and elk (Gajdusek, (1990) Subacute Spongiform Encephalopathies: Transmissible Cerebral Amyloidoses Caused by Unconventional Viruses. Pp. 2289-2324 In: Virology, Fields, ed. New York: Raven Press, Ltd.). Transmissible spongiform encephalopathies are characterized by the same hallmarks: the presence of the abnormal (beta-rich, proteinase K resistant) conformation of the prion protein that transmits disease when experimentally inoculated into laboratory animals including primates, rodents, and transgenic mice.

Recently, the rapid spread of bovine spongiform encephalopathy and its correlation with elevated occurrence of spongiform encephalopathies in humans has lead to a significant increase of interest in the detection of transmissible spongiform encephalopathies in non-human mammals. The tragic consequences of accidental transmission of these diseases (see, e.g., Gajdusek, Infectious Amyloids, and Prusiner Prions In Fields Virology. Fields, et al., eds. Lippincott-Ravin, Pub. Philadelphia (1996); Brown et al. (1992) Lancet, 340: 24-27), decontamination difficulties (Asher et al. (1986) pages 59-71 In: Laboratory Safety: Principles and Practices, Miller ed. Am. Soc. Microb.), and recent concern about bovine spongiform encephalopathy (British Med. J. (1995) 311: 1415-1421) underlie the urgency of having both a diagnostic test that would identify humans and animals with transmissible spongiform encephalopathies and therapies for infected subjects.

Prions are the infectious pathogen that causes spongiform encephalopathies (prion diseases). Prions differ significantly from bacteria, viruses and viroids. The dominating hypothesis is that, unlike all other infectious pathogens, infection is caused by an abnormal conformation of the prion protein, which acts as a template and converts normal prion conformations into abnormal conformations. A prion protein was first characterized in the early 1980s. (See, e.g., Bolton, McKinley et al. (1982) Science 218:1309-1311; Prusiner, Bolton et al. (1982) Biochemistry 21:6942-6950; McKinley, Bolton et al. (1983) Cell 35:57-62). Complete prion protein-encoding genes have since been cloned, sequenced and expressed in transgenic animals. See, e.g., Basler, Oesch et al. (1986) Cell 46:417-428.

The key characteristic of prion diseases is the formation of an abnormally shaped protein (PrP^{Sc}), also referred to as a scrapie protein, from the normal (cellular or nonpathogenic) form of prion protein (PrP^{C}). See, e.g., Zhang et al. (1997) Biochem. 36(12):3543-3553; Cohen & Prusiner (1998) Ann Rev. Biochem. 67:793-819; Pan et al. (1993) Proc Nat'l Acad Sci USA 90:10962-10966; Safar et al. (1993) J Biol Chem 268:20276-20284. Optical spectroscopy and crystallography studies have revealed that disease-related forms of prions are substantially enriched in beta-sheet structure as compared to the predominantly alpha-helical folded non-disease forms. See, e.g., Wille et al. (2001) Proc. Nat'l Acad. Sci. USA 99:3563-3568; Peretz et al. (1997) J. Mol. Biol. 273:614-622; Cohen & Prusiner, Chapter 5: Structural Studies of Prion Proteins in PRION BIOLOGY AND DISEASES, ed. S. Prusiner, Cold Spring Harbor Laboratory Press, 1999, pp: 191-228). The structural changes appear to be followed by alterations in the biochemical properties: PrP^{C} is soluble in non-denaturing detergents, PrP^{Sc} is insoluble; PrP^{C} is readily digested by proteases, while PrP^{Sc} is partially resistant, resulting in the formation of an N-terminally truncated fragment known as “PrPres” (Baldwin et al. (1995); Cohen & Prusiner (1995); Safar et al. (1998) Nat. Med. 4(10):1157-1165), “PrP 27-30” (27-30 kDa) or “PK-resistant” (proteinase K resistant) form. In addition, PrP^{Sc} can convert PrP^{C} to the pathogenic conformation. See, e.g., Kaneko et al. (1995) Proc. Nat'l Acad. Sci. USA 92:11160-11164; Caughey (2003) Br Med. Bull. 66:109-20.

Detection of the pathogenic isoforms of conformational disease proteins in living subjects and samples obtained from living subjects has proven difficult. Thus, definitive diagnosis and palliative treatments for these transmissible and amyloid containing conditions before death of the subject remains a substantially unmet challenge. Histopathological examination of brain biopsies is risky to the subject and lesions and amyloid deposits can be missed depending on where the biopsy sample is taken from. However, there are still risks involved with biopsies to animals, patients, and health care personnel. Further, the results from brain tests on animals are not usually obtained until the animal has entered the food supply. In addition, most antibodies generated against prion peptides recognize both denatured PrP^{Sc} and PrP^{C} although there have been reports of antibodies that are specific for native PrP^{Sc}. (See, e.g., Matsunaga et al. (2001) PROTEINS: Structure, Function and Genetics 44:110-118; U.S. Pat. Nos. 5,846,533 and 6,765,088).

A number of tests for TSE are available (See, Soto, C. (2004) Nature Reviews Microbiol. 2:809, Biffiger et al. (2002) J. Virol. Meth. 101:79; Safar et al. (2002) Nature Biotech. 20:1147, Schaller et al. Acta Neuropathol. (1999) 98:437, Lane et al. (2003) Clin. Chem. 49:1774). However, all of these utilize brain tissue samples and are suitable only as post-mortem tests. Most of these require proteinase K treatment of the samples as well which can be time-consuming, incomplete digestion of the PrP^{C} can lead to false positive results, and digestion of protease-sensitive PrP^{SC} may lead to false negative results.

Thus, there remains a need for compositions and methods for detecting the presence of pathogenic prion proteins in various samples, for example in samples obtained from living subjects, in blood supplies, in farm animals and in other human and animal food supplies. In addition, there remains a need for methods and compositions for diagnosing and treating prion-related diseases

### SUMMARY OF THE INVENTION

The present inventors have developed a sensitive method for detection of pathogenic prion proteins. The method is sufficiently sensitive to detect low levels of pathogenic prions that may be present in the biological fluids of individuals afflicted with a prion-related disease. The method is thus useful, inter alia, as an ante-mortem diagnostic test or for screening donated blood samples.

The present invention relates, in part, to peptide reagents that interact with prion proteins. More specifically, the peptide reagents interact preferentially with the pathogenic isoforms of prion proteins. Such peptide reagents have been described in co-owned patent applications U.S. Ser. No. 10/917,646, filed Aug. 13, 2004; U.S. Ser. No. 11/056,950, filed Feb. 11, 2005; and PCT application No. PCT/US2004/026363, filed Aug. 13, 2004, all of which applications are incorporated by reference herein. The peptide reagents are used to concentrate and separate pathogenic prion protein in test samples. Unlike previously described assays for the PrP^{Sc} the present method does not require any protease treatment of the samples to remove PrP^{C}. In the method of the present invention, the peptide reagents are used in combination with a sensitive ELISA for detection of the concentrated and separated prion protein.

In one embodiment the invention provides a method for detecting the presence of a pathogenic prion in a sample comprising:

(a) providing a first solid support comprising a peptide reagent that interacts preferentially with the pathogenic form of a prion;

(b) contacting the first solid support with a sample under conditions that allow pathogenic prion proteins, when present in the sample, to bind to the peptide reagent;

(c) removing unbound sample;

(d) dissociating the pathogenic prion proteins from the peptide reagent; and

(e) detecting the dissociated pathogenic prions using a prion-binding reagent.

The peptide reagents are preferably derived from a peptide having a sequence selected from the group consisting of SEQ ID NO:12-260 and are described in detail in co-owned patent applications U.S. Ser. No. 10/917,646, filed Aug. 13, 2004; U.S. Ser. No. 11/056,950, filed Feb. 11, 2005; and PCT application No. PCT/US2004/026363, filed Aug. 13, 2004. After removal of any unbound sample, the pathogenic prion protein is dissociated from the peptide reagent. Typically, the pathogenic prion protein is denatured in the process of dissociation. The dissociation is accomplished through the use of a chaotropic agent (for example, guanidinium thiocyanate or guanidinium HCl) or high salt concentrations or, preferably, by changing the pH. Either low pH (e.g, below pH 2) and high pH (above pH 12) can be used, although high pH is preferred. The dissociated and denatured prion protein is detected using an immunoassay, preferably an ELISA, more preferably a sandwich ELISA, using anti-prion antibodies.

The invention also provides kits for carrying out the method, which kits include one or more peptide reagents, which peptide reagents may be provided on a solid support, and optionally, one or more anti-prion antibodies. The anti-prion antibodies may be labeled and/or may be provided on a solid support. Buffers, wash solutions, denaturants and other components used in the method may optionally be included in the kit, as are instructions for use.

These peptide reagents can be used in a wide range of applications, including as tools to isolate pathogenic prions or to detect the presence of pathogenic prions in a sample, as components of a therapeutic or prophylactic composition and/or to generate prion-specific antibodies. For example, peptide reagents that interact preferentially with PrP^{Sc} as compared to PrP^{C} are useful for direct detection of pathogenic forms in samples obtained from living subjects, for example, for diagnosis of a disease or for screening donated blood samples or screening organs for organ donation.

The peptide reagents described herein may be partially or fully synthetic, for example, may comprise one or more the following moieties: cyclized residues or peptides, multimers of peptides, labels, and/or other chemical moieties. Examples of suitable peptide reagents include those derived from peptides of SEQ ID NOs:12-260, for example, peptides such as those depicted in SEQ ID NOs: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 133, 134, 135, 136, 56, 57, 65, 82, or 84, and analogs and derivatives thereof. The peptide reagents described herein may interact with any conformational disease proteins, for example, prion proteins (e.g., the pathogenic protein PrP^{Sc}, and the nonpathogenic form PrP^{C}). In certain embodiments, peptide reagents interact preferentially with PrP^{Sc} as compared to PrP^{C}. The peptide reagents will generally be specific for PrP^{Sc} from more than one species, but may be specific for PrP^{Sc} from a single species.

In another embodiment, peptide reagents derived from peptides shown in any of sequences described herein are provided. In certain embodiments, the peptide reagents are derived from regions of a prion protein, for example, those regions corresponding to residues 23-43 or 85-156 (e.g., 23-30, 86-111, 89-112, 97-107, 113-135, and 136-156 numbered according to the mouse prion sequence shown in SEQ ID NO:2) are employed. For convenience, the amino acid residue numbers set out above are those corresponding to the mouse prion protein sequence in SEQ ID NO:2; one of ordinary skill in the art could readily identify corresponding regions in prion proteins of other species based on the sequences known in the art and the teachings provided herein. Exemplary peptide reagents include those derived from peptides having SEQ ID NO:66, 67, 68, 72, 81, 96, 97, 98, 107, 108, 119, 120, 121, 122, 123, 124, 125, 126, 127, 134 or 135; or from peptides having SEQ ID NO:14, 35, 36, 37, 40, 50, 51, 77, 89, 100, 101, 109, 110, 111, 112, 113, 114, 115, 116, 117, 118, 129, 130, 131, 132, 133 or 128; or from peptides having SEQ ID NO:56, 57, 65, 82, 84, or 136.

In one aspect, methods for detecting the presence of prion proteins are provided. The detection methods may be used, inter alia, in connection with methods for diagnosing a prion-related disease (e.g., in human or non-human animal subjects), ensuring a substantially PrP^{Sc}-free blood supply, blood products supply, or food supply, analyzing organ and tissue samples for transplantation, monitoring the decontamination of surgical tools and equipment, as well as any other situation in which knowledge of the presence or absence of the pathogenic prion is important.

The detection methods rely on the preferential interaction of the peptide reagents with the pathogenic prion isoform. In certain embodiments, a method for detecting the presence of a pathogenic prion in a biological sample is provided.

In one embodiment, the method comprises contacting the sample suspected of containing a pathogenic prion with one or more of the peptide reagents described herein under conditions that allow the interaction of the peptide reagent(s) and the pathogenic prion, if present; and detecting the presence or absence of the pathogenic prion in the sample by its binding to the peptide reagent(s). The interaction of the peptide reagent(s) and the pathogenic prion can be carried out in solution, or one or more of the reactants can be provided in or on a solid phase. Sandwich-type assays can be carried out in which the peptide reagents can be used as a capture reagent, a detection reagent or both. In preferred embodiments, other prion-binding reagents (e.g., antibodies and other binding molecules that bind to denatured prion protein) may be used in this aspect in combination with the peptide reagents of the invention.

In one aspect of this embodiment, one or more peptide reagents of the present invention is provided on a solid support and contacted with a sample suspected of containing a pathogenic prion, under conditions that allow binding of the pathogenic prion, if present, to the peptide reagent. Unbound sample materials, including any non-pathogenic prion, can be removed and the pathogenic prion can be detected, either while remaining bound to the peptide reagent or after dissociation from the peptide reagent. The pathogenic prion can be detected using a detectably labeled peptide reagent (either the same peptide reagent used to “capture” the pathogenic prion or a second peptide reagent of the invention) or a detectably labeled anti-prion antibody or other prion-binding reagent. This antibody or prion-binding reagent need not be specific for the pathogenic form of the prion.

In a further aspect of this embodiment, the pathogenic prion is dissociated from the peptide reagent, denatured and detected using a sandwich-type assay with anti-prion antibodies.

In a further embodiment, the method comprises contacting the sample suspected of containing a pathogenic prion with one or more peptide reagents selected from the group consisting of peptides having the sequences of SEQ ID NO:12-260, and analogs and derivatives thereof, under conditions which allow the binding of the peptide reagent(s) to the pathogenic prion, if present; and detecting the presence or absence of the pathogenic prion in the sample by its binding to the peptide reagent(s). In preferred embodiments, the sample is contacted with one or more peptide reagents selected from the group consisting of peptides having the sequences of SEQ ID NO:66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 133, 134, 135, 56, 57, 65, 82, 136 or 84, and analogs and derivatives thereof.

Any of the above methods of detection of a pathogenic prion can be used in a method to diagnose a prion-related disease.

In all of the foregoing embodiments providing a solid support comprising one or more peptide reagents of the invention, alternative embodiments are contemplated in which the peptide reagent is contacted with the sample prior to the peptide reagent being attached to the solid support. In these embodiments, the peptide reagent comprises one member of a binding pair and the solid support comprises the second member of the binding pair. For example, the peptide reagent of the invention may contain or be modified to contain biotin. The biotinylated peptide reagent is contacted with a sample suspected to contain a pathogenic prion under conditions to allow binding of the peptide reagent to the pathogenic prion. A solid support comprising avidin or streptavidin is then contacted with the biotinylated peptide reagent. Other suitable binding pairs are described herein.

In any of the methods using a solid support described herein, the solid support can be, for example, nitrocellulose, polystyrene, polypropylene, latex, polyvinyl fluoride, diazotized paper, nylon membranes, activated beads, and/or magnetically responsive beads, polyvinylchloride; polypropylene, polystyrene latex, polycarbonate, nylon, dextran, chitin, sand, silica, pumice, agarose, cellulose, glass, metal, polyacrylamide, silicon, rubber, polysaccharides, diazotized paper; activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads or particles, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer). The terms “solid support” and “solid surface” are used interchangeably herein.

In addition, in any of the methods described herein the sample can be a biological sample, that is, a sample obtained or derived from a living or once-living organism, for example, organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. In preferred embodiments, the biological sample comprises blood, blood fractions or blood components. The sample may be a non-biological sample.

In another aspect, the present invention provides a method of diagnosing a prion-related disease in a subject by detecting the presence of a pathogenic prion in a biological sample from said subject by any of the detection methods described herein.

In another aspect, the invention includes methods of preparing a blood supply that is substantially free of pathogenic prions, the method comprising the steps of screening aliquots of blood (e.g., whole blood, plasma, platelets or serum) from collected blood samples by any of the methods described herein; eliminating any sample in which pathogenic prions are detected; and combining samples where pathogenic prions are not detected to provide a blood supply substantially free of pathogenic prions.

In yet another aspect, the invention includes methods of preparing a food supply, in particular, a meat supply (e.g., beef, lamb, mutton or pork used for human or animal consumption) that is substantially free of pathogenic prions, the method of comprising the steps of screening, using any of the methods of detection described herein, samples collected from live or dead organisms that will enter the food supply or samples collected from food intended to enter the food supply; identifying samples in which pathogenic prions are detected; and removing from the food supply any live or dead organism or food intended to enter the food supply, in samples from which, pathogenic prions are detected; thereby providing a food supply that is substantially free of pathogenic prions.

In another aspect, the invention includes various kits for detecting the presence of a pathogenic prion in a sample, for isolating a pathogenic prion from a sample, for eliminating a pathogenic prion from a sample, the kit comprising: one or more of the peptide reagents described herein; and/or any of the solid supports comprising one or more of the peptide reagents described herein, anti-prion antibodies and other necessary reagents and, optionally, positive and negative controls and/or surrogate positive controls. The invention also provides molecules useful as surrogate positive control for the assays described herein.

These and other embodiments of the subject invention will readily occur to those of skill in the art in light of the disclosure herein.

### BRIEF DESCRIPTION OF THE DRAWINGS

FIG. 1 depicts the amino acid sequence of human (SEQ ID NO:1) and mouse (SEQ ID NO:2) prion proteins.

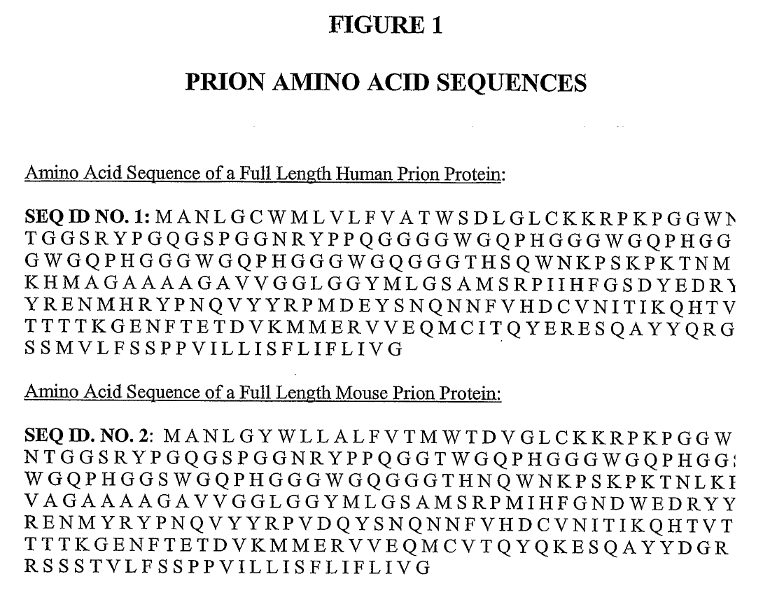

FIG. 2 depicts an alignment of prion proteins from human (SEQ ID NO:3), Syrian hamster (hamster) (SEQ ID NO:4), bovine (SEQ ID NO:5), sheep (SEQ ID NO:6), mouse (SEQ ID NO:7), elk (SEQ ID NO:8), fallow deer (fallow) (SEQ ID NO:9), mule deer (mule) (SEQ ID NO:10), and white tailed deer (white) (SEQ ID NO:11). Elk, Fallow Deer, Mule Deer, and White Tailed Deer only vary from each other at two residues, S/N128 and Q/E226 (shown in bold).

FIG. 3, panels A-F depict exemplary peptoid substitutions that may be made to prepare any of the peptide reagents described herein. The peptoids are circled in each panel and are shown in an exemplary peptide reagent as described herein (SEQ ID NO:14, QWNXPSKPKTN), in which a proline residue (residue 8 of SEQ ID NO:14) is replaced with an N-substituted glycine (peptoid) residue. Panel A shows a peptide reagent in which a proline residue is substituted with the peptoid residue: N-(S)-(1-phenylethyl)glycine; panel B shows a peptide reagent in which a proline residue is substituted with the peptoid residue: N-(4-hydroxyphenyl)glycine; panel C shows a peptide reagent in which a proline residue is substituted with the peptoid residue: N-(cyclopropylmethyl)glycine; panel D shows a peptide reagent in which a proline residue is substituted with the peptoid residue: N-(isopropyl)glycine; panel E shows a peptide reagent in which a proline residue is substituted with the peptoid residue: N-(3,5-dimethoxybenzyl)glycine; and panel F shows a peptide reagent in which a proline residue is substituted with the peptoid residue: N-amino butylglycine.

FIG. 4 depicts results of Western blotting experiments as described in Example 2. Lanes 1 and 2 show the presence of prion proteins in normal mouse brain homogenates (Lane 1, labeled “C”) and in denatured infected mouse brain homogenates (lane 2, labeled “Sc”). Lanes 3, 4 and 5 show specific binding of a peptide reagent as described herein (SEQ ID NO:68) to pathogenic prion forms in the presence of human plasma. In particular, Lane 3 is a human plasma control and lane 4 is a normal mouse brain homogenate sample. Lane 5 shows strong binding by the peptide reagent to PrPSc in infected mouse brain homogenate samples.

FIG. 5 depicts the structures of exemplary PEG-linked peptide reagents as described herein.

FIG. 6 depicts the structure of (QWNKPSKPKTN)2K (SEQ ID NO:133).

FIG. 7, panels A to C depict an exemplary PrP^{Sc} detection assay. FIG. 7A shows capture of PrP^{Sc} using magnetic beads coated with a PrP^{Sc}-specific peptide reagent as described herein. The beads and bound PrP^{Sc} are pulled down in a magnetic field and washed. FIG. 7B shows elution, denaturing of PrP^{Sc} and coating of the denatured PrPsc to the well for ELISA. FIG. 7C shows detection of the PrP^{Sc} coated to the wells by two-antibody ELISA.

FIG. 8 is a graph depicting ELISA detection of mouse PrP^{Sc} brain homogenate at various dilutions in normal mouse brain homogenates.

FIG. 9, panels A and B depict ELISA detection of mouse PrP^{Sc} spiked into human plasma samples. FIG. 9A depicts ELISA detection with QWNKPSKPKTN-biotin (SEQ ID NO:14). FIG. 9B depicts ELISA detection with biotin-GGGKRPKPGG (SEQ ID NO:68).

FIG. 10, panels A and B, depict ELISA and Western Blot detection, respectively, of FIG. 10A depicts ELISA detection of PrP^{Sc} in normal and scrapie infected Syrian hamsters (SHa). FIG. 11A depicts ELISA detection of pulled-down PrPsc without Proteinase K-digestion using QWNKPSKPKTN-biotin (SEQ ID NO:14) (dark bars) or biotin-GGGKRPKPGG (SEQ ID NO:68) (white bars). FIG. 10B depicts Western blot analysis of PK-digested samples. “MW” refers to molecular weight. Lanes 1 and 2 show analysis of two different samples of normal SHa brain homogenates. Lanes 3 and 4 show analysis of two different samples of PrP^{Sc} SHa brain homogenates. Lane 5 shows analysis of normal mouse brain homogenates. Lane 6 shows analysis of PrP^{sc} mouse brain homogenates.

FIG. 11 is a graph depicting ELISA results on samples obtained from normal and infected mice transgenic for the deer PrP gene. PrP^{sc} was pulled down using QWNKPSKPKTN-biotin (SEQ ID NO:14) (black and light gray rectangles), biotin-KKKAGAAAAGAVVGLGG-CONH2 (SEQ ID NO:136) (light gray rectangles), and GGGKRPKPGG (SEQ ID NO:68) (dark gray rectangles) and detected by ELISA.

FIG. 12, panels A and B, depict Western Blot and ELISA detection, respectively, of FIG. 12A depicts Western Blot analysis detection of CJD (sCJD, vCJD, infected SHa). FIG. 12B depicts ELISA detection of pulled-down CJD with Proteinase K-digestion.

FIG. 13 is a graph depicting ELISA detection of PrP^{Sc} from human vCJD brain homogenates using various peptide reagents as described herein. Prion-specific reagents are as follows: QWNKPSKPKTN-biotin (SEQ ID NO:14); QWNKPSKPTKTNGGGQWNKPSKPKTN-biotin (SEQ ID NO:51); biotin-QWNKPSKPKTN, where P5 is substituted with N-(3,5-dimethoxybenzyl)glycine (SEQ ID NO:117); biotin-QWNKPSKPKTN, where P5 is substituted with N-amino butylglycine (SEQ ID NO:118); biotin-QWNKPSKPKTN, where P8 is substituted with N-(cyclopropylmethyl)glycine (SEQ ID NO:111); biotin-QWNKPSKPKTN, where P8 is substituted with N-amino butylglycine (SEQ ID NO:114); biotin-QWNKPSKPKTN, where P5 is substituted with N-(cyclopropylmethyl)glycine and P8 is substituted with N-amino butylglycine (SEQ ID NO:131); biotin-QWNKPSKPKTN, where P5 is substituted with N-(isopropyl)glycine and P8 is substituted with N-(cyclopropylmethyl)glycine (SEQ ID NO:132); QWNKPSKPKTN2K-biotin (SEQ ID NO:133); biotin-GGGKKRPKPGG (SEQ ID NO:68); biotin-KKRPKPGG, where P6 is substituted with N-(cyclopropylmethyl)glycine (SEQ ID NO:122); biotin-GGGKKRPKPGGGQWNKPSKPKTN (SEQ ID NO:81); 4-branchMAPS-GGGKKRPKPGGWNTGGG-biotin (SEQ ID NO:134); 8-branchMAPS-GGGKKRPKPGGWNTGGG-biotin (SEQ ID NO:135); biotin-KKKAGAAAAGAVVGGLGGYMLGSAM (SEQ ID NO:57); biotin-KKKAGAAAAGAVVGGLGG-CONH2 (SEQ ID NO:136); and biotin-GGGKKKKKKKK (SEQ ID NO:85).

FIG. 14 depicts detection when the peptide reagent is coated onto the bead prior to incubation with the sample suspected of containing a pathogenic prion as compared to detection when the peptide reagent is coated onto the bead after incubation with the sample. Pre-coated (black circles) was approximately 100 times more efficient at detection than post-incubation coating (white circles).

### DETAILED DESCRIPTION

The present invention provides a method of detection for pathogenic prion proteins that combines the use of peptide reagents that interact preferentially with the pathogenic prion proteins (as compared to the non-pathogenic prion proteins) together with an improved ELISA procedure.

The invention relates to the surprising and unexpected discovery that relatively small peptides (less than 50 to 100 amino acids in length, preferably less than 50 amino acids in length and even more preferably less than about 30 amino acids in length) can be used to discriminate between nonpathogenic and pathogenic prion proteins. Thus, the present disclosure relates to the surprising finding that these peptides and derivatives thereof (collectively “peptide reagents”), may bind pathogenic and nonpathogenic protein forms at different specificity and/or affinity and, accordingly, can be used, in and of themselves, as diagnostic/detection reagents or as components of therapeutic compositions. Prior to the present disclosure, it was believed that only larger molecules (e.g., antibodies, PrP^{C}, α-form rPrP and plasminogen) could be used to differentiate pathogenic and nonpathogenic forms. As such, previously described antigenic peptides were used to generate antibodies that were evaluated for their ability to discriminate between pathogenic and nonpathogenic forms. However, due to the relatively nonimmunogenic nature of prion proteins, it has proven difficult to generate antibodies specific for pathogenic forms. See, e.g., R. A. Williamson et al. “Antibodies as Tools to Probe Prion Protein Biology” in PRION BIOLOGY AND DISEASES, ed. S. Prusiner, Cold Spring Harbor Laboratory Press, 1999, pp: 717-741.

The discovery that certain peptides as described herein interact preferentially with pathogenic (PrP^{Sc}) prion proteins allows for the development of novel reagents for diagnostics, detection assays and therapeutics, inter alia. Thus, the invention relates to peptide reagents and, in addition, relates to detection assays and diagnostic assays utilizing these peptide reagents, purification or isolation methods utilizing these peptide reagents and therapeutic compositions comprising these peptide reagents. Also provided are polynucleotides encoding these peptide reagents, and antibodies generated using these peptide reagents. The peptide reagents, polynucleotides and/or antibodies described herein are useful in compositions and methods for detecting the presence of pathogenic prions, for example in a biological sample. In addition, the invention further relates to methods of using such peptide reagents, antibodies and/or polynucleotides as a component in a therapeutic or prophylactic composition.

The peptide reagents used in the invention comprise a peptide that interacts preferentially with pathogenic isoforms as compared to nonpathogenic isoforms. For example, in certain embodiments, peptide reagents as described herein specifically bind to pathogenic conformational disease protein forms and do not bind (or bind to a lesser extent) to non-pathogenic forms. The peptide reagents described herein may be used, for example, to generate antibodies. These antibodies may recognize pathogenic forms, non-pathogenic forms or both. These molecules are useful, alone or in various combinations, in diagnostic assays and/or in prophylactic or therapeutic compositions.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods in Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B.N. Raven Press, New York, N.Y.

It is understood that the peptide reagents, antibodies and methods of this invention are not limited to particular formulations or process parameters as such may, of course, vary. It is also to be understood that the terminology used herein is for the purpose of describing particular embodiments of the invention only, and is not intended to be limiting.

All publications, patents and patent applications cited herein are hereby incorporated by reference in their entirety.

### I. DEFINITIONS

In order to facilitate an understanding of the invention, selected terms used in the application will be discussed below.

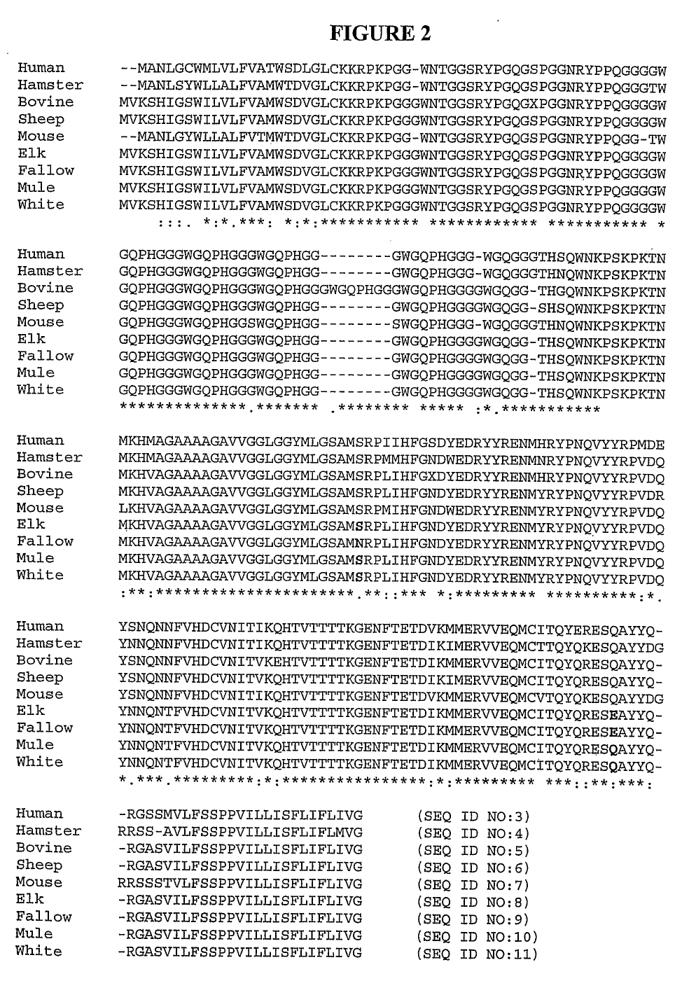

The terms “prion”, “prion protein”, “PrP protein” and “PrP” are used interchangeably herein to refer to both the pathogenic protein form (variously referred to as scrapie protein, pathogenic protein form, pathogenic isoform, pathogenic prion and PrP^{Sc}) and the non-pathogenic form (variously referred to as cellular protein form, cellular isoform, nonpathogenic isoform, nonpathogenic prion protein, and PrP^{C}), as well as the denatured form and various recombinant forms of the prion protein which may not have either the pathogenic conformation or the normal cellular conformation. The pathogenic protein form is associated with disease state (spongiform encephalopathies) in humans and animals; the non-pathogenic form is normally present in animal cells and may, under appropriate conditions, be converted to the pathogenic PrP^{Sc} conformation. Prions are naturally produced in a wide variety of mammalian species, including human, sheep, cattle, and mice. A representative amino acid sequence of a human prion protein is set forth as SEQ ID NO:1. A representative amino acid sequence of a mouse prion protein is set forth as SEQ ID NO:2. Other representative sequences are shown in FIG. 2.

As used herein, the term “pathogenic” may mean that the protein actually causes the disease or it may simply mean that the protein is associated with the disease and therefore is present when the disease is present. Thus, a pathogenic protein as used in connection with this disclosure is not necessarily a protein that is the specific causative agent of a disease. Pathogenic forms may or may not be infectious. The term “pathogenic prion form” is used more specifically to refer to the conformation and/or the beta-sheet-rich conformation of mammalian, avian or recombinant prion proteins. Generally, the beta-sheet-rich conformation is proteinase K resistant. The terms “non-pathogenic” and “cellular” when used with respect to conformational disease protein forms are used interchangeably to refer to the normal isoform of the protein whose presence is not associated with sickness.

Furthermore, a “prion protein” or “conformational disease protein” as used herein is not limited to a polypeptide having the exact sequence to those described herein. It is readily apparent that the terms encompass conformational disease proteins from any of the identified or unidentified species or diseases (e.g., Alzheimer's, Parkinson's, etc.). One of ordinary skill in the art in view of the teachings of the present disclosure and the art can determine regions corresponding to the sequences shown in the Figures in any other prion proteins, using for example, sequence comparison programs (e.g., BLAST and others described herein) or identification and alignment of structural features or motifs.

The term “PrP gene” is used herein to describe any genetic material that expresses prion proteins including known polymorphisms and pathogenic mutations. The term “PrP gene” refers generally to any gene of any species that encodes any form of a PrP protein. Some commonly known PrP sequences are described in Gabriel et al., Proc. Natl. Acad. Sci. USA 89:9097-9101 (1992), and U.S. Pat. Nos. 5,565,186; 5,763,740; 5,792,901; and WO97/04814, incorporated herein by reference to disclose and describe such sequences. The PrP gene can be from any animal, including the “host” and “test” animals described herein and any and all polymorphisms and mutations thereof, it being recognized that the terms include other such PrP genes that are yet to be discovered. The protein expressed by such a gene can assume either a PrP^{C} (non-disease) or PrP^{sc} (disease) form.

“Prion-related disease” as used herein refers to a disease caused in whole or in part by a pathogenic prion protein (PrP^{Sc}). Prion-related diseases include, but are not limited to, scrapie, bovine spongiform encephalopathies (BSE), mad cow disease, feline spongiform encephalopathies, kuru, Creutzfeldt-Jakob Disease (CJD), new variant Creutzfeldt-Jakob Disease (nvCJD), chronic wasting disease (CWD), Gerstmann-Strassler-Scheinker Disease (GSS), and fatal familial insomnia (FFI).

The term “peptide reagent” as used herein generally refers to any compound comprising naturally occurring or synthetic polymers of amino acid or amino acid-like molecules, including but not limited to compounds comprising only amino and/or imino molecules. The peptide reagents of the present invention interact preferentially with a pathogenic prion protein and are typically derived from fragments of a prion protein. The term “peptide” will be used interchangeably with “oligopeptide” or “polypeptide” and no particular size is implied by use of these terms Included within the definition are, for example, peptides containing one or more analogs of an amino acid (including, for example, unnatural amino acids, peptoids, etc.), peptides with substituted linkages, as well as other modifications known in the art, both naturally occurring and non-naturally occurring (e.g., synthetic). Thus, synthetic peptides, dimers, multimers (e.g., tandem repeats, multiple antigenic peptide (MAP) forms, linearly-linked peptides), cyclized, branched molecules and the like, are included within the definition. The terms also include molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, peptides useful in this invention can have a maximum length suitable for the intended application. Preferably, the peptide is between about 3 and 100 residues in length. Generally, one skilled in art can easily select the maximum length in view of the teachings herein. Further, peptide reagents as described herein, for example synthetic peptides, may include additional molecules such as labels, linkers, or other chemical moieties (e.g., biotin, amyloid specific dyes such as Control Red or Thioflavin). Such moieties may further enhance interaction of the peptides with the prion proteins and/or further detection of prion proteins.

Peptide reagents also includes derivatives of the amino acid sequences of the invention having one or more substitution, addition and/or deletion, including one or more non-naturally occurring amino acid. Preferably, derivatives exhibit at least about 50% identity to any wild type or reference sequence, preferably at least about 70% identity, more preferably at least about 75%, 80%, 85%, 90%, 91%, 92%, 93%, 94%, 95%, 96%, 97%, 98%, 99% or 100% sequence identity to any wild type or reference sequence described herein. Sequence (or percent) identity can be determined as described below. Such derivatives can include postexpression modifications of the polypeptide, for example, glycosylation, acetylation, phosphorylation, and the like.

Peptide derivatives can also include modifications to the native sequence, such as deletions, additions and substitutions (generally conservative in nature), so long as the polypeptide maintains the desired activity. These modifications may be deliberate, as through site-directed mutagenesis, or may be accidental, such as through mutations of hosts that produce the proteins or errors due to PCR amplification. Furthermore, modifications may be made that have one or more of the following effects: reducing toxicity; increasing affinity and/or specificity for prion proteins; facilitating cell processing (e.g., secretion, antigen presentation, etc.); and facilitating presentation to B-cells and/or T-cells. Polypeptides described herein can be made recombinantly, synthetically, purified from natural sources, or in tissue culture.

A “fragment” as used herein refers to a peptide consisting of only a part of the intact full-length protein and structure as found in nature. For instance, a fragment can include a C-terminal deletion and/or an N-terminal deletion of a protein. Typically, the fragment retains one, some or all of the functions of the full-length polypeptide sequence from which it is derived. Typically, a fragment will comprise at least 5 consecutive amino acid residues of the native protein; preferably, at least about 8 consecutive amino acid residues; more preferably, at least about 10, 11, 12, 13, 14, 15, 16, 17, 18, 19, 20, 21, 22, 23, 24, 25, 26, 27, 28, 29, or 30 consecutive amino acid residues of the native protein.

The term “polynucleotide”, as known in the art, generally refers to a nucleic acid molecule. A “polynucleotide” can include both double- and single-stranded sequences and refers to, but is not limited to, prokaryotic sequences, eukaryotic mRNA, cDNA from viral, prokaryotic or eukaryotic mRNA, genomic RNA and DNA sequences from viral (e.g. RNA and DNA viruses and retroviruses), prokaryotic DNA or eukaryotic (e.g., mammalian) DNA, and especially synthetic DNA sequences. The term also captures sequences that include any of the known base analogs of DNA and RNA, and includes modifications such as deletions, additions and substitutions (generally conservative in nature), to the native sequence. These modifications may be deliberate, as through site-directed mutagenesis, or may be accidental, such as through mutations of hosts including prion-encoding polynucleotides. Modifications of polynucleotides may have any number of effects including, for example, facilitating expression of the polypeptide product in a host cell.

A polynucleotide can encode a biologically active (e.g., immunogenic or therapeutic) protein or polypeptide. Depending on the nature of the polypeptide encoded by the polynucleotide, a polynucleotide can include as little as 10 nucleotides, e.g., where the polynucleotide encodes an antigen or epitope. Typically, the polynucleotide encodes peptides of at least 18, 19, 20, 21, 22, 23, 24, 25, 30 or even more amino acids.

A “polynucleotide coding sequence” or a sequence that “encodes” a selected polypeptide, is a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Typical “control elements,” include, but are not limited to, transcription regulators, such as promoters, transcription enhancer elements, transcription termination signals, and polyadenylation sequences; and translation regulators, such as sequences for optimization of initiation of translation, e.g., Shine-Dalgarno (ribosome binding site) sequences, Kozak sequences (i.e., sequences for the optimization of translation, located, for example, 5′ to the coding sequence), leader sequences (heterologous or native), translation initiation codon (e.g., ATG), and translation termination sequences. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

A “recombinant” nucleic acid molecule as used herein to describe a nucleic acid molecule means a polynucleotide of genomic, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

“Antibody” as known in the art includes one or more biological moieties that, through chemical or physical means, can bind to or associate with an epitope of a polypeptide of interest. For example, the antibodies of the invention may interact preferentially with (e.g., specifically bind to) pathogenic prion conformations. The term “antibody” includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; Fᵥ molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The term “antibody” further includes antibodies obtained through non-conventional processes, such as phage display.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

If polyclonal antibodies are desired, a selected mammal (e.g., mouse, rabbit, goat, horse, etc.) is generally immunized with an immunogenic composition (e.g., a peptide reagent as described herein). Serum from the immunized animal is collected and treated according to known procedures. If serum containing polyclonal antibodies to the selected peptide reagent contains antibodies to other antigens, the polyclonal antibodies can be purified by immunoaffinity chromatography. Techniques for producing and processing polyclonal antisera are known in the art, see for example, Mayer and Walker, eds. (1987) IMMUNOCHEMICAL METHODS IN CELL AND MOLECULAR BIOLOGY (Academic Press, London).

One skilled in the art can also readily produce monoclonal antibodies directed against peptide reagents described herein. The general methodology for making monoclonal antibodies by hybridomas is well known. Immortal antibody-producing cell lines can be created by cell fusion, and also by other techniques such as direct transformation of B-lymphocytes with oncogenic DNA, or transfection with Epstein-Barr virus. See, e.g., M. Schreier et al. (1980) HYBRIDOMA TECHNIQUES; Hammerling et al. (1981), MONOCLONAL ANTIBODIES AND T-CELL HYBRIDOMAS; Kennett et al. (1980) MONOCLONAL ANTIBODIES; see also, U.S. Pat. Nos. 4,341,761; 4,399,121; 4,427,783; 4,444,887; 4,466,917; 4,472,500; 4,491,632; and 4,493,890.

As used herein, a “single domain antibody” (dAb) is an antibody that is comprised of an VH domain, which binds specifically with a designated antigen. A dAb does not contain a VL domain, but may contain other antigen binding domains known to exist to antibodies, for example, the kappa and lambda domains. Methods for preparing dabs are known in the art. See, for example, Ward et al, Nature 341: 544 (1989).

Antibodies can also be comprised of VH and VL domains, as well as other known antigen binding domains. Examples of these types of antibodies and methods for their preparation are known in the art (see, e.g., U.S. Pat. No. 4,816,467, which is incorporated herein by reference), and include the following. For example, “vertebrate antibodies” refers to antibodies that are tetramers or aggregates thereof, comprising light and heavy chains which are usually aggregated in a “Y” configuration and which may or may not have covalent linkages between the chains. In vertebrate antibodies, the amino acid sequences of the chains are homologous with those sequences found in antibodies produced in vertebrates, whether in situ or in vitro (for example, in hybridomas). Vertebrate antibodies include, for example, purified polyclonal antibodies and monoclonal antibodies, methods for the preparation of which are described infra.

“Hybrid antibodies” are antibodies where chains are separately homologous with reference to mammalian antibody chains and represent novel assemblies of them, so that two different antigens are precipitable by the tetramer or aggregate. In hybrid antibodies, one pair of heavy and light chains are homologous to those found in an antibody raised against a first antigen, while a second pair of chains are homologous to those found in an antibody raised against a second antibody. This results in the property of “divalence”, i.e., the ability to bind two antigens simultaneously. Such hybrids can also be formed using chimeric chains, as set forth below.

“Chimeric antibodies” refers to antibodies in which the heavy and/or light chains are fusion proteins. Typically, one portion of the amino acid sequences of the chain is homologous to corresponding sequences in an antibody derived from a particular species or a particular class, while the remaining segment of the chain is homologous to the sequences derived from another species and/or class. Usually, the variable region of both light and heavy chains mimics the variable regions or antibodies derived from one species of vertebrates, while the constant portions are homologous to the sequences in the antibodies derived from another species of vertebrates. However, the definition is not limited to this particular example. Also included is any antibody in which either or both of the heavy or light chains are composed of combinations of sequences mimicking the sequences in antibodies of different sources, whether these sources be from differing classes or different species of origin, and whether or not the fusion point is at the variable/constant boundary. Thus, it is possible to produce antibodies in which neither the constant nor the variable region mimic known antibody sequences. It then becomes possible, for example, to construct antibodies whose variable region has a higher specific affinity for a particular antigen, or whose constant region can elicit enhanced complement fixation, or to make other improvements in properties possessed by a particular constant region.

Another example is “altered antibodies”, which refers to antibodies in which the naturally occurring amino acid sequence in a vertebrate antibody has been varies. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics, e.g., changes in complement fixation, interaction with membranes, and other effector functions. Changes in the variable region can be made to alter antigen-binding characteristics. The antibody can also be engineered to aid the specific delivery of a molecule or substance to a specific cell or tissue site. The desired alterations can be made by known techniques in molecular biology, e.g., recombinant techniques, site-directed mutagenesis, etc.

Yet another example are “univalent antibodies”, which are aggregates comprised of a heavy-chain/light-chain dimer bound to the Fc (i.e., stem) region of a second heavy chain. This type of antibody escapes antigenic modulation. See, e.g., Glennie et al. Nature 295: 712 (1982). Included also within the definition of antibodies are “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)2), which are capable of selectively reacting with a designated antigen or antigen family. Fab antibodies can be divided into subsets analogous to those described above, i.e., “vertebrate Fab”, “hybrid Fab”, “chimeric Fab”, and “altered Fab”. Methods of producing Fab fragments of antibodies are known within the art and include, for example, proteolysis, and synthesis by recombinant techniques.

“Antigen-antibody complex” refers to the complex formed by an antibody that is specifically bound to an epitope on an antigen.

A peptide (or peptide reagent) is said to “interact” with another peptide or protein if it binds specifically, non-specifically or in some combination of specific and non-specific binding. A peptide (or peptide reagent) is said to “interact preferentially” with a pathogenic prion protein if it bind with greater affinity and/or greater specificity to the pathogenic form than to nonpathogenic isoforms. A peptide reagent that interacts preferentially with a pathogenic prion protein is also referred to herein as a pathogenic prion-specific peptide reagent. It is to be understood that a preferential interaction does not necessarily require interaction between specific amino acid residues and/or motifs of each peptide. For example, in certain embodiments, the peptide reagents described herein interact preferentially with pathogenic isoforms but, nonetheless, may be capable of binding nonpathogenic isoforms at a weak, yet detectable, level (e.g., 10% or less of the binding shown to the polypeptide of interest). Typically, weak binding, or background binding, is readily discernible from the preferentially interaction with the compound or polypeptide of interest, e.g., by use of appropriate controls. In general, peptides of the invention bind pathogenic prions in the presence of 10⁶-fold excess of nonpathogenic forms.

The term “affinity” refers to the strength of binding and can be expressed quantitatively as a dissociation constant (K_{d}). Preferably, a peptide (or peptide reagent) that interacts preferentially with a pathogenic isoform preferably interacts with the pathogenic isoform with at least 2 fold greater affinity, more preferably at least 10 fold greater affinity and even more preferably at least 100 fold greater affinity than it interacts with the nonpathogenic isoform. Binding affinity (i.e., K_{d}) can be determined using standard techniques.

Techniques for determining amino acid sequence “similarity” or “percent identity” are well known in the art. In general, “similarity” means the amino acid to amino acid comparison of two or more polypeptides at the appropriate place, where amino acids are identical or possess similar chemical and/or physical properties such as charge or hydrophobicity. A so-termed “percent identity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more amino acid or polynucleotide sequences can be compared by determining their “percent identity.” Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH™ package of programs copyrighted by the University of Edinburgh, developed by John F. Collins and Shane S. Sturrok, and available from numerous sources, for example on the internet. From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

An “immunogenic composition” as used herein refers to any composition (e.g., peptide, antibody and/or polynucleotides) where administration of the composition to a subject results in the development in the subject of a humoral and/or a cellular immune response. The immunogenic composition can be introduced directly into a recipient subject, such as by injection, inhalation, oral, intranasal or any other parenteral or mucosal (e.g., intra-rectally or intra-vaginally) route of administration.

By “epitope” is meant a site on an antigen to which specific B cells and/or T cells respond, rendering the molecule including such an epitope capable of eliciting an immunological reaction or capable of reacting with antibodies present in a biological sample. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” or “immune response” as used herein is the development in the subject of a humoral and/or a cellular immune response to a peptide as described herein when the polypeptide is present in a vaccine composition. These antibodies may also neutralize infectivity, and/or mediate antibody-complement or antibody dependent cell cytotoxicity to provide protection to an immunized host. Immunological reactivity may be determined in standard immunoassays, such as a competition assays, well known in the art.

“Gene transfer” or “gene delivery” refers to methods or systems for reliably inserting DNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from alphaviruses, pox viruses and vaccinia viruses. When used for immunization, such gene delivery expression vectors may be referred to as vaccines or vaccine vectors.

The term “sample” includes biological and non-biological samples. Biological samples are those obtained or derived from a living or once-living organism. Non-biological samples are not derived from living or once-living organisms. Biological samples include, but are not limited to, samples derived from an animal (living or dead) such as organs (e.g., brain, liver, kidney, etc), whole blood, blood fractions, plasma, cerebrospinal fluid (CSF), urine, tears, tissue, organs, biopsies. Examples of non-biological samples include pharmaceuticals, foods, cosmetics and the like.

The terms “label” and “detectable label” refer to a molecule capable of detection, including, but not limited to, radioactive isotopes, fluorescers, luminescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to fluorescein, rhodamine, dansyl, umbelliferone, Texas red, luminol, acridinium esters, NADPH, beta-galactosidase, horseradish peroxidase, glucose oxidase, alkaline phosphatase and urease. The label can also be an epitope tag (e.g., a His-His tag), an antibody or an amplifiable or otherwise detectable oligonucleotide.

### II. GENERAL OVERVIEW

Described herein are methods for detecting a pathogenic prion in a sample using a peptide reagent in which the peptide reagent is capable of distinguishing between pathogenic and nonpathogenic isoforms of prion proteins, for example by preferentially interacting with one form and not the other. Utilizing these peptide reagents, the present inventors have developed a sensitive method for detecting the presence of pathogenic prions in a sample. The peptide reagents are described herein and are also described in co-owned applications U.S. Ser. No. 10/917,646, filed Aug. 13, 2004; U.S. Ser. No. 11/056,950, filed Feb. 11, 2005; and PCT application No. PCT/US2004/026363, filed Aug. 13, 2004. Because the peptide reagents preferentially interact with the pathogenic form of the prion, they can be used to effectively separate and concentrate the pathogenic prions from samples containing both cellular (i.e., non-pathogenic) prion proteins and pathogenic prion proteins. Unlike previously described methods for detecting PrP^{sc} no digestion with proteinase K or other protease is necessary. The peptide reagents are typically provided on a solid support, preferably a magnetic bead, in order to readily accomplish separation of pathogenic prion proteins, which are bound to the peptide reagent, from other components of the sample, especially from non-pathogenic prion proteins. The bound pathogenic prions may optionally be washed to remove any trace of unbound materials. The bound pathogenic prions can then be dissociated from the peptide reagent by addition of chaotropic agents or preferably by changing the pH.

### III. A. PEPTIDE REAGENTS

The invention relies in part on the discovery by the present inventors that relatively small fragments of a prion protein can interact preferentially with the pathogenic form of the prion. These fragments need not be part of a larger protein structure or other type of scaffold molecule in order to exhibit this preferential interaction with the pathogenic prion isoform. While not wanting to be held to any particular theory, it appears that the peptide fragments spontaneously take on a conformation that allows binding to the pathogenic prion isoform but not to the nonpathogenic prion isoform, perhaps by mimicking a conformation that is present in the nonpathogenic isoform. This general principle, that certain fragments of a conformational disease protein interact preferentially with the pathogenic form of that conformational disease protein, here demonstrated for prions, can readily be applied to other conformational disease proteins to produce peptide reagents that interact preferentially with the pathogenic forms. It will be apparent to one of ordinary skill in the art that, while the fragments provide a starting point (in terms of size or sequence characteristics, for example), that many modifications can be made on the fragments to produce peptide reagents with more desirable attributes (e.g., higher affinity, greater stability, greater solubility, less protease sensitivity, greater specificity, easier to synthesize, etc.).

In general, the peptide reagents described herein are able to interact preferentially with pathogenic forms of prion proteins. Thus, these peptide reagents allow for ready detection of the presence of pathogenic prion proteins and, hence, diagnosis of prion-related diseases in virtually any sample, biological or non-biological, including living or dead brain, spinal cord, or other nervous system tissue as well as blood.

In addition, any suitable signal amplification system can be used to further facilitate detection, including but not limited to, the use of branched DNA for signal amplification (see, e.g., U.S. Pat. Nos. 5,681,697; 5,424,413; 5,451,503; 5,4547,025; and 6,235,483); applying target amplification techniques like PCR, rolling circle amplification, Third Wave's invader (Arruda et al. 2002 Expert. Rev. Mol. Diagn. 2:487; U.S. Pat. Nos. 6,090,606, 5,843,669, 5,985,557, 6,090,543, 5,846,717), NASBA, TMA etc. (U.S. Pat. No. 6,511,809; EP 0544212A1); and/or immuno-PCR techniques (see, e.g., U.S. Pat. No. 5,665,539; International Publications WO 98/23962; WO 00/75663; and WO 01/31056).

Described herein are peptide reagents that interact with pathogenic forms of a conformational disease protein. Conformational disease proteins are exemplified herein by prion proteins.

The following is a non-limiting list of diseases with associated proteins that assume two or more different conformations.

| | Conformational |
|---|---|
| Disease | Disease Protein(s) |
| Prion diseases | PrP^{Sc} |
| (e.g., Creutzfeld Jakob disease, scrapie, | |
| bovine spongiform encephalopathy) | |
| Alzheimer's Disease | APP, A* peptide, |
| | *1-antichymotrypsin, |
| | tan, non-A* component |
| ALS | SOD and neurofilament |
| Pick's disease | Pick body |
| Parkinson's disease | Lewy body |
| Diabetes Type 1 | Amylin |
| Multiple myeloma - plasma cell dyscrasias | IgGL-chain |
| Familial amyloidotic polyneuropathy | Transthyretin |
| Medullary carcinoma of thyroid | Procalcitonin |
| Chronic Renal failure | beta2-microglobulin |
| Congestive heart failure | atrial natriuretic factor |
| senile cardiac and systemic amyloidosis | Transthyretin |
| Chronic inflammation | Serum amyloid A |
| Atherosclerosis | ApoA1 |
| Familial amyloidosis | Gelsolin |

Further, the conformational disease proteins listed above each include a number of variants or mutations that result in different strains that are all encompassed by the present invention. Functional analysis of various regions and sequences of a mouse prion protein are given below. See, also, Priola (2001) Adv. Protein Chem. 57:1-27. Regions and residues corresponding to those set forth below for mouse (Mo), hamster (Ha), human (Hu), avian (A) and sheep (Sh) can readily be determined for other species following standard procedures and the teachings herein.

| Amino | |
|---|---|
| Acid(s) | Function |
| Mol-28 | Translocation domain (cleaved) |
| 22 | Putative cleavage site |
| 23-28 | Basic region potentially interacting with Protein X binding |
| | site as its deletion abrogates the effect of protein X |
| | associated mutations in the C-terminus of prion proteins. |
| 23-88 | Octarepeat region (1-9 insertions or 2 deletions potentiate |
| | disease); Copper coordination by the histidines in each of |
| | the repeats |
| 34-52 | Portion of Octarepeat shown for form a polyproline helix |
| | and also to form hydroxyproline |
| 86-91 | Cleavage sites of PrP^{Sc} when Proteinase K digests |
| Hu82-146 | 7Kda fragment found in diseased brains of GSS patients; |
| | synthetic peptide corresponding to this region forms ion |
| | channels |
| HuP102 | P102L mutation associated with GSS, does not appear to |
| | cause spontaneous conversion of the prion protein to |
| | protease resistant conformation; Proline conserved in all |
| | species examined. |
| HuP105 | P105L mutation associated with GSS, does not appear to |
| | cause spontaneous conversion of the prion protein to |
| | protease resistant conformation; Proline conserved in all |
| | species examined. |
| Hu102-105 | PXXP motif; possible polyproline type II helix |
| Mo_106 | Associated with disease resistance |
| Hu106-126 | Mutant forms of synthetic peptides suggested to form |
| | copper modulating ion channels; G114 and G119 shown to |
| | decrease fibrillogenic behavior of this peptide as peptide is |
| | more amyloidogenic when they are mutated to A. |
| Mo_111 | Associated with disease resistance |
| Sh104-113 | Peptide co-crystallized with D13 Fab |
| Ha109-112 | Loop specifically recognized by D13 peptide as shown in |
| | crystal structure (M109 and M112 are inserted into binding |
| | pockets within the Fab). |
| Hu113-120 | Palindromic sequence; totally conserved |
| A117V | Pathogenic mutation in palindrome; increases |
| | amyloidogenic properties of peptides containing that |
| | region |
| Ha129-131 | Beta sheet 1 in PrP^{C} |
| Hu129/ | Polymorphism associated with susceptibility/resistance to |
| Go132 | prion disease |
| Ha136 | Alanine polymorphism associated with increase in coated |
| | pits in sheep |
| Mo138/ | Polymorphism associated with susceptibility/resistance to |
| Go142 | prion disease |
| Mo141-176 | Area deleted (along with 23-88) in mouse miniprion |
| | PrP106 has no effect; suggests non-essential function for |
| | this region |
| Ha144-154 | Helix A |
| Ha155 | Polymorphism associated with susceptibility/resistance to |
| | prion disease |
| Ha160-163 | Sheet 2 |
| MoV165 | Species barrier; when human transgenic mice mutate these |
| | residues back to mouse sequence, much faster incubation |
| | times are obtained |
| MoQ167 | Species barrier; when human transgenic mice mutate these |
| | residues back to mouse sequence, much faster incubation |
| | times are obtained |
| MoQ168 | Putative Protein X binding site; when mutated it protects |
| | against prion disease |
| Sh171 | Polymorphism associated with susceptibility/resistance to |
| | prion disease |
| MoQ172 | Putative Protein X binding site; when mutated it protects |
| | against prion disease |
| 176 | Disulfide-linked cysteine. |
| Ha173-194 | Helix B |
| 178 | Disease-associated mutation |
| 180 | Disease-associated mutation; glycosylation site |
| 196 | Glycosylation site |
| 198 | Disease-associated mutation |
| Hu200-228 | Helix C |
| HuE200 | Mutation to K associated with familial CJD in Lybian Jews |
| | (M129 polymorphism in combo also increases chances of |
| | disease) |
| 208 | Disease-associated mutation |
| 210 | Disease-associated mutation |
| MoT215 | Putative Protein X binding site; when mutated it protects |
| | against prion disease |
| 217 | Disease-associated mutation |
| MoQ219 | Putative Protein X binding site; when mutated it protects |
| | against prion disease |
| 232 | Disease-associated mutation |
| 232 | GPI anchor |
| ~233 | Putative GPI anchor cleavage site |
| 233-254 | Portion removed from mature protein |

It should also be noted that prion proteins (and other conformational disease proteins) have two different 3-dimensional conformations with the same amino acid sequence. One conformation is associated with disease characteristics and is generally insoluble whereas the other conformation is not associated with disease characteristics and is soluble. See, e.g., Wille, et al., “Structural Studies of the Scrapie Prion Protein by Electron Crystallography”, Proc. Natl. Acad. Sci. USA, 99 (6): 3563-3568 (2002). Although exemplified with respect to prion proteins, the present invention is not limited to the diseases, proteins and strains listed.

Thus, in certain aspects, the peptide reagents described herein comprise an amino acid sequence derived from a naturally occurring protein, for example a conformational disease protein (e.g., prion protein) or a protein that contains motifs or sequences that exhibit homology to prion proteins. In particular, the peptide reagents of the invention are typically derived from a naturally-occurring prion protein. The peptide reagents are preferably derived from the amino acid sequences from certain regions of the prion proteins. These preferred regions are exemplified with respect to the mouse prion sequence (SEQ ID NO:2), in regions from amino acid residue 23-43 and 85-156, and subregions thereof. The invention is not limited to peptide reagents derived from the mouse sequences but include peptide reagents derived in similar fashion as described herein, from prion sequences of any species, including human, bovine, sheep, deer, elk, hamster. When derived from prion proteins, the peptide reagents described herein may include a polyproline type II helix motif. This motif typically contains the general sequence PxxP (e.g., residues 102-105 of SEQ ID NO:1), although other sequences, in particular alanine tetrapeptides, have been suggested to form polyproline type II helices as well (see, e.g., Nguyen et al. Chem. Biol. 2000 7:463; Nguyen et al. Science 1998 282:2088; Schweitzer-Stenner et al. J. Am. Chem. Soc. 2004 126:2768). In the PxxP sequence, “x” can be any amino acid and “P” is proline in the naturally occurring sequence but may be replaced by a proline substitute in the peptide reagents of the invention. Such proline substitutes include N-substituted glycines commonly referred to as peptoids. Thus, in the peptide reagents of the invention that include a polyproline type II helix based on the PxxP sequence, “P” represents a proline or an N-substituted glycine residues and “x” represents any amino acid or amino acid analog. Particularly preferred N-substituted glycines are described herein.

Further, the polynucleotide and amino acid sequence for prion proteins produced by many different species are known, including human, mouse, sheep and cattle. Variants to these sequences also exist within each species. Thus, the peptide reagents used in the invention can comprise fragments or derivatives of the amino acid sequences of any species or variant. For example, in certain embodiments, the peptide reagents described herein are derived from any of the sequences set forth in FIG. 2 (SEQ ID NOs:3-11). The sequences of the peptide reagents that are specifically disclosed herein are generally based on the mouse prion sequence, however, one skilled in the art can readily substitute corresponding sequences from other species when appropriate. For example, if human diagnostics or therapeutics are desired, replacement of the mouse sequences with those of the corresponding human sequences can be easily done. In a particular example, in peptide reagents derived from the region from about residue 85 to about residue 112 (e.g., SEQ ID NO:35, 36, 37, 40), the leucine at position corresponding to residue 109 may be replaced with a methionine, the valine at position corresponding to residue 112 may be replaced with methionine, and the asparagine at position corresponding to 97 may be replaced with serine. Likewise, if a bovine diagnostic is desired, the appropriate substitutions may be made in the disclosed peptide sequences to reflect the bovine prion sequence. Thus, continuing with the above example for peptide reagents derived from the region from about residue 85 to about residue 112, the leucine at position corresponding to residue 109 may be replaced with a methionine and the asparagine at position corresponding to 97 may be replaced with glycine. Derivatives of prion proteins, including amino acid replacements, deletions, additions and other mutations to these sequences can also be used. Preferably, any amino acid replacements, additions, and deletions as compared to a prion protein sequence do not affect the ability of the peptide reagent to interact with pathogenic form.

It should be understood that no matter what source is used for the peptide reagents described herein, these peptide reagents will not necessarily exhibit sequence identity to known prion proteins. Thus, the peptide reagents described herein can include one or more amino acid replacements, additions, and deletions relative to the naturally occurring prion protein or the sequences disclosed herein, so long as they retain the ability to interact preferentially with pathogenic forms of conformational disease proteins. In certain embodiments, conservative amino acid replacements are preferred. Conservative amino acid replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity.

It will also be apparent that any combination of the natural amino acids and non-natural amino acid analogs can be used to make the peptide reagents described herein. Commonly encountered amino acid analogs that are not gene-encoded include, but are not limited to, ornithine (Orn); aminoisobutyric acid (Aib); benzothiophenylalanine (BtPhe); albizziin (Abz); t-butylglycine (Tle); phenylglycine (PhG); cyclohexylalanine (Cha); norleucine (Nle); 2-naphthylalanine (2-Nal); 1-naphthylalanine (1-Nal); 2-thienylalanine (2-Thi); 1,2,3,4-tetrahydroisoquinoline-3-carboxylic acid (Tic); N-methylisoleucine (N-MeIle); homoarginine (Har); Nα-methylarginine (N-MeArg); phosphotyrosine (pTyr or pY); pipecolinic acid (Pip); 4-chlorophenylalanine (4-CIPhe); 4-fluorophenylalanine (4-FPhe); 1-aminocyclopropanecarboxylic acid (1-NCPC); and sarcosine (Sar). Any of the amino acids used in the peptide reagents of the present invention may be either the D- or, more typically, L-isomer.

Other non-naturally occurring analogs of amino acids that may be used to form the peptide reagents described herein include peptoids and/or peptidomimetic compounds such as the sulfonic and boronic acid analogs of amino acids that are biologically functional equivalents are also useful in the compounds of the present invention and include compounds having one or more amide linkages optionally replaced by an isostere. In the context of the present invention, for example, —CONH— may be replaced by —CH₂NH—, —NHCO—, —SO₂NH—, —CH₂O—, —CH₂CH₂—, —CH₂S—, —CH₂SO—, —CH—CH— (cis or trans), —COCH₂—, —CH(OH)CH₂— and 1,5-disubstituted tetrazole such that the radicals linked by these isosteres would be held in similar orientations to radicals linked by —CONH—. One or more residues in the peptide reagents described herein may comprise peptoids.

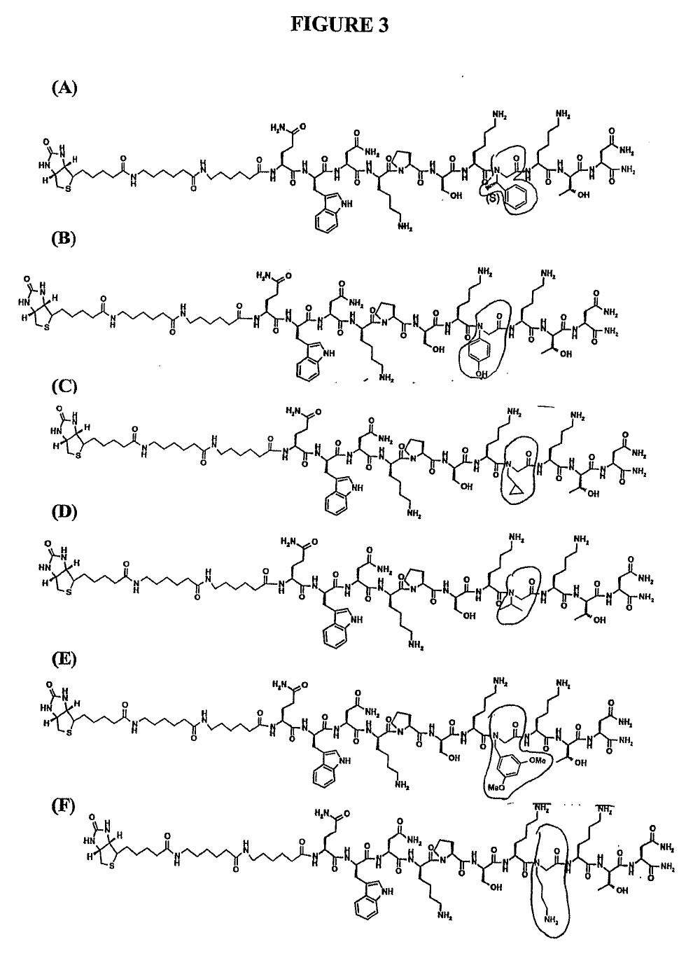

Thus, the peptide reagents also may comprise one or more N-substituted glycine residues (peptides having one or more N-substituted glycine residues may be referred to as “peptoids”). For example, in certain embodiments, one or more proline residues of any of the peptide reagents described herein are replaced with N-substituted glycine residues. Particular N-substituted glycines that are suitable in this regard include, but are not limited to, N—(S)-(1-phenylethyl)glycine; N-(4-hydroxyphenyl)glycine; N-(cyclopropylmethyl)glycine; N-(isopropyl)glycine; N-(3,5-dimethoxybenzyl)glycine; and N-amino butylglycine. (e.g., FIG. 3). Other N-substituted glycines may also be suitable to replace one or more amino acid residues in the peptide reagents sequences described herein. For a general review of these and other amino acid analogs and peptidomimetics see, Nguyen et al. (2000) Chem. Biol. 7(7):463-473; Spatola, A. F., in Chemistry and Biochemistry of Amino Acids, Peptides and Proteins, B. Weinstein, eds., Marcel Dekker, New York, p. 267 (1983). See also, Spatola, A. F., Peptide Backbone Modifications (general review), Vega Data, Vol. 1, Issue 3, (March 1983); Morley, Trends Pharm Sci (general review), pp. 463-468 (1980); Hudson, D. et al., Int Pept Prot Res, 14:177-185 (1979) (—CH₂NH—, CH₂CH₂—); Spatola et al., Life Sci, 38:1243-1249 (1986) (—CH₂—S); Hann J. Chem. Soc. Perkin Trans. I, 307-314 (1982) (—CH—CH—, cis and trans); Almquist et al., J Med Chem, 23:1392-1398 (1980) (—COCH₂—); Jennings-White et al., Tetrahedron Lett, 23:2533 (1982) (—COCH₂—); Szelke et al., European Appln. EP 45665 CA: 97:39405 (1982) (—CH(OH)CH₂—); Holladay et al., Tetrahedron Lett, 24:4401-4404 (1983) (—C(OH)CH₂—); and Hruby, Life Sci, 31:189-199 (1982) (—CH₂—S—); each of which is incorporated herein by reference. The C-terminal carboxylic acid can be replaced by a boronic acid —B(OH)₂ or boronic ester —B(OR)₂ or other such boronic acid derivative as disclosed in U.S. Pat. No. 5,288,707, incorporated herein by reference.

The peptide reagents described herein may comprise monomers, multimers, cyclized molecules, branched molecules, linkers and the like. Multimers (i.e., dimers, trimers and the like) of any of the sequences described herein or biologically functional equivalents thereof are also contemplated. The multimer can be a homomultimer, i.e., composed of identical monomers, e.g., each monomer is the same peptide sequence. Alternatively, the multimer can be a heteromultimer, by which is meant that not all the monomers making up the multimer are identical.

Multimers can be formed by the direct attachment of the monomers to each other or to substrate, including, for example, multiple antigenic peptides (MAPS) (e.g., symmetric MAPS), peptides attached to polymer scaffolds, e.g., a PEG scaffold and/or peptides linked in tandem with or without spacer units.

Alternatively, linking groups can be added to the monomeric sequences to join the monomers together and form a multimer. Non-limiting examples of multimers using linking groups include tandem repeats using glycine linkers; MAPS attached via a linker to a substrate and/or linearly linked peptides attached via linkers to a scaffold. Linking groups may involve using bifunctional spacer units (either homobifunctional or heterobifunctional) as are known to one of skill in the art. By way of example and not limitation, many methods for incorporating such spacer units in linking peptides together using reagents such as succinimidyl-4-(p-maleimidomethyl)cyclohexane-1-carboxylate (SMCC), succinimidyl-4-(p-maleimidophenyl)butyrate and the like are described in the Pierce Immunotechnology Handbook (Pierce Chemical Co., Rockville, Ill.) and are also available from Sigma Chemical Co. (St. Louis, Mo.) and Aldrich Chemical Co. (Milwaukee, Wis.) and described in “Comprehensive Organic Transformations”, VCK-Verlagsgesellschaft, Weinheim/Germany (1989). One example of a linking group which may be used to link the monomeric sequences together is —Y₁—F—Y₂ where Y₁ and Y₂ are identical or different and are alkylene groups of 0-20, preferably 0-8, more preferably 0-3 carbon atoms, and F is one or more functional groups such as —O—, —S—, —S—S—, —C(O)—O—, —NR—, —C(O)—NR—, —NR—C(O)—O—, —NR—C(O)—NR—, —NR—C(S)—NR—, —NR—C(S)—O—. Y₁ and Y₂ may be optionally substituted with hydroxy, alkoxy, hydroxyalkyl, alkoxyalkyl, amino, carboxyl, carboxyalkyl and the like. It will be understood that any appropriate atom of the monomer can be attached to the linking group.

Further, the peptide reagents of the invention may be linear, branched or cyclized. Monomer units can be cyclized or may be linked together to provide the multimers in a linear or branched fashion, in the form of a ring (for example, a macrocycle), in the form of a star (dendrimers) or in the form of a ball (e.g., fullerenes). Skilled artisans will readily recognize a multitude of polymers that can be formed from the monomeric sequences disclosed herein. In certain embodiments, the multimer is a cyclic dimer. Using the same terminology as above, the dimer can be a homodimer or a heterodimer.

Cyclic forms, whether monomer or multimer, can be made by any of the linkages described above, such as but not limited to, for example: (1) cyclizing the N-terminal amine with the C-terminal carboxylic acid either via direct amide bond formation between the nitrogen and the C-terminal carbonyl, or via the intermediacy of spacer group such as for example by condensation with an epsilon-amino carboxylic acid; (2) cyclizing via the formation of a bond between the side chains of two residues, e.g., by forming a amide bond between an aspartate or glutamate side chain and a lysine side chain, or by disulfide bond formation between two cysteine side chains or between a penicillamine and cysteine side chain or between two penicillamine side chains; (3) cyclizing via formation of an amide bond between a side chain (e.g., aspartate or lysine) and either the N-terminal amine or the C-terminal carboxyl respectively; and/or (4) linking two side chains via the intermediacy of a short carbon spacer group.

Preferably, the peptide reagents described herein are not pathogenic and/or infectious.

The peptide reagents of the invention can be anywhere from 3 to about 100 residues long (or any value therebetween) or even longer, preferably from about 4 to 75 residues (or any value therebetween), preferably from about 5 to about 63 residues (or any value therebetween), and even more preferably from about 8 to about 30 residues (or any value therebetween), and most preferably the peptide reagent will be 10, 11, 12, 13, 14, 15, 16, 17, 18, 19, 20, 21, 22, 23, 24, 25, 26, 27, 28, 29 or 30 residues.

Non-limiting examples of peptide reagents useful in the compositions and methods described herein are derived from sequences shown in Table 1 and in Table 4. Peptide reagents in the tables are represented by conventional one letter amino acid codes and are depicted with their N-terminus at the left and C-terminus at the right. Amino acids in square brackets indicate alternative residues that can be used at that position in different peptide reagents. Round brackets indicate the residue(s) may be present or absent from the peptide reagent. Any proline residue may be substituted with N-substituted glycine residues to form peptoids. Any of the sequences in the tables may optionally include Gly linkers (Gₙ where n=1, 2, 3, or 4) at the N- and/or C-terminal.

TABLE 1

| | SEQ |
|---|---|
| | ID |
| Peptide sequence | NO |
| KKRPK | 12 |
| MANLGCWMLVLFVATWSDLGLC | 13 |
| (GGG)QWNKPSKPKTN | 14 |
| QWNKPSKPKTNMKHV | 15 |
| NQNN[N/T]FVHDCVNIT[I/V]K[Q/E]HTVTTTTKGEN | 16 |
| TTKGENFTETD | 17 |
| GENFTETD | 18 |
| GENFTETD[V/I]K[M/I]MERVVEQMC[I/V]TQY[E/Q]ESQAYY | 19 |
| [Q/D] (G)(R)R[G/S][S/A]S | |
| NQNN[N/T]FVHDCVNIT[I/V]K[Q/E]HTVTTTTKGENFTETD | 20 |
| [V/I]K[M/I]MERVVEQMC[I/V]TQY[E/Q]ESQAYY[Q/D](G) | |
| (R)R[G/S][S/A]S | |
| [A/V/T/M][V/I]LFSSPPVILLISFLIFL[I/M]VG | 21 |
| G[N/S]D[W/Y]EDRYYRENM[H/Y]RYPNQVYYRP[M/V]D[Q/E/ | 22 |
| R]Y[S/N]NQN[N/T] FVH | |
| N[N/T]FVHDCVNIT[I/V]K[Q/E]HTVTTTTK | 23 |
| VYYR | 24 |
| RYPNQVYYRP[M/V]D[Q/E/R] | 25 |
| KKRPKPGG(G)WNTGGSRYPGQGSPGGNRYPPQGG | 26 |
| WNTGGSRYPGQGSPGGNRYPPQGG(G) | 27 |
| WNTGGSRYPGQGSPGGNRYPPQGG(G)[G/T]WGQPHGG | 28 |
| GGWGQGGGTHSQWNKPSKPKTN | 29 |
| GGTHSQWNKPSKPKTN | 30 |
| WNTGGSRYPGQGSPGGNRYPPQGG(G)[G/T]WGQPHGGGWGQ | 31 |
| PHGGGWGQPHGG | |
| GQPHGGGW | 32 |
| RPIIHFGSDYEDRYYRENMHR | 33 |
| RPMIHFGNDWEDRYYRENMYR | 34 |
| (GGGG)C(GG)GGWGQGGGTHNQWNKPSKPKTNLKHV(GGGG) | 35 |
| C | |
| (GGGG)GGWGQGGGTHNQWNKPSKPKTNLKHV | 36 |
| GGWGQGGGTHNQWNKPSKPKTNLKHV(GGGG) | 37 |
| [M/L]KH[M/V] | 38 |
| KPKTN[M/L]KH[M/V] | 39 |
| C(GG)GGWGQGGGTHNQWNKPSKPKTNLKHV(GGGG)C | 40 |
| SRPIIHFGSDYEDRYYRENMHRYPN | 41 |
| PMIHFGNDWEDRYYRENMYRPVD | 42 |
| AGAAAAGAVVGGLGGYMLGSAM | 43 |
| RPMIHFGNDWEDRYYRENMYR(GGG) | 44 |
| GGGRPMIHFGNDWEDRYYRENMYRGG | 45 |
| (GG)C(GGG)RPMIHFGNDWEDRYYRENMYR(GGG)C | 46 |
| AGAAAAGAVVGGLGG | 47 |
| GGLGG | 48 |
| LGS | 49 |
| QWNKPSKPKTN(GGG) | 50 |
| QWNKPSKPKTN(GGG)WNKPSKPKTN | 51 |
| QWNKPSKPKTNLKHV(GGG) | 52 |
| GGWGQGGGTHNQWNKPSKPKTN | 53 |
| GGTHNQWNKPSKPKTN | 54 |
| (GGG)AGAAAAGAVVGGLGGYMLGSAM | 55 |
| (GGG)AGAAAAGAVVGGLGG | 56 |
| (KKK)AGAAAAGAVVGGLGGYMLGSAM | 57 |
| YMLGSAM[S/N]R | 58 |
| [S/N]RP[M/I/L][I/L]H | 59 |
| YMLGSAM[S/N]RP[M/I/L][I/L]H | 60 |
| YMLGSAM[S/N]RP[M/I/L][I/L]HFG[N/S]D | 61 |
| [W/Y]EDRYYRENM[H/Y]RYPNQVYYRP[M/V]D[Q/E/R]Y | 62 |
| [W/Y]EDRYYRENM[H/Y]RYPNQVYYRP[M/V]D[Q/E/R]Y[S/ | 63 |
| N]NQN[N/T] | |
| D[Q/E/R]Y[S/N]NQN[N/T] | 64 |
| (KKK)AGAAAAGAVVGGLGG | 65 |
| (GGG)KKRPKPGGWNTGGSRYPGQGS | 66 |
| (GGG)KKRPKPGGWNTGG | 67 |
| (GGG)KKRPKPGG | 68 |
| PHGGGWGQHGGSWGQPHGGSWGQ | 69 |
| PHGGGWGQPHGGSWGQ | 70 |
| PHGGGWGQ | 71 |
| (GGG)KKRPKPGGGKKRPKPGG | 72 |
| (GGG)GPKRKGPK | 73 |
| (GGG)WNTGGSRYPGQGS | 74 |
| (GGG)WNKPSKPKT | 75 |
| (GGG)RPMIHFGNDWEDRYYRENMYR(GG)C | 76 |
| QWNKPSKPKTNLKHV(GGG) | 77 |
| (GGG)AGAAAAGAVVGGLGGYMLGSAM | 78 |
| (GGG)NKPSKPK | 79 |
| (GGG)KPSKPK | 80 |
| (GGG)KKRPKPGGGQWNKPSKPKTN | 81 |
| KKKAGAAAAGAVVGGLGGYMLGSAMDDD | 82 |
| DDDAGAAAAGAYVGGLGGYMLGSAM | 83 |
| KKKAGAAAAGAVVGGLGGYMLGSAMKKK | 84 |
| (GGG)KKKKKKKK | 85 |
| DDDAGAAAAGAVVGGLGGYMLGSAMDDD | 86 |
| (GGG)NNKQSPWPTKK | 87 |
| DKDKGGVGALAGAAVAAGGDKDK | 88 |
| (GGG)QANKPSKPKTN | 89 |
| (GGG)QWNKASKPKTN | 90 |
| (GGG)QWNKPSKAKTN | 91 |
| (GGG)QWNAPSKPKTN | 92 |
| (GGG)QWNKPSAPKTN | 93 |
| (GGG)QWNKPSKPATN | 94 |
| (GGG)QWNKASKAKTN | 95 |
| (GGG)KKRAKPGG | 96 |
| (GGG)KKRPKAGG | 97 |
| (GGG)KKRAKAGG | 98 |
| (GGG)QWNKASKPKTN | 99 |
| (GGG)QWAKPSKPKTN | 100 |
| (GGG)QWNKPAKPKTN | 101 |
| (GGG)QWNKPSKPKAN | 102 |
| (GGG)QWNKPSKPKTA | 103 |
| (GGG)AKRPKPGG | 104 |
| (GGG)KARPKPGG | 105 |
| (GGG)KKAPKPGG | 106 |
| (GGG)KKRPAPGG | 107 |
| (GGG)KKAPKAGG | 108 |
| (GGG)KKRPKPGGGWNTGG | 127 |
| QWNKPSKPKTNGGGQWNKPSKPKTNGGGQWNKPSKPKTN | 128 |
| ((QWNKPSKPKTN))2K | 133 |
| 4-branchMAPS-GGGKKRPKPQGWNTGGG | 134 |
| 8-branchMAPS-GGGKKRPKPGGWNTGGG | 135 |
| KKKAGAAAAGAVVGGLGG-CONH2 | 136 |
| DLGLCKKRPKPGGXWNTGG | 137 |
| DLGLCKKRPKPGGXWNTG | 138 |
| DLGLCKKRPKPGGXWNT | 139 |
| DLGLCKKRPKPGGXWN | 140 |
| DLGLCKKRPKPGGXW | 141 |
| DLGLCKKRPKPGGX | 142 |
| LGLCKKRPKPGGXWNTG | 143 |
| LGLCKKRPKPGGXWNT | 144 |
| LGLCKKRPKPGGXWN | 145 |
| LGLCKKRPKPGGXW | 146 |
| LGLCKKRPKPGGX | 147 |
| GLCKKRPKPGGXWNTGG | 148 |
| GLCKKRPKPGGXWNTG | 149 |
| GLCKKRPKPGGXWNT | 150 |
| GLCKKRPKPGGXWN | 151 |
| GLCKKRPKPGGXW | 152 |
| GLCKKRPKPGGX | 153 |
| LCKKRPKPGGXWNTGG | 154 |
| LCKKRPKPGGXWNTG | 155 |
| LCKKRPKPGGXWNT | 156 |
| LCKKRPKPGGXWN | 157 |
| LCKKRPKPGGXW | 158 |
| LGKKRPKPGGX | 159 |
| CKKRPKPGGXWNTGG | 160 |
| CKKRPKPGGXWNTG | 161 |
| CKKRPKPGGXWNT | 162 |
| CKKRPKPGGXWN | 163 |
| CKKRPKPGGXW | 164 |
| CKKRPKPGGX | 165 |
| KKRPKPGGXWNTGG | 166 |
| KKRPKPGGXWNTG | 167 |
| KKRPKPGGXWNT | 168 |
| KKRPKPGGXWN | 169 |
| KKRPKPGGXW | 170 |
| KKRPKPGGX | 171 |
| DVGLCKKRPKPGGXWNTGG | 172 |
| DVGLCKKRPKPGGXWNTG | 173 |
| DVGLCKKRPKPGGXWNT | 174 |
| DVGLCKKRPKPGGXWN | 175 |
| DVGLCKKRPKPGGXW | 176 |
| DVGLCKKRPKPGGX | 177 |
| VGLCKKRPKPGGXWNTG | 178 |
| VGLCKKRPKPGGXWNT | 179 |
| VGLCKKRPKPGGXWN | 180 |
| VGLCKKRPKPGGXW | 181 |
| VGLCKKRPKPGGX | 182 |
| THSQWNKPSKPKTNMKHM | 183 |
| THSQWNKPSKPKTNMKH | 184 |
| THSQWNKPSKPKTNMK | 185 |
| THSQWNKPSKPKTNM | 186 |
| THSQWNKPSKPKTN | 187 |
| HSQWNKPSKPKTNMKHM | 188 |
| HSQWNKPSKPKTNMKH | 189 |
| HSQWNKPSKPKTNMK | 190 |
| HSQWNKPSKPKTNM | 191 |
| HSQWNKPSKPKTN | 192 |
| SQWNKPSKPKTNMKHM | 193 |
| SQWNKPSKPKTNMKH | 194 |
| SQWNKPSKPKTNMK | 195 |
| SQWNKPSKPKTNM | 196 |
| SQWNKPSKPKTN | 197 |
| QWNKPSKPKTNMKHM | 198 |
| QWNKPSKPKTNMKH | 199 |
| QWNKPSKPKTNMK | 200 |
| QWNKPSKPKTNM | 201 |
| THSQWNKPSKPKTNMKHV | 202 |
| HSQWNKPSKPKTNMKHV | 203 |
| SQWNKPSKPKTNMKHV | 204 |
| QWNKPSKPKTNMKHV | 205 |
| THGQWNKPSKPKTNMKHM | 206 |
| THGQWNKPSKPKTNMKH | 207 |
| THGQWNKPSKPKTNMK | 208 |
| THGQWNKPSKPKTNM | 209 |
| THGQWNKPSKPKTN | 210 |
| HGQWNKPSKPKTNMKHM | 211 |
| HGQWNKPSKPKTNMKH | 212 |
| HGQWNKPSKPKTNMK | 213 |
| HGQWNKPSKPKTNM | 214 |
| HGQWNKPSKPKTN | 215 |
| GQWNKPSKPKTNMKHM | 216 |
| GQWNKPSKPKTNMKH | 217 |
| GQWNKPSKPKTNMK | 218 |
| GQWNKPSKPKTNM | 219 |
| GQWNKPSKPKTN | 220 |
| THGQWNKPSKPKTNMKHV | 221 |
| HGQWNKPSKPKTNMKHV | 222 |
| GQWNKPSKPKTNMKHV | 223 |
| THNQWNKPSKPKTNMKHM | 224 |
| THNQWNKPSKPKTNMKH | 225 |
| THNQWNKPSKPKTNMK | 226 |
| THNQWNKPSKPKTNM | 227 |
| THNQWNKPSKPKTN | 228 |
| HNQWNKPSKPKTNMKHM | 229 |
| HNQWNKPSKPKTNMKH | 230 |
| HNQWNKPSKPKTNMK | 231 |
| HNQWNKPSKPKTNM | 232 |
| HNQWNKPSKPKTN | 233 |
| NQWNKPSKPKTNMKHM | 234 |
| NQWNKPSKPKTNMKH | 235 |
| NQWNKPSKPKTNMK | 236 |
| NQWNKPSKPKTNM | 237 |
| NQWNKPSKPKTN | 238 |
| THNQWNKPSKPKTNMKHV | 239 |
| HNQWNKPSKPKTNMKHV | 240 |
| NQWNKPSKPKTNMKHV | 241 |
| PHGGGWGQPHGGGWGQPHGGGWGQ | 242 |
| GGWGQGGGTHSQWNKPSKPKTNMKHM | 243 |
| QWNKPSKPKTNMKHMGGGQWNKPSKPKTNMKHM | 244 |
| GGWGQGGGTH[N/S]QWNKPSKPKTN[L/M]KH[V/M](GGGG) | 245 |
| PHGGGWGQHG[G/S]SWGQPHGG[G/S]WGQ | 246 |
| QWNKPSKPKTN[L/M]KH[V/M](GGG) | 247 |
| 4-branchMAPS-(GGG)QWNKPSKPKTN(GGG) | 259 |
| 8-branchMAPS-(GGG)KKRPKPGGWNT(GGG) | 260 |

In one aspect, the peptide reagent used in the method of the invention includes each of the peptides disclosed herein and derivatives (as described herein) thereof. The invention thus includes a peptide reagent derived from a peptide of any of the sequences shown in SEQ ID NO: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, or 260 and analogs (e.g., substitution of one or more proline with a N-substituted glycine) and derivatives thereof.

The method of the invention preferably utilizes a peptide reagent derived from a peptide of SEQ ID NO:12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 72, 74, 76, 77, 78, 81, 82, 84, 89, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 249, 250, 251, 252, 253, 254, 255, 256, 257, 258, 259, or 260 and analogs (e.g., substitution of one or more proline with a N-substituted glycine) and derivatives thereof.

In certain preferred embodiments, the peptide reagents used in the methods specifically bind to pathogenic prions, for example peptide reagents derived from peptides of SEQ ID NOs: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 56, 57, 65, 82, 84, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 249, 250, 251, 252, 253, 254, 255, 256, 257, 258, 259, or 260, and analogs (e.g., substitution of one or more proline with a N-substituted glycine) and derivatives thereof.

As described above, the peptide reagents described herein may include one or more substitutions, additions, and/or mutations. For example, one or more residues may be replaced in the peptide reagents with other residues, for example alanine residues or with an amino acid analog or N-substituted glycine residue in order to make a peptoid (see, e.g., Nguyen et al. (2000) Chem Biol. 7(7):463-473). Furthermore, the peptide reagents described herein may also include additional peptide or non-peptide components. Non-limiting examples of additional peptide components include spacer residues, for example two or more glycine (natural or derivatized) residues or aminohexanoic acid linkers on one or both ends or residues that may aid in solubilizing the peptide reagents, for example acidic residues such as aspartic acid (Asp or D) as depicted for example in SEQ ID NOs:83, 86. In certain embodiments, for example, the peptide reagents are synthesized as multiple antigenic peptides (MAPs). Typically, multiple copies of the peptide reagents (e.g., 2-10 copies) are synthesized directly onto a MAP carrier such as a branched lysine or other MAP carrier core. See, e.g., Wu et al. (2001) J Am Chem. Soc. 2001 123(28):6778-84; Spetzler et al. (1995) Int J Pept Protein Res. 45(1):78-85 and SEQ ID NOs:134 and 135.

Non-limiting examples of non-peptide components (e.g., chemical moieties) that may be included in the peptide reagents described herein include, one or more detectable labels, tags (e.g., biotin, His-Tags, oligonucleotides), dyes, members of a binding pair, and the like, at either terminus or internal to the peptide reagent. The non-peptide components may also be attached (e.g., via covalent attachment of one or more labels), directly or through a spacer (e.g., an amide group), to position(s) on the compound that are predicted by quantitative structure-activity data and/or molecular modeling to be non-interfering. Peptide reagents as described herein may also include prion-specific chemical moieties such as amyloid-specific dyes (e.g., Congo Red, Thioflavin, etc.). Derivatization (e.g., labeling, cyclizing, attachment of chemical moieties, etc.) of compounds should not substantially interfere with (and may even enhance) the binding properties, biological function and/or pharmacological activity of the peptide reagent.

The peptide reagents will typically have at least about 50% sequence identity to prion protein fragments or to the peptide sequences set forth herein. Preferably, the peptide reagents will have at least 70% sequence identity: more preferably at least 75, 80, 85, 90, 91, 92, 93, 94, 95, 96, 97, 98, 99, or 100% sequence identity to prion protein fragments or to the peptide sequences set forth herein.

The peptide reagents as described herein interact preferentially with the pathogenic forms and, accordingly, are useful in a wide range of isolation, purification, detection, diagnostic and therapeutic applications. For example, in embodiments in which the peptide reagent interacts preferentially with pathogenic forms, the peptide reagents themselves can be used to detect pathogenic forms in a sample, such as a blood, nervous system tissue (brain, spinal cord, CSF, etc) or other tissue or organ sample. The peptide reagents are also useful to diagnose the presence of disease associated with the pathogenic forms, to isolate the pathogenic forms and to decontaminate samples by removing the pathogenic forms.

The interaction of the peptide reagents with prion proteins can be tested using any known binding assay, for example immunoassays such as ELISAs, Western blots and the like (see, Examples).

One convenient method of testing the specificity of the peptide reagents of the present invention is to select a sample containing both pathogenic and non-pathogenic prions. Typical such samples include brain or spinal cord tissue from diseased animals. Peptide reagents as described herein that bind specifically to pathogenic forms are attached to a solid support (by methods well-known in the art and as further described below) and used to separate (“pull down”) pathogenic prion from the other sample components and obtain a quantitative value directly related to the number of peptide-prion binding interactions on the solid support. Variations and other assays known in the art can also be used to demonstrate the specificity of the peptide reagents of the invention. See, e.g., Examples.

Although not required in the method of the invention using the peptide reagents described herein, other prion assays may utilize the fact that prions having a pathogenic conformation are generally resistant to certain proteases, such as proteinase K. The same proteases are able to degrade prions in a non-pathogenic conformation. Therefore, when using a protease, the sample can be separated into two equal volumes. Protease can be added to the second sample and the same test performed. Because the protease in the second sample will degrade any non-pathogenic prions, any peptide-prion binding interactions in the second sample can be attributed to pathogenic prions.

Thus, non-limiting examples of methods of evaluating binding specificity and/or affinity of the peptide reagents described herein include standard Western and Far-Western Blotting procedures; labeled peptides; ELISA-like assays; and/or cell based assays. Western blots, for example, typically employ a tagged primary antibody that detects denatured prion protein from an SDS-PAGE gel, on samples obtained from a “pull-down” assay (as described herein), which has been electroblotted onto nitrocellulose or PVDF. Antibodies that recognize denatured prion protein have been described (described, inter alia, in Peretz et al. 1997 J. Mol. Biol. 273: 614; Peretz et al. 2001 Nature 412:739; Williamson et al. 1998 J. Virol. 72:9413; U.S. Pat. No. 6,765,088; U.S. Pat. No. 6,537,548) and some are commercially available. Other prion-binding molecules have been described e.g., motif-grafted hybrid polypeptides (see, WO03/085086), certain cationic or anionic polymers (see, WO03/073106), certain peptides that are “propagation catalysts” (see, WO02/0974444) and plasminogen. The primary antibody is then detected (and/or amplified) with a probe for the tag (e.g., streptavidin-conjugated alkaline phosphatase, horseradish peroxidase, ECL reagent, and/or amplifiable oligonucleotides). Binding can also be evaluated using detection reagents such as a peptide with an affinity tag (e.g., biotin) that is labeled and amplified with a probe for the affinity tag (e.g., streptavidin-conjugated alkaline phosphatase, horseradish peroxidase, ECL reagent, or amplifiable oligonucleotides). In addition, microtitre plate procedures similar to sandwich ELISA may be used, for example, a prion-specific peptide reagent as described herein is used to immobilize prion protein(s) on a solid support (e.g., well of a microtiter plate, bead, etc.) and an additional detection reagent which could include, but is not limited to, another prion-specific peptide reagent with an affinity and/or detection label such as a conjugated alkaline phosphatase, horseradish peroxidase, ECL reagent, or amplifiable oligonucleotides. See, Examples. Cell based assays can also be employed, for example, where the prion protein is detected directly on individual cells (e.g., using a fluorescently labeled prion-specific peptide reagent that enables fluorescence based cell sorting, counting, or detection of the specifically labeled cells).

### III.B. Peptide Reagent Production

The peptide reagents of the present invention can be produced in any number of ways, all of which are well known in the art.

In one embodiment, in which the peptide reagent is, in whole or in part, a genetically encoded peptide, the peptide can be generated using recombinant techniques, well known in the art. One of skill in the art could readily determining nucleotide sequences that encode the desired peptide using standard methodology and the teachings herein. Once isolated, the recombinant peptide, optionally, can be modified to include non-genetically encoded components (e.g., detectable labels, binding pair members, etc.) as described herein and as well-known in the art, to produce the peptide reagents.

Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding the peptide can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed peptide reagents that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci. USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the peptide reagents described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (E. L. V. Harris and S. Angal, Eds., 1990).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinuin agglutinin (AUA). The choice of a suitable affinity resin is within the skill in the art. After affinity purification, the polypeptides can be further purified using conventional techniques well known in the art, such as by any of the techniques described above.

Peptide reagents can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Synthesis of peptoid containing polymers can be carried out according to, e.g., U.S. Pat. Nos. 5,877,278; 6,033,631; Simon et al. (1992) Proc. Natl. Acad. Sci. USA 89:9367.

The peptide reagent of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### IV. ASSAYS

The present inventors have developed a sensitive assay for detecting pathogenic prions in a sample. The assay combines the power of the peptide reagents to discriminate between the pathogenic and the non-pathogenic form of the prion proteins with an improved ELISA technique. Because the peptide reagents interact preferentially with the pathogenic prion proteins, these reagents are used to separate and concentrate any pathogenic prion present in the sample. Unlike methods that utilize digestion with proteinase K to discriminate between the pathogenic and non-pathogenic isoforms, which typically results in some N-terminal digestion even of the pathogenic isoform, use of the peptide reagents in the method of the invention results in the separation of full-length pathogenic prion proteins. Thus, anti-prion antibodies that recognize epitopes at the N-terminal end of the prion protein can be used for detection, as well as anti-prion antibodies that recognize epitopes from other regions of the prion protein.

Once the pathogenic prion protein is separated from the non-pathogenic isoform (which is present in most samples) using the peptide reagents, the pathogenic prion protein can be dissociated from the peptide reagent and detected in a number of ELISA formats, described herein. The pathogenic prion is typically denatured in the process of dissociation from the peptide reagent. Use of a denatured prion protein in the ELISA is preferable as many anti-prion antibodies that bind to the denatured PrP are known and commercially available. The dissociation and denaturation of the pathogenic prion can be accomplished using high concentrations of chaotropic agents, e.g., 3M to 6M of a guanidium salt such as guanidinium thiocyanate or guanidinium HCl. The chaotropic agent must be removed or diluted before the ELISA is carried out because it will interfere with the binding of the anti-prion antibodies used in the ELISA. This results in additional washing steps or generation of large sample volumes, both of which are undesirable for rapid, high-throughput assays.

The present inventors have discovered that a preferable alternative to the use of a chaotropic agent for dissociation/denaturation of the pathogenic prion protein from the peptide reagent is the use of high or low pH. The pathogenic prion protein is readily dissociated from the peptide reagent and denatured by adding components that increase the pH to above 12 (e.g., NaOH) or to below 2 (e.g., H₃PO₄). Moreover, the pH can be easily readjusted to neutral by addition of small volumes of suitable acid or base, thus allowing the use directly in the ELISA without any additional washes and without increasing the sample volumes significantly.

The invention thus provides a method for detecting the presence of a pathogenic prion in a sample comprising: contacting the sample suspected of containing a pathogenic prion with a peptide reagent that interacts preferentially with the pathogenic form of the prion protein under conditions that allow the binding of the peptide reagent to the pathogenic prion protein, if present, to form a first complex; removing unbound sample material; dissociating the pathogenic prion from the peptide reagent; and detecting the presence the dissociated pathogenic prion using a prion-binding reagent. A “prion-binding reagent” is a reagent that binds to a prion protein in any conformation, typically the prion-binding reagent will bind to a denatured form of the prion protein. Such reagents have been described and include, for example, anti-prion antibodies (described, inter alia, in Peretz et al. 1997 J. Mol. Biol. 273: 614; Peretz et al. 2001 Nature 412:739; Williamson et al. 1998 J. Virol. 72:9413; U.S. Pat. No. 6,765,088; U.S. Pat. No. 6,537,548), motif-grafted hybrid polypeptides (see, WO03/085086), certain cationic or anionic polymers (see, WO03/073106), certain peptides that are “propagation catalysts” (see, WO02/0974444) and plasminogen. It will be apparent that if the particular prion-binding reagent used binds to a denatured form of the prion that the “captured” pathogenic prion protein should be denatured prior to detection with the prion-binding reagent. Preferably, the prion-binding reagent is an anti-prion antibody.

In certain embodiments, anti-PrP antibodies are used to detect prion proteins. Antibodies, modified antibodies and other reagents, that bind to prions, particularly to PrP^{C} or to the denatured PrP, have been described and some of these are available commercially (see, e.g., anti-prion antibodies described in Peretz et al. 1997 J. Mol. Biol. 273: 614; Peretz et al. 2001 Nature 412:739; Williamson et al. 1998 J. Virol. 72:9413; U.S. Pat. No. 6,765,088. Some of these and others are available commercially from, inter alia, InPro Biotechnology, South San Francisco, Calif., Cayman Chemicals, Ann Arbor Mich.; Prionics AG, Zurich; also see, WO 03/085086 for description of modified antibodies). Suitable antibodies for use in the method include without limitation 3F4, D18, D13, 6H4, MAB5242, 7D9, BDI115, SAF32, SAF53, SAF83, SAF84, 19B10, 7VC, 12F10, PRI308, 34C9, Fab HuM-P, Fab HuM-R1, and Fab HuM-R72.

Preferably the dissociated pathogenic prion protein is denatured. The term “denature” or “denatured” has the conventional meaning as applied to protein structure and means that the protein loses its native secondary and tertiary structure. With respect to the pathogenic prion protein, a “denatured” pathogenic prion protein no longer retains the native pathogenic conformation and thus the protein is no longer “pathogenic”. The denatured pathogenic prion protein has a conformation similar or identical to the denatured non-pathogenic prion protein. However, for purposes of clarity herein, the term “denatured pathogenic prion protein” will be used to refer to the pathogenic prion protein that is captured by the peptide reagent as the pathogenic isoform and subsequently denatured.

In preferred embodiments, the peptide reagent is provided on a solid support. The peptide reagent can be provided on a solid support prior to contacting the sample or the peptide reagent can be adapted for binding to the solid support after contacting the sample and binding to any pathogenic prion therein (e.g., by using a biotinylated peptide reagent and a solid support comprising an avidin or streptavidin).

The invention thus additionally provides a method for detecting the presence of a pathogenic prion in a sample comprising:

(a) providing a first solid support comprising a peptide reagent;

(b) contacting the first solid support with a sample under conditions that allow pathogenic prion proteins, when present in the sample, to bind to the peptide reagent to form a first complex;

(c) removing unbound sample material;

(d) dissociating the pathogenic prion proteins from the first complex; and

(e) detecting the dissociated pathogenic prions using a prion-binding reagent.

The peptide reagent is preferably derived from a peptide having a sequence selected from the group consisting of SEQ ID NO:12-260.

Methods of making a solid support comprising a peptide reagent are conventional in the art and are described elsewhere herein and include well-known methods of attaching proteins and peptides to various solid surfaces. The sample is contacted with the solid support comprising the peptide reagent under conditions that allow the binding of any pathogenic prion proteins in the sample to bind to the peptide reagent, forming a first complex. Such binding conditions are readily determined by one of ordinary skill in the art and are further described herein. Typically, the method is carried out in the wells of a microtiter plate or in small volume plastic tubes, but any convenient container will be suitable. The sample is generally a liquid sample or suspension and may be added to the reaction container before or after the peptide reagent. Once the first complex is established, unbound sample material (that is, any components of the sample that have not bound to the peptide reagent, including any unbound pathogenic prion protein) can be removed by separating the solid support from the reaction solution (containing the unbound sample materials) for example, by centrifugation, precipitation, filtration, magnetic force, etc. The solid support with the first complex may optionally be subjected to one or more washing steps to remove any residual sample materials before carrying out the next steps of the method.

Following the removal of unbound sample materials and any optional washes, the bound pathogenic prion proteins are dissociated from the first complex. This dissociation can be accomplished in a number of ways. In one embodiment, a chaotropic agent, preferably a guanidinium compound, e.g., guanidinium thiocyanate or guanidinium hydrochloride, is added to a concentration of between 3M and 6M. Addition of the chaotropic agent causes the pathogenic prion protein to dissociate from the peptide reagent and also causes the pathogenic prion protein to denature.

In another embodiment, the dissociation is accomplished by either raising the pH to 12 or above (“high pH”) or lowering the pH to 2 or below (“low pH”). Exposure of the first complex to either high or low pH results in the dissociation of the pathogenic prion protein from the peptide reagent and causes the pathogenic prion protein to denature. In this embodiment, exposure of the first complex to high pH is preferred. A pH of between 12.0 and 13.0 is generally sufficient; preferably, a pH of between 12.5 and 13.0 is used; more preferably, a pH of 12.7 to 12.9; most preferably a pH of 12.9. Alternatively, exposure of the first complex to a low pH can be used to dissociate and denature the pathogenic prion protein from the peptide reagent. For this alternative, a pH of between 1.0 and 2.0 is sufficient. Exposure of the first complex to either a high pH or a low pH is carried out for only a short time, for example 60 minutes, preferably for no more than 15 minutes, more preferably for no more than 10 minutes. Longer exposures than this can result in significant deterioration of the structure of the pathogenic prion protein such that epitopes recognized by anti-prion antibodies used in the detection steps are destroyed. After exposure for sufficient time to dissociate the pathogenic prion protein, the pH can be readily readjusted to neutral (that is, pH of between about 7.0 and 7.5) by addition of either an acidic reagent (if high pH dissociation conditions are used) or a basic reagent (if low pH dissociation conditions are used). One of ordinary skill in the art can readily determine appropriate protocols, and examples are described herein.

In general, to effect a high pH dissociation condition, addition of NaOH to a concentration of about 0.05 N to about 0.2 N is sufficient. Preferably, NaOH is added to a concentration of between 0.05 N to 0.15 N; more preferably, 0.1 N NaOH is used. Once the dissociation of the pathogenic prion from the peptide reagent is accomplished, the pH can be readjusted to neutral (that is, between about 7.0 and 7.5) by addition of suitable amounts of an acidic solution, e.g., phosphoric acid, sodium phosphate monobasic.

In general, to effect a low pH dissociation condition, addition of H₃PO₄ to a concentration of about 0.2 M to about 0.7 M is sufficient. Preferably, H₃PO₄ is added to a concentration of between 0.3 M and 0.6 M; more preferably, 0.5 M H₃PO₄ is used. Once the dissociation of the pathogenic prion from the peptide reagent is accomplished, the pH can be readjusted to neutral (that is, between about 7.0 and 7.5) by addition of suitable amounts of a basic solution, e.g., NaOH or KOH.

The dissociated pathogenic prion protein is then separated from the solid support comprising the peptide reagent. By “separated” is intended that the dissociated prion and the solid support (with bound peptide reagent) are not present together in the same container. This separation can be accomplished in similar fashion to the removal of the unbound sample materials described above.

The dissociated pathogenic prion protein can be detected using prion-binding reagents. A number of such prion-binding agents are known and described herein elsewhere. Preferred prion-binding reagents for detection of the dissociated pathogenic prion protein are anti-prion antibodies. A number of anti-prion antibodies have been described and many are commercially available, for example, Fab D18 (Peretz et al. (2001) Nature 412:739-743), 3F4 (available from Sigma Chemical St Louis Mo.; also, See, U.S. Pat. No. 4,806,627), SAF-32 (Cayman Chemical, Ann Arbor Mich.), 6H4 (Prionic AG, Switzerland; also, See U.S. Pat. No. 6,765,088). The dissociated pathogenic prion proteins can be detected in an ELISA type assay, either as a direct ELISA or a antibody Sandwich ELISA type assay, which are described more fully below. Although the term “ELISA” is used to describe the detection with anti-prion antibodies, the assay is not limited to ones in which the antibodies are “enzyme-linked.” The detection antibodies can be labeled with any of the detectable labels described herein and well-known in the immunoassay art.

In one embodiment of the method, the dissociated pathogenic prion protein is passively coated onto the surface of a second solid support. Methods for such passive coating are well known and typically are carried out in 100 mM NaHCO₃ at pH 8 for several hours at about 37° C. or overnight at 4° C. Other coating buffers are well-known (e.g, 50 mM carbonate pH 9.6, 10 mM Tris pH 8, or 10 mM PBS pH 7.2) The second solid support can be any of the solid supports described herein or well-known in the art; preferably the second solid support is a microtiter plate, e.g., a 96-well polystyrene plate. Where the dissociation has been carried out using a high concentration of chaotropic agent, the concentration of the chaotropic agent will be reduced by dilution by about 2-fold prior to coating on the second solid support. Where the dissociation has been carried out using a high or low pH, followed by neutralization, the dissociated pathogenic prion protein can be used for coating without any further dilution.

Once the dissociated pathogenic prion protein is coated onto the second solid support, the support can be washed to remove any components that are not adhered to the solid support. Anti-prion antibodies are added under conditions that allow for binding of the antibodies to the prion protein coated on the second solid support. If the dissociated pathogenic prion protein has been denatured prior to coating on the second solid support, the antibodies used will be ones that bind to the denatured form of the prion protein. Such antibodies include ones that are well known (such as those described above) as well as antibodies that are generated by well known methods, e.g., by using rPrP, PrPC or fragments thereof, to elicit an immune reaction in mice, rabbits, rats, etc. (See, U.S. Pat. Nos. 4,806,627; 6,165,784; 6,528,269; 6,379,905; 6,261,790; 6,765,088; 5,846,533; EP891552B1 and EP 909388B1). Anti-prion antibodies that recognize epitopes at the N-terminal end of the prion protein are particularly preferred, for example, antibodies that recognize epitopes within the region of residues 23-90.

Thus, the invention in one embodiment provides a method for detecting the presence of a pathogenic prion in a sample comprising:
(a) providing a first solid support comprising a peptide reagent;
(b) contacting the first solid support with a sample under conditions that allow pathogenic prion proteins, when present in the sample, to bind to the peptide reagent to form a first complex;
(c) removing unbound sample material;
(d) dissociating the pathogenic prion proteins from the first complex;
(e) separating the dissociated pathogenic prion proteins from the first solid support;
(f) contacting the dissociated pathogenic prion proteins with a second solid support under conditions that allow the dissociated prion protein to adhere to the second solid support; and
(g) detecting the adhered pathogenic prions on the second solid support using a prion-binding reagent. Preferred peptide reagents are those that are derived from a peptide having a sequence selected from the group consisting of SEQ ID NO:12-260.

In this embodiment, the first solid support is preferably a magnetic bead; the second solid support is preferably a microtiter plate; the prion-binding reagent is preferably an anti-prion antibody, particularly 3F4, 6H4, SAF32. The prion-binding reagent is detectably labeled.

In another embodiment of the method, the dissociated pathogenic prion proteins are detected using an antibody sandwich type ELISA. In this embodiment, the dissociated prion protein is “recaptured” on a second solid support comprising a first anti-prion antibody. The second solid support with the recaptured prion protein, is optionally washed to remove any unbound materials, and then contacted with a second anti-prion antibody under conditions that allow the second anti-prion antibody to bind to the recaptured prion protein. The first and second anti-prion antibodies will typically be different antibodies and will preferably recognize different epitopes on the prion protein. For example, the first anti-prion antibody will recognize an epitope at the N-terminal end of the prion protein and the second anti-prion antibody will recognize an epitope at other than the N-terminal, or vice versa. The first antibody can be, for example, SAF32 which recognizes an epitope in the octarepeat region (residues 23-90) and the second antibody can be 3F4, which recognizes an epitope at residues 109-112; alternatively, the first antibody can be 3F4 and the second antibody can be SAF32. Other combinations of first and second antibody can be readily selected. In this embodiment, the second anti-prion antibody, but not the first anti-prion antibody, will be detectably labeled. When the dissociation of the pathogenic prion protein from the peptide reagent is carried out using a chaotropic agent, the chaotropic agent must be removed or diluted by at least 15-fold prior to carrying out the detection assay. When the dissociation is effected using a high or low pH and neutralization, the dissociated prion can be used without further dilution. When the dissociated pathogenic prion protein is denatured prior to carrying out the detection, the first and second antibodies will both bind to the denatured prion protein. The invention thus provides a method for detecting the presence of a pathogenic prion in a sample comprising:
(a) providing a first solid support comprising a peptide reagent;
(b) contacting the first solid support with a sample under conditions that allow pathogenic prion proteins, when present in the sample, to bind to the peptide reagent to form a first complex;
(c) removing unbound sample material;
(d) dissociating the pathogenic prion proteins from the first complex, whereby the pathogenic prion protein is denatured;
(e) separating the dissociated denatured pathogenic prion proteins from the first solid support;
(f) contacting the dissociated denatured pathogenic prion proteins with a second solid support, wherein the second solid support comprises a first anti-prion antibody, under conditions that allow the dissociated prion protein to bind to the first anti-prion antibody; and
(g) detecting the bound prion proteins on the second solid support with a second anti-prion antibody. Preferred peptide reagents are those that are derived from a peptide having a sequence selected from the group consisting of SEQ ID NO:12-260.

In this embodiment, the first solid support is preferably a magnetic bead; the second solid support is preferably a microtiter plate or a magnetic bead; the first and second anti-prion antibodies are preferably different antibodies; the first and second antibodies bind to denatured prion protein; preferably, at least one of the first or second anti-prion antibodies recognizes an epitope at the N-terminal region of the prion protein.

For use in the method of the invention, the sample can be anything known to, or suspected of, containing a pathogenic prion protein. The sample can be a biological sample (that is, a sample prepared from a living or once-living organism) or a non-biological sample. Suitable biological samples include, but are not limited to, organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. In general, the sample will be a liquid sample or a suspension. Preferred biological samples include whole blood, blood fractions, blood components, plasma, platelets, and serum.

The sample is contacted with one or more peptide reagents of the invention under conditions that allow the binding of the peptide reagent(s) to the pathogenic prion protein if it is present in the sample. It is well within the competence of one of ordinary skill in the art to determine the particular conditions based on the disclosure herein. Typically, the sample and the peptide reagent(s) are incubated together in a suitable buffer at about neutral pH (e.g., a TBS buffer at pH 7.5) at a suitable temperature (e.g., about 4° C.), for a suitable time period (e.g., about 1 hour to overnight) to allow the binding to occur.

The above-described capture and detection steps can be carried out in solution or can be carried out in or on a solid support, or some combination of solution and solid phase. Suitable solid phase assay formats are described herein. In general, for solid phase formats, the capture reagent (which can be one or more of the peptide reagents of the invention, or one or more prion-binding reagents) is attached, or adapted for attachment, to a solid support. The capture reagent can be adapted for attachment to a solid support by any means known in the art, for example, the capture reagent and the solid support can each comprise one member of a binding pair, such that when the capture reagent is contacted with the solid support the capture reagent is attached to the solid support through the binding of the members of the binding pair. For example, the capture reagent can comprise biotin and the support can comprise avidin or streptavidin. In addition to biotin-avidin and biotin-streptavidin, other suitable binding pairs for this embodiment include, for example, antigen-antibody, hapten-antibody, mimetope-antibody, receptor-hormone, receptor-ligand, agonist-antagonist, lectin-carbohydrate, Protein A-antibody Fc. Such binding pairs are well known (see, e.g., U.S. Pat. Nos. 6,551,843 and 6,586,193) and one of ordinary skill in the art would be competent to select suitable binding pairs and adapt them for use with the present invention. When the capture reagent is adapted for attachment to the support as described above, the sample can be contacted with the capture reagent before or after the capture reagent is attached to the support. Alternatively, the peptide reagents and anti-prion antibodies can be covalently attached to the solid support using conjugation chemistries that are well known in the art. Thiol containing peptide reagents are directly attached to solid supports, e.g., magnetic beads, using standard methods known in the art (See, e.g., Chrisey, L. A., Lee, G. U. and O'Ferrall, C. E. (1996). Covalent attachment of synthetic DNA to self-assembled monolayer films. Nucleic Acids Research 24(15), 3031-3039; Kitagawa, T., Shimozono, T., Aikawa, T., Yoshida, T. and Nishimura, H. (1980). Preparation and characterization of hetero-bifunctional cross-linking reagents for protein modifications. Chem. Pharm. Bull. 29(4), 1130-1135). Carboxylated magnetic beads are first coupled to a heterobifunctional cross-linker that contains a maleimide functionality (BMPH from Pierce Biotechnology Inc.) using carbodiimide chemistry. The thiolated peptide or peptoid is then covalently coupled to the maleimide functionality of the BMPH coated beads.

The peptide reagent used in the method of the invention is as described herein and in co-owned applications U.S. Ser. No. 10/917,646, filed Aug. 13, 2004; U.S. Ser. No. 11/056,950, filed Feb. 11, 2005; and PCT application No. PCT/US2004/026363, filed Aug. 13, 2004. The peptide reagent can be derived from peptide fragments of a prion protein. Preferably the peptide reagent is derived from a peptide having a sequence of SEQ ID NO:12-260, that is, the peptide reagent is derived from a peptide having a sequence of SEQ ID NO:12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, or 260. More preferably, the peptide reagent used in the method is derived from a peptide having a sequence of one of SEQ ID NO: 66, 67, 68, 72, 81, 96, 97, 98, 107, 108, 119, 120, 121, 122, 123, 124, 125, 126, 127, 129, 130, 131, 132, 134 or 135; or from peptides having SEQ ID NO:14, 35, 36, 37, 40, 50, 51, 77, 89, 100, 101, 109, 110, 111, 112, 113, 114, 115, 116, 117, 118, 128 or 133; or from peptides having SEQ ID NO:56, 57, 65, 82, 84 or 136; most preferably, the peptide reagent is derived from a peptide having a sequence of SEQ ID NO:68, 50 or 14. The peptide reagent can be biotinylated. The peptide reagent can be attached to a solid support. In some embodiments, the peptide reagent can be detectably labeled.

Generally, peptide reagents as described herein are used to bind to prion proteins in a sample (e.g., as a capture reagent) and/or to detect the presence of prion proteins (e.g., as a detection reagent). The capture reagent and detection reagent may be separate molecules or, alternatively one molecule may serve both capture and detection functions. In certain embodiments, the capture and/or detection reagents are peptide reagents described herein that interact preferentially with pathogenic prions (i.e., are pathogenic-prion specific). In other embodiments, the capture reagent is specific for pathogenic prions and the detection reagent binds to both pathogenic and nonpathogenic forms, for example antibodies that bind to prion proteins. Such prion-binding reagents have been described above herein. Alternatively, in other embodiments, the capture reagent is not specific for pathogenic prions and the detection reagent is specific for pathogenic prions.

Any suitable means of detection can then be used to identify binding between a peptide reagent as described herein and a prion protein. For example, assays as described herein may involve the use of labeled peptide reagents or antibodies. Detectable labels suitable for use in the invention include any molecule capable of detection, including, but not limited to, radioactive isotopes, fluorescers, chemiluminescers, chromophores, fluorescent semiconductor nanocrystals, enzymes, enzyme substrates, enzyme cofactors, enzyme inhibitors, chromophores, dyes, metal ions, metal sols, ligands (e.g., biotin, strepavidin or haptens) and the like. Additional labels include, but are not limited to, those that use fluorescence, including those substances or portions thereof that are capable of exhibiting fluorescence in the detectable range. Particular examples of labels that may be used in the invention include, but are not limited to, alkaline phosphatase (AP), horse radish peroxidase (HRP), fluorescein, FITC, rhodamine, dansyl, umbelliferone, dimethyl acridinium ester (DMAE), Texas red, luminol, NADPH and β-galactosidase. In addition, the detectable label may include an oligonucleotide tag, which tag can be detected by any known method of nucleic acid detection including PCR, TMA, b-DNA, NASBA, etc.

One or more of the steps of the assays described herein may be conducted in solution (e.g., a liquid medium) or on a solid support. A solid support, for purposes of the invention, can be any material that is an insoluble matrix and can have a rigid or semi-rigid surface to which a molecule of interest (e.g., peptide reagents of the invention, prion proteins, antibodies, etc) can be linked or attached. Exemplary solid supports include, but are not limited to, substrates such as nitrocellulose, polyvinylchloride, polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride, diazotized paper; activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer. Particularly preferred solid supports are polystyrene microtiter plates and/or polystyrene magnetic particles, e.g., Dynabeads M-270 (Dynal Biotech).

Peptide reagents as described herein can be readily coupled to the solid support using standard techniques. Immobilization to the support may be enhanced by first coupling the peptide reagent to a protein (e.g., when the protein has better solid phase-binding properties). Suitable coupling proteins include, but are not limited to, macromolecules such as serum albumins including bovine serum albumin (BSA), keyhole limpet hemocyanin, immunoglobulin molecules, thyroglobuline, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to proteins, are well known to those of ordinary skill in the art. See, e.g., Brinkley, M. A., (1992) Bioconjugate Chem., 3:2-13; Hashida et al. (1984) J. Appl. Biochem., 6:56-63; and Anjaneyulu and Staros (1987) International J. of Peptide and Protein Res. 30:117-124.

If desired, the molecules to be added to the solid support can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose and the like.

The peptide reagents can be attached to the solid support through the interaction of a binding pair of molecules. Such binding pairs are well known and examples are described elsewhere herein. One member of the binding pair is coupled by techniques described above to the solid support and the other member of the binding pair is attached to the peptide reagent (before, during, or after synthesis). The peptide reagent thus modified can be contacted with the sample and interaction with the pathogenic prion, if present, can occur in solution, after which the solid support can be contacted with the peptide reagent (or peptide-prion complex). Preferred binding pairs for this embodiment include biotin and avidin, and biotin and streptavidin.

Suitable controls can also be used in the assays of the invention. For instance, a negative control of PrP^{C} can be used in the assays. A positive control of PrP^{Sc} (or PrPres) could also be used in the assays. Surrogate controls, described below, also find use in the invention.

The above-described assay reagents, including the peptide reagents described herein, can be provided in kits, with suitable instructions and other necessary reagents, in order to conduct detection assays as described above. Where the peptide reagent is conjugated onto a solid support, the kit may additionally or alternatively comprise such peptide reagents conjugated onto one or more solid supports. The kit may additionally contain one or more anti-prion antibody. Such anti-prion antibody may be detectably labeled or may be provided on a solid support. The kit may further contain suitable positive and negative controls, as described above. The kit can also contain, depending on the particular detection assay used, suitable labels and other packaged reagents and materials (i.e., wash buffers, incubation buffers).

### V. SURROGATE CONTROLS

Described herein are surrogate control molecules useful in prion detection assays. Compositions comprising the surrogate controls and methods of using these surrogates are also provided. Although artificial controls for immunoassays have been previously described (see, e.g., U.S. Pat. Nos. 5,846,738; 5,491,218; 6,015,662; 6,281,004 and International Patent Publication WO 99/33965), these molecules are not applicable to prion assays and cannot serve as controls.

In certain aspects, the surrogate control binds to a peptide reagent that interacts preferentially with a pathogenic prion protein. Thus, in these aspects, the invention (surrogate controls and methods of using same) relies in part on the discovery, as set forth in applications U.S. Ser. No. 10/917,646, filed Aug. 13, 2004; U.S. Ser. No. 11/056,950, filed Feb. 11, 2005; and PCT application No. PCT/US2004/026363, filed Aug. 13, 2004, that relatively small fragments of a prion protein can interact preferentially with the pathogenic form of the prion. These fragments need not be part of a larger protein structure or other type of scaffold molecule in order to exhibit this preferential interaction with the pathogenic prion isoform. While not wanting to be held to any particular theory, it appears that the peptide fragments spontaneously take on a conformation that allows binding to the pathogenic prion isoform but not to the nonpathogenic prion isoform, perhaps by mimicking a conformation that is present in the nonpathogenic isoform. This general principle, that certain fragments of a conformational disease protein interact preferentially with the pathogenic form of that conformational disease protein, here demonstrated for prions, can readily be applied to other conformational disease proteins to produce peptide reagents that interact preferentially with the pathogenic forms. It will be apparent to one of ordinary skill in the art that, while the fragments provide a starting point (in terms of size or sequence characteristics, for example), that many modifications can be made on the fragments to produce peptide reagents with more desirable attributes (e.g., higher affinity, greater stability, greater solubility, less protease sensitivity, greater specificity, easier to synthesize, etc.).

Thus, the surrogate controls described herein bind to prion-binding reagents, including peptide reagents as described in International Application number PCT/US2004/026363, filed Aug. 13, 2004 as well as antibodies (or fragments thereof) to these peptide reagents and/or other prion antibodies. Accordingly, these surrogate controls provide a simple and efficient non-infectious positive and/or quality controls for prion assays and can be used to confirm a diagnosis of prion-related diseases in virtually any sample, biological or non-biological, including living or dead brain, spinal cord, or other nervous system tissue as well as blood.

In addition, any suitable signal amplification system can be used to further facilitate detection of the surrogate controls in the assays, including but not limited to, the use of multiple recognition sites, branched DNA for signal amplification (see, e.g., U.S. Pat. Nos. 5,681,697; 5,424,413; 5,451,503; 5,4547,025; and 6,235,483); applying target amplification techniques like PCR, rolling circle amplification, Third Wave's invader (Arruda et al. 2002 Expert. Rev. Mol. Diagn. 2:487; U.S. Pat. Nos. 6,090,606, 5,843,669, 5,985,557, 6,090,543, 5,846,717), NASBA, TMA etc. (U.S. Pat. No. 6,511,809; EP 0544212A1); and/or immuno-PCR techniques (see, e.g., U.S. Pat. No. 5,665,539; International Publications WO 98/23962; WO 00/75663; and WO 01/31056).

Described herein are non-infectious molecules that act as surrogate controls for prion detection assays, particularly for assays that detect pathogenic prions in a sample. The surrogate controls described herein are useful as positive controls to confirm the accuracy of prion detection/isolation methods and/or as quality controls to insure that assay reagents and methods conform to the standards under which the assay is qualified.

In general, the assays for which the described surrogate controls are most useful utilize a prion-binding reagent, which can be, inter alia, a peptide reagent that interacts preferentially with a pathogenic prion protein, to “capture” the prion to be detected. By “capture” is intended to mean immobilization or localization of the prion by the peptide reagent. The prion-binding reagent and the “captured” prion protein typically form a complex that can be detected by methods that are further described herein. Frequently, the detection of the complex is accomplished by the use of a detection reagent. The detection reagent is typically a prion-binding reagent and is usually detectably labeled (or labelable, e.g., in the case of a primary detection antibody and a labeled secondary antibody).

The surrogate controls of the invention comprise a first domain that binds to the prion-binding reagent of a prion assay. For example, in one aspect, the first domain binds to a peptide reagent that interacts preferentially with PrP^{Sc}. The surrogate control may also comprise one or more detectable labels such that binding of the surrogate control to the prion-binding reagent (e.g., peptide reagent) can be readily detected.

In one aspect, the surrogate controls are bifunctional (or, in some cases, trifunctional) in that they comprise the first prion-binding reagent domain and a second domain, the second domain comprising a molecule that binds to the detection reagent of the prion assay. For example, if the detection reagent comprises an antibody, the second domain may comprise an epitope (or mimotope) recognized by the antibody. Alternatively, the second domain and detection reagent may each comprise one member of a binding pair of molecules (e.g., biotin and streptavidin, etc.). Thus, the first and second domains are typically different molecules from each other but may, in some cases, may be the same molecule.

The second domain of a bifunctional surrogate may bind directly to a detectably labeled detection reagent. Alternatively, the second domain may recognize a component of a detection system. For example, in certain immunoassays (such as ELISA), the analyte (e.g., prion or surrogate control) is detected by binding to a primary antibody, which primary antibody in turn binds to a detectably labeled secondary antibody. Thus, in certain embodiments, the second domain recognizes a primary antibody of a two-antibody detection reagent system.

Bifunctional (or trifunctional) surrogate controls of the invention may be single molecules (e.g., a fusion or chimeric protein comprising two domains) or two (or more) separately synthesized molecules that are subsequently covalently or non-covalently linked to each other. The molecules may be linked in any manner known in the art provided that the binding functions of the domains are preserved. Bifunctional surrogate controls comprising two domains may also comprise one or more linkers between the two domains.

Bifunctional surrogate controls described herein are advantageously used in prion detection assays employing as the prion-binding reagent one or more peptide reagents that interact preferentially with PrP^{Sc}. Many anti-PrP antibodies and the PrP epitopes that they recognize are known, for example as set forth in Table A.

TABLE A

PrP Antibodies and Epitopes

| | | Material | |
|---|---|---|---|
| Ab | Epitope/Immunogen Peptide | Source | Reference |
| 3F4 | PrP amino acids 109-112 | Chemicon | Prusiner, S. B., Science |
| | Human - MKEM | Sigma | 252, 1515 (1991) |
| | (SEQ ID NO: 261) | | |
| D18 | 133-157 of the hamster prion protein | InPro | Peretz et al., J. Mol. |
| | AMSRPMMHFGNDWEDRYYRENMNRY | | Biol., 273: 614-622 |
| | (SEQ ID NO: 262) | | |
| D13 | 96-106 of the hamster prion protein | InPro | 1) Peretz et al., J. Mol. |
| | HNQWNKPSKPK | | Biol., 273: 614-622, |
| | (SEQ ID NO: 263) | | 1997. |
| | | | 2) Peretz et aL, Nature, |
| | | | 412: 739-743, 2001. |
| | | | 3) Bosque et al., Proc. |
| | | | Natl. Acad. Sci., 99: |
| | | | 3812-3817, 2002. |
| | | | 4) Leclerc et al., J. Mol. |
| | | | Biol., 326: 475-483, |
| | | | 2003. |
| 6H4 | Murine PrP 143-151 | Prionics | Prionics, |
| | DWEDRYYRE | | Liu et al., J. |
| | (SEQ ID NO: 264) | | Histochemistry & |
| | | | Cytochemistry 51(8) |
| | | | 1065-1071, 2003 |
| MAB5 | Immunogen: Recombinant PrP amino acids | Chemicon | |
| 424 | 23-237. | | |
| 7D9 | Immunogen: Recombinant mouse PrP (amino | Biodesign | Kascsak, et al., (1987) |
| | acids 23-237) | Interna- | J. Virol., 61: 3688- |
| | | tional | 3693. |
| BDI115 | PrP peptide (a.a. 146-159 of bovine prion | Biodesign | Biodesign International |
| | protein) | Internat'l | |
| | NDYEDRYYRENMHR | | |
| | (SEQ ID NO: 269) | | |
| SAF32 | N-terminal octo-repteat region | SPI Bio | SPI Bio |
| SAF53 | PrP amino acids 142-160 (human numbering). | SPI Bio | SPI Bio |
| | GSDYEDRYYRENMHRYPNQ | | |
| | (SEQ ID NO: 270) | | |
| SAF83 | No Continuous Epitope Known | SPI Bio | SPI Bio |
| SAF84 | PrP amino acids 160-170 (human numbering). | SPI Bio | SPI Bio |
| | QVYYRPMDEYS | | |
| | (SEQ ID NO: 271) | | |
| 19B10 | Conformation specificity for NtmPrP | | WO2004033628A2 |
| 7VC | Copper-dependent specificity to CtmPrP | | WO2004033628A2 |
| 12F10 | Human 142-160 | SPI Bio | SPI Bio |
| | GSDYEDRYYRENMHRYPNQ | | |
| | (SEQ ID NO: 270) | | |
| PRI308 | PrP amino acids 106-126 (human numbering) | SPI Bio | SPI Bio |
| | KTNMKHMAGAAAAGAVVGGLG | | |
| | (SEQ ID NO: 272) | | |
| 34C9 | Bovine 138-142 | Prionics | Prionics |
| | LIHFG | | |
| | (SEQ ID NO: 273) | | |
| Fab | 96-105 of the hamster prion protein | InPro | 1) Bosque et al., Proc. |
| HuM-P | HNQWNKPSKP | | Natl. Acad. Sci., 99: |
| | (SEQ ID NO: 263) | | 3812-3817, 2002. |
| | | | 2) Safar et al., Nature |
| | | | Biotech., 20: 1147-50, |
| | | | 2002. |
| Fab | 226-231 of the Syrian hamster prion protein | InPro | 1) Peretz et al., Nature, |
| HuM- | YYDGRRS | | 412: 739-743, 2001 |
| R1 | (SEQ ID NO: 274) | | 2) Peretz et al., J. Mol. |
| | | | Biol., 273: 614-622, |
| | | | 1997. |
| | | | 3) Williamson et al., J. |
| | | | Virol., 72: 9413-9418 |
| | | | 4) Matsunaga et al., |
| | | | Proteins, 44: 110-118, |
| | | | 2001 |
| | | | 5) Leclerc et al., J. Mol. |
| | | | Biol., 326: 475-483, |
| | | | 2003 |
| Fab | 152-163 of the hamster prion protein | InPro | 1) Williamson et al., J. |
| HuM- | ENMNRYPNQVYY | | Virol., 72: 9413-9418, |
| R72 | (SEQ ID NO: 275) | | 1998. |
| | | | 2) Peretz et al., J. Mol. |
| | | | Biol., 273: 614-622, |
| | | | 1997. |
| | | | 3) Matsunaga et al., |
| | | | Proteins, 44: 110-118, |
| | | | 2001. |
| Mouse | Recognizes a conserved epitope (QYQRES) | VMRD, | Spraker et al. J. Vet. |
| Anti- | (SEQ ID NO:276) on the ruminant prion | Inc. | Diagn. Invest. 14(1): 3- |
| Prion | protein in tissues from sheep, cattle, mule | | 7 (2002) |
| Protein | deer, elk and white-tailed deer. | | |

In addition to the antibodies and epitopes listed above, antibodies generated against peptide reagents as described herein, fragments of these antibodies, or epitopes or mimotopes of these antibodies may also be used in the surrogate controls of the invention.

As noted above, the first and second domains of the surrogate controls are selected depending on the prion-binding reagent and detection reagents to be used in the assay. Tables B, C and D provide non-limiting examples of exemplary surrogate controls. In particular, Table B shows exemplary surrogate controls when the prion-binding reagent of the assay is a peptide reagent as described herein and the first domain recognizes the peptide reagent.

TABLE B

Bifunctional Surrogate Controls for Use with Peptide Reagent
Immunoassays

| | 1^{st} Domain | | |
|---|---|---|---|
| | Surrogate | 2nd Domain of | Detection |
| Peptide Reagent | Control | Surrogate Control | Reagent |
| PrP Pulldown | Antibody, Aptamer, | Epitope peptide or | Antibody |
| Peptide | protein, etc. that | mimotope to the | recognizing |
| | Recognizes PrPSc | assay+s primary | PrP |
| | Pulldown Peptide | antibody | |
| QWNKPSKPKTN | D13 | D18 Epitope Peptide | D18 |
| (SEQ ID NO: 14) | | AMSRPMMHFGNDW | |
| | | EDRYYRENMNRY | |
| | | (SEQ ID NO: 262) | |
| QWNKPSKPKTN | D13 | 6H4 Epitope Peptide | 6H4 |
| (SEQ ID NO: 14) | | DWEDRYYRE | |
| | | (SEQ ID NO: 264) | |
| QWNKPSKPKTN | 3F4 | D18 Epitope Peptide | D18 |
| MKHMGGG | | AMSRPMMHFGNDW | |
| (SEQ ID NO: 198) | | EDRYYRENMNRY | |
| | | (SEQ ID NO: 262) | |
| QWNKPSKPKTN | 3F4 | 6H4 Epitope Peptide | 6H4 |
| MKHMGGG | | DWEDRYYRE | |
| (SEQ ID NO: 198) | | (SEQ ID NO: 264) | |

Table C shows exemplary surrogate controls where the prion-binding reagent of the assay comprises a peptide reagent as described herein and the first domain recognizes an auxiliary motif on the peptide. The auxiliary motif may be, for example, a detectable label, a member of binding pair (e.g., biotin, His-6), etc., peptide that can be recognized independent of PrP pull-down peptide sequence. The first domain of the surrogate comprises a molecule that recognizes the auxiliary motif of the peptide reagent, for example, an antibody (or fragment thereof), an aptamer, a protein, etc.

TABLE C

Bifunctional Surrogate Controls for Use with
Peptide Reagents-Auxiliary Motif Immunoassays

| | Auxiliary | 1^{st} Surrogate | | Detection |
|---|---|---|---|---|
| Peptide Reagent | Motif | Domain | 2^{nd} Surrogate Domain | Reagent |
| PrP Pull-down | Biotin, His-6, | Antibody, | Epitope peptide or | Antibody |
| Peptide | peptide, etc. | aptamer, | mimotope to the assay's | recognizing |
| | recognized | protein, etc. | primary antibody | PrP |
| | independent | that recognizes | | |
| | of PrP pull- | the auxiliary | | |
| | down peptide | motif on the | | |
| | sequence | pull-down peptide | | |
| QWNKPSKPKTN | Biotin | Anti-Biotin | D18 Epitope Peptide | D18 |
| (SEQ ID NO: 14) | | | AMSRPMMHFGNDW | |
| | | | EDRYYRENMNRY | |
| | | | (SEQ ID NO: 262) | |
| QWNKPSKPKTN | Biotin | Anti-Biotin | 6H4 Epitope Peptide | 6H4 |
| (SEQ ID NO: 14) | | | DWEDRYYRE | |
| | | | (SEQ ID NO: 264) | |
| GGGKKRPKPGG | Biotin | Anti-Biotin | D18 Epitope Peptide | D18 |
| (SEQ ID NO: 68) | | | AMSRPMMHFGNDW | |
| | | | EDRYYRENMNRY | |
| | | | (SEQ ID NO: 262) | |
| GGGKKRPKPGG | Biotin | Anti-Biotin | 6H4 Epitope Peptide | 6H4 |
| (SEQ ID NO: 68) | | | DWEDRYYRE | |
| | | | (SEQ ID NO: 264) | |
| QWNKPSKPKTN | Biotin | Streptavidin | D18 Epitope Peptide | D18 |
| (SEQ ID NO: 14) | | | AMSRPMMHFGNDW | |
| | | | EDRYYRENNNRY | |
| | | | (SEQ ID NO: 262) | |
| QWNKPSKPKTN | Biotin | Streptavidin | 6H4 Epitope Peptide | 6H4 |
| (SEQ ID NO: 14) | | | DWEDRYYRE | |
| | | | (SEQ ID NO: 264) | |
| GGGKKRPKPGG | Biotin | Streptavidin | D18 Epitope Peptide | D18 |
| (SEQ ID NO: 68) | | | AMSRPMMHFGNDW | |
| | | | EDRYYRENMNRY | |
| | | | (SEQ ID NO: 262) | |
| GGGKKRPKPGG | Biotin | Streptavidin | 6H4 Epitope Peptide | 6H4 |
| (SEQ ID NO: 68) | | | DWEDRYYRE | |
| | | | (SEQ ID NO: 264) | |

Table D depicts exemplary surrogate controls in which the first domain comprises an epitope recognized by the antibody used as the prion-binding reagent in the assay. The second surrogate domain in turn comprises an epitope recognized by the detection reagent (antibody that recognized PrP).

TABLE D

Bifunctional Surrogate Controls for Use with Prion
Immunoassays

| Prion- | | | |
|---|---|---|---|
| Binding | 1st Surrogate | | Detection |
| Reagent | Domain | 2nd Surrogate Domain | Reagent |
| PrP-Ab | Epitope Peptide of | Epitope Peptide to | PrP-Ab |
| | Prion-binding | Primary Antibody | |
| | antibody | | |
| 3F4 | 3F4 Epitope | D18 Epitope Peptide | D18 |
| | Peptide MKHM | AMSRPMMHFGNDWEDRYYRENMNRY | |
| | (SEQ ID NO: 261) | (SEQ ID NO: 262) | |
| D13 | D13 Epitope | D18 Epitope Peptide | D18 |
| | Peptide | AMSRPMMHFGNDWEDRYYRENMNRY | |
| | HNQWNKPSKPK | (SEQ ID NO: 262) | |
| | (SEQ ID NO: 263) | | |
| 3F4 | 3F4 Epitope | 6H4 Epitope Peptide | 6H4 |
| | Peptide MKHM | DWEDRYYRE | |
| | (SEQ ID NO: 261) | (SEQ ID NO: 264) | |

In any of the surrogate controls described herein, one or more domains may include multiple recognition sites. When the detection method utilizes a two-antibody sandwich type ELISA, the surrogate control will comprise a third domain, which third domain binds to the “recapture” antibody. For example, if the SAF32 antibody is used for recapture of the dissociated pathogenic prion protein and the 3F4 antibody is used as the detection antibody, the surrogate control will comprise the recognition epitopes for both the SAF32 and the 3F4 antibodies in addition to a domain that binds to the peptide reagent used in the “pull-down” step.

### VI. ADDITIONAL APPLICATIONS

### A. Detection

As described above, the method of detecting pathogenic prion proteins described herein can be used to diagnose prion disease in a subject. In addition, the method described above can also be used to detect pathogenic prion contamination in blood and/or food supplies. Thus, a blood supply can be prepared that is substantially free of pathogenic prions by screening aliquots from individual collected samples or pooled samples using any of the methods of detection described herein. Samples or pooled samples that are contaminated with pathogenic prions can be eliminated before they are combined. In this way, a blood supply substantially free of pathogenic prion contamination can be provided. By “substantially free of pathogenic prions” is meant that the present of pathogenic prions is not detected using any of the assays described herein. Importantly, the peptide reagents described herein, which have already been shown to detect pathogenic protein forms in brain tissue diluted 10⁶ fold by normal tissue, are the only demonstrated reagent that may be capable of detecting pathogenic prions in blood.

The invention thus provides a method of preparing blood supply that is substantially free of pathogenic prions, said blood supply comprising whole blood, red blood cells, plasma, platelets or serum, said method comprising: (a) screening aliquots of whole blood, red blood cells, plasma, platelets or serum from collected blood samples by any of the detection methods provided herein for detecting; and (b) combining only samples in which pathogenic prions are not detected to provide a blood supply that is substantially free of pathogenic prions.

Similarly, the food supply can be screened for the presence of pathogenic prions in order to provide food that is substantially free of pathogenic prions. Thus, using any of the methods described herein, samples from live organisms intended as food for human or animal consumption can be screened for the presence of pathogenic prions. Samples taken from food product intended to enter the food supply can also be screened. Samples in which pathogenic prions are detected are identified and the live organism or food intended to enter the food supply from which the samples in which pathogenic prions were detected are removed from the food supply. In this way, a food supply that is substantially free of pathogenic prions can be provided.

The invention thus provides a method of preparing food supply that is substantially free of pathogenic prions, said method comprising: (a) screening a sample collected from live organisms that will enter the food supply or a sample collected from food intended to enter the food supply by any of the detection methods provided herein for detecting pathogenic prions; and (b) combining only samples in which pathogenic prions are not detected to provide a food supply that is substantially free of pathogenic prions.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Peptide Reagent Production

Peptide fragments of prion proteins were chemically synthesized using standard peptide synthesis techniques, essentially as described in Merrifield (1969) Advan. Enzymol. 32: 221 and Holm and Medal (1989), Multiple column peptide synthesis, p. 208E, Bayer and G. Jung (ed.), Peptides 1988, Walter de Gruyter & Co. Berlin-N.Y. Peptides were purified by HPLC and sequence verified by mass spectroscopy.

In certain cases, the peptides synthesized included additional residues at the N or C terminus, for example GGG residues and/or included one or more amino acid substitutions as compared to wild-type sequences.

### A. Peptoid Substitutions

Peptoid substitutions were also made in the peptide presented in SEQ ID NO:14 (QWNKPSKPKTN, corresponding to residues 97 to 107 of SEQ BD NO:2), SEQ ID NO:67 (KKRPKPGGWNTGG, corresponding to residues 23-36 of SEQ ID NO:2) and SEQ ID NO:68 (KKRPKPGG, corresponding to residues 23-30 of SEQ ID NO:2). In particular, one or more proline residues of these peptides were substituted with various N-substituted peptoids. See, FIG. 3 for peptoids that can be substituted for any proline. Peptoids were prepared and synthesized as described in U.S. Pat. Nos. 5,877,278 and 6,033,631, both of which are incorporated by reference in their entireties herein; Simon et al. (1992) Proc. Natl. Acad. Sci. USA 89:9367.

### B. Multimerization

Certain peptide reagents were also prepared as multimers, for example by preparing tandem repeats (linking multiple copies of a peptide via linkers such as GGG), multiple antigenic peptides (MAPS) and/or linearly-linked peptides.

In particular, MAPS were prepared using standard techniques, essentially as described in Wu et al. (2001) J Am Chem. Soc. 2001 123(28):6778-84; Spetzler et al. (1995) Int J Pept Protein Res. 45(1):78-85.

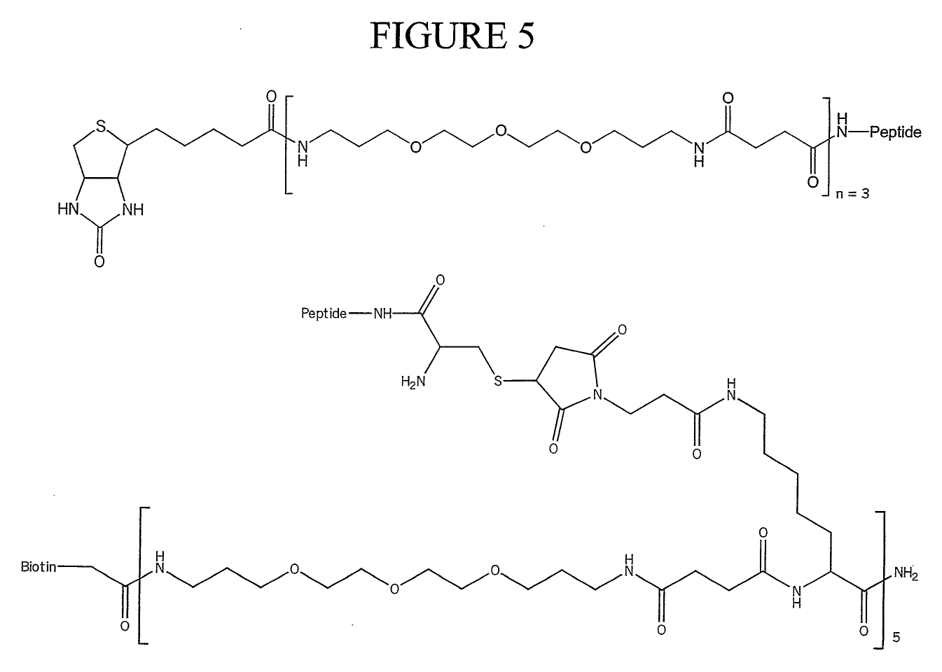

Linear and branched peptides (e.g., PEG linker multimerization) were also prepared using polyethylene glycol (PEG) linkers, using standard techniques. In particular, branched multipeptide PEG scaffolds were created with the following structures: Biotin-PEG-Lys-PEG-Lys-PEG-Lys-PEG-Lys-PEG-Lys (no peptide control) and Biotin-PEG-Lys(Peptide)-PEG-Lys(Peptide)-PEG-Lys(Peptide)-PEG-Lys(Peptide)-PEG-Lys(Peptide). In addition, peptide to Lys linkages were prepared: Lys-epsilon-NH—CO—(CH₂)₃-Mal-S-Cys-peptide. See, FIG. 5

### C. Biotinylation

Peptides were biotinylated using standard techniques following synthesis and purification. Biotin was added to the N- or C-terminal of the peptide.

### Example 2

### Binding Assays

### A. Pull-Down

Peptide reagents as described herein were tested for their ability to specifically bind to prion proteins using a magnetic bead pull down assay. For this assay, the peptide reagents were either labeled with biotin, which allowed attachment to streptavidin coated magnetic beads or were covalently attached to magnetic beads.

Brain homogenates are prepared from RML PrP^{Sc+} and PrP^{C+} Balb-c mice. In brief, 5 mL of TBS buffer (50 mM Tris-HCl pH 7.5 and 37.5 mM NaCl) with 1% TW20 and 1% triton 100 was added to brains weighing ˜0.5 g to produce a 10% homogenate. The brain slurry was dounced until large particles had disappeared. Aliquots of 200 μl were diluted 1:1 in buffer were added to pre-cooled eppendorf tubes and the samples sonicated for several repeats of several seconds each. Samples were centrifuged for 10-15 minutes at. 500× and the supernatants removed.

To test the effect of Proteinase K digestion, certain supernatants were divided into two samples and 4 μl of Proteinase K was added to one sample and rotated at 37° C. for 1 hour. Eight microliters of PMSF was added to the proteinase K tubes to stop digestion and the tubes were incubated for a minimum of 1 hour at 4° C.

In addition, different formats of pull-down with biotinylated peptides and streptavidin magnetic beads were also tested. In a first set of experiments, infectious brain homogenate was mixed with biotinylated peptides and streptavidin beads added subsequently. In a second set of experiments, magnetic streptavidin beads were coated with biotinylated peptides and then mixed with infectious brain homogenates. In both set of experiments, following incubation of all three components (beads, peptides and brain homogenates) together, the mixtures were washed and treated with 3 M GdnSCN for 15 minutes at room temperature and ELISA assays performed as described below.

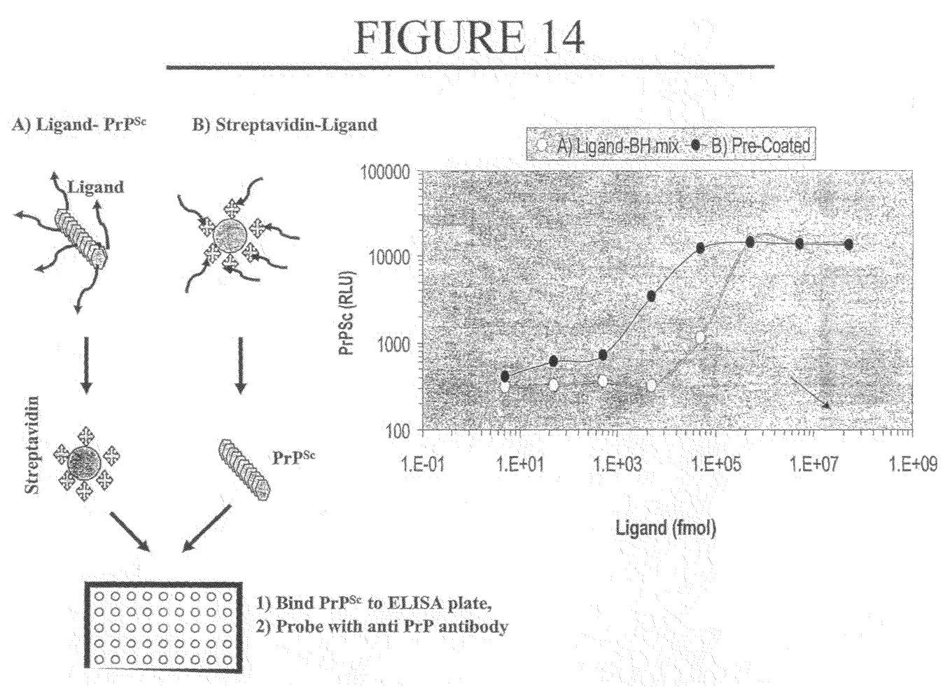

As shown in FIG. 14, the second format (beads coated with biotinylated peptides prior to mixing with brain homogenates) was approximately 100 times more efficient at isolating (pulling-down) PrP^{Sc} than the first format (beads added after biotinylated peptides were mixed with brain homogenates). Based on these results, further detection experiments were conducted following the second format.

Homogenates were stored at 4° C. degrees until further use and sonicated again as described above if needed. A 10% w/v PrP^{C+} or PrP^{Sc+} preparation of the brain homogenates was incubated overnight at 4° C. with a biotin-labeled peptide reagent, as follows Tubes containing 400 μl of buffer, 50 μl of extract and 5 μl of biotin-labeled peptide reagent (10 mM stock) were prepared. The tubes were incubated for a minimum of 2 hours at room temperature or overnight at 4° C. on platform rocker.

Following incubation, 50 μl of SA-beads (Dynal M280 Streptavidin 112.06) were added and the tubes mixed by vortexing. The tubes were incubated, with rocking (VWR, Rocking platform, Model 100), for 1 hour at room temperature or overnight in at 4° C.

Samples were removed from shaker, placed in magnetic field to collect the magnetic beads with attached peptide reagent and prion and washed 5-6 time using 1 ml assay buffer. Samples were used immediately or stored at −20° C. until Western blotting or ELISA, described below.

### B. Western Blotting

Western blotting analysis was performed as follows. Bead-peptide-prion complexes precipitated as described above were denatured after the final wash by adding 25-30 μl of SDS buffer (Novex Tris-Glycine SDS-Sample Buffer 2×) added to each tube. The tubes were mixed by vortexing until all of the beads were suspended. The tubes were boiled until the tops started to come open, run on a standard SDS-PAGE gel and transferred to a solid membrane for WB analysis.

The membrane was blocked for 30 minutes in 5% Milk/TBS-T [50 ml 1 M Tris pH 7.5; 37.5 ml 4 M NaCl, 1-10 mL Tween bring volume to 1 L with milk] at room temperature. Between 10-15 ml of anti-prion polyclonal antibodies, as described in International Application No. PCT/US03/31057, filed Sep. 30, 2003, entitled “Prion Chimeras and Uses Thereof”, which application is incorporated by reference herein, were added at a 1:50 fold dilution to the membrane and incubated for 1 hour at room temperature. The membrane was washed multiple times in TBS-T. After washing, the secondary antibody (goat anti-rabbit IgG (H+L) antibody (Pierce) conjugated to alkaline phosphatase (AP) was added at 1:1000 dilution (in TBS-T) and incubated for 20 minutes at room temperature. The membrane was washed multiple times in TBS-T. Alkaline phosphatase precipitating reagent (1-step NBT/BCIP (Pierce) was added and developed until background appeared or signal was apparent.

### C. ELISA

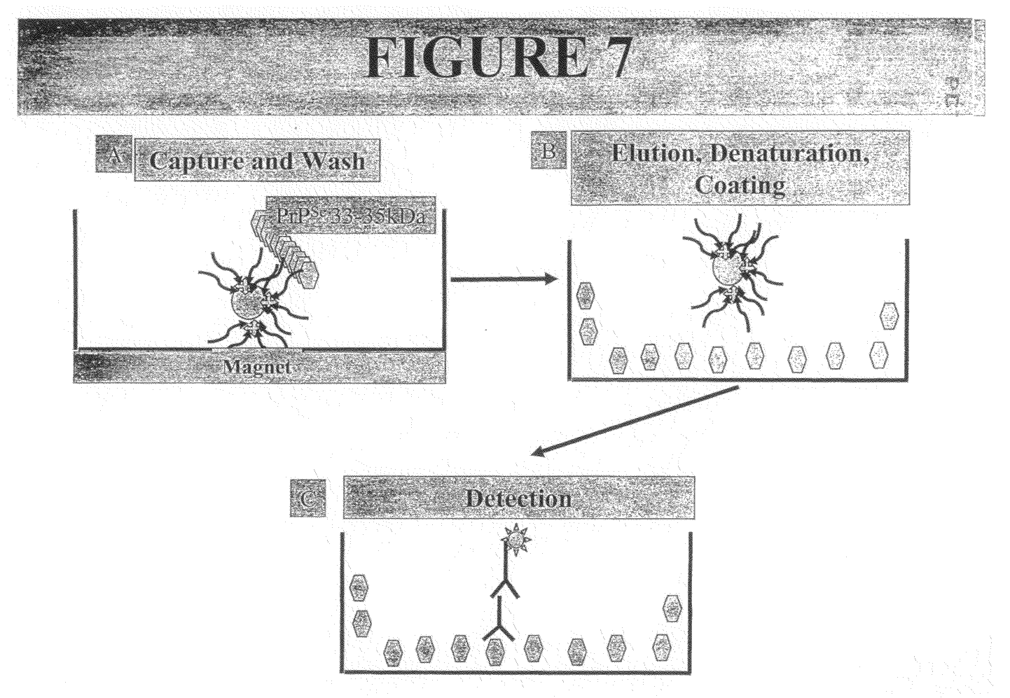

Indirect ELISA was performed as follows. (FIG. 7). (Indirect ELISA uses antigen-coated plates, in this case plates coated with PrP from the pull-down step, with a unlabeled primary antibody specific for the antigen and a labeled secondary antibody that binds to the primary antibody). Pull-down of PrP^{Sc} in various samples was performed as described above. Briefly, magnetic beads were coated with one or more peptide reagents as described herein and aliquoted into a 96-well plate. Samples of mouse brain homogenates, human plasma spiked with PrP^{Sc}, Syrian hamster (SHa) brain homogenates from normal and scrapie brains, human vCJD brain, and brain homogenates from normal and diseased (CWD PrP^{Sc}) mice transgenic for the deer PrP gene were incubated with the peptide reagent-coated beads for 4 hours at room temperature, to allow any PrP^{Sc} in the sample to bind to the peptide reagent-coated beads.

Following capture of PrP^{Sc} by the peptide reagent beads, the wells were washed to remove unbound proteins by exposing the plates to a magnetic field and removing the supernatant. The PrP^{Sc} bound to the peptide was then dissociated from the peptide bead. Because no antibodies are yet available that recognize native (non-denatured) PrP^{Sc}, the PrP^{Sc} was dissociated under denaturing conditions, i.e., by incubation with 3M or 6M guanidine thiocyanate (GdnSCN). See, e.g., Peretz et al. (1997) J. Mol. Biol. 273(3):614-622; Ryou et al. (2003) Lab Invest. 83(6):837-43. Dissociated PrP^{Sc} was coated onto the plates by incubation with 0.1M NaHCO₃, pH 8.9 (110 μl/well) and the beads removed from the wells by aspiration and washing (3× with 200 μl TBS with 0.05% TW20).

Following washing, the wells (coated with any PrP^{Sc} in the sample) were blocked with 200 μl of 3% BSA in TBS for 1 hour at 37° C. The blocking solution was then aspirated out of the wells and 100 μl of 0.5 μg/ml solution of primary Fab D18 (Peretz et al. (2001) Nature 412(6848):739-743) in TBS with 1% BSA was added to each well and incubated for 2 hours at 37° C. The wells were then washed 9× with 300 μl of TBC with 0.05% TW2. Goat anti-human antibody conjugated with alkaline phosphatase (AP) was added to each well (100 μl of a 1:5000 dilution) and the plate incubated for 1 hour at 37° C. After washing (9× with 300 μl of TBC with 0.05% TW2), 100 μl of AP substrate was added to each well, the plates incubated at 37° C. for 0.5 hours and the optical density (OD) of the plates read.

Indirect ELISA results are shown in Table 2 and FIGS. 7 to 12. Table 2 shows O.D. values for various peptide reagents. O.D. values over blank controls (ranging from 0.172-0.259) were considered positive.

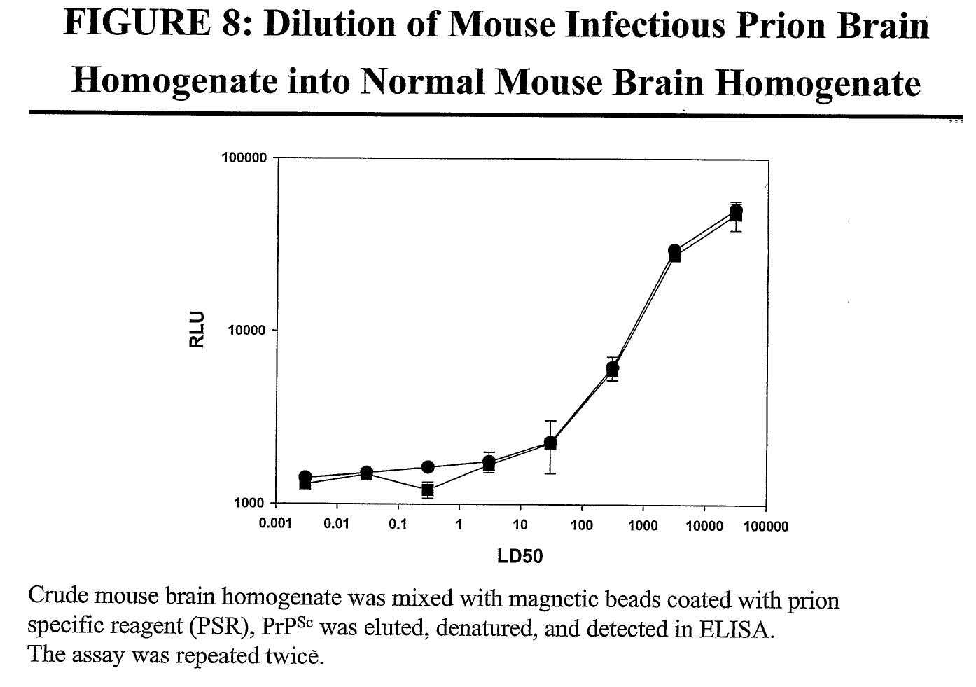

FIG. 8 shows ELISA detection of PrP^{Sc} from mouse brain homogenates spiked with infectious prions particles at various dilutions. ELISA assays were performed as described above. LD₅₀ is defined as the lethal dose of PrP^{Sc} that will kill 50% of animals, and has been determined for many rodent models including mice. See, e.g., Klohn et al. (2003) Proc Natl Acad Sci USA 100(20):11666-11671. The ELISA assay detected lower than 100 LD₅₀ units of prion infectivity in plasma and buffy coat, the sensitivity required for detecting prions in blood samples.

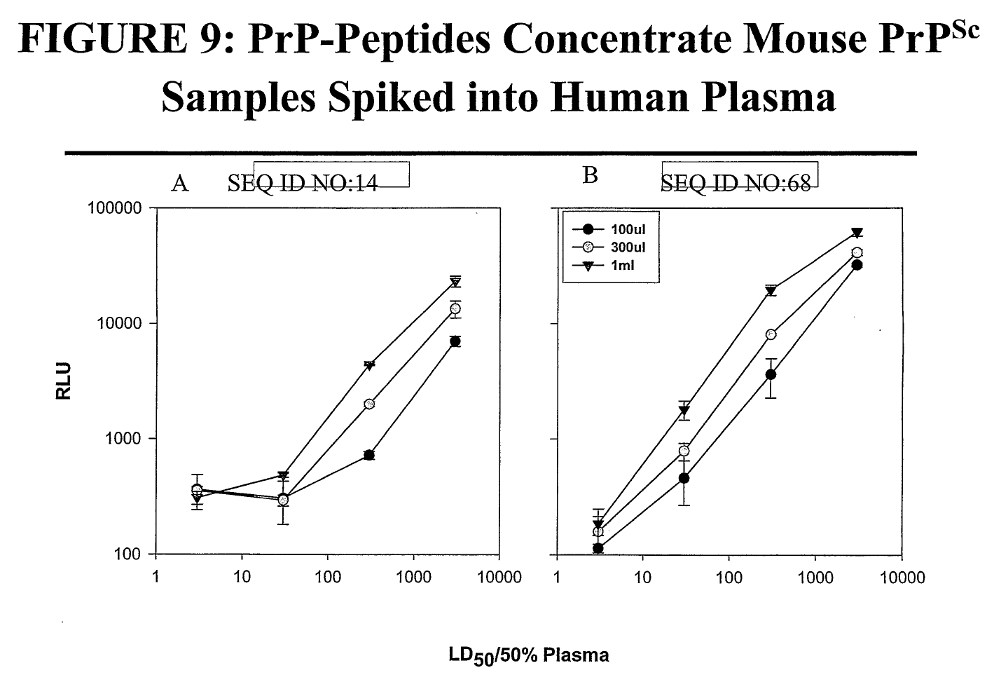

FIG. 9 shows ELISA results of mouse PrP^{Sc} spiked into human plasma using QWNKPSKPKTN-biotin (SEQ ID NO:14) (FIG. 9A) and biotin-GGGKKRPKPGG (SEQ ID NO:68) (FIG. 9B) as capture (pull-down) reagents.

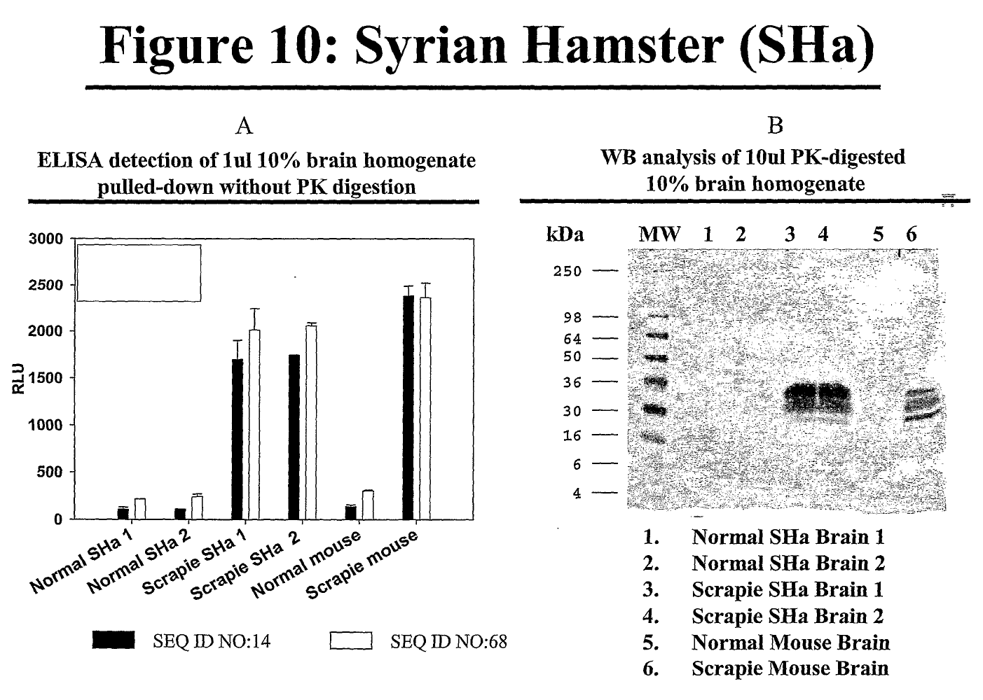

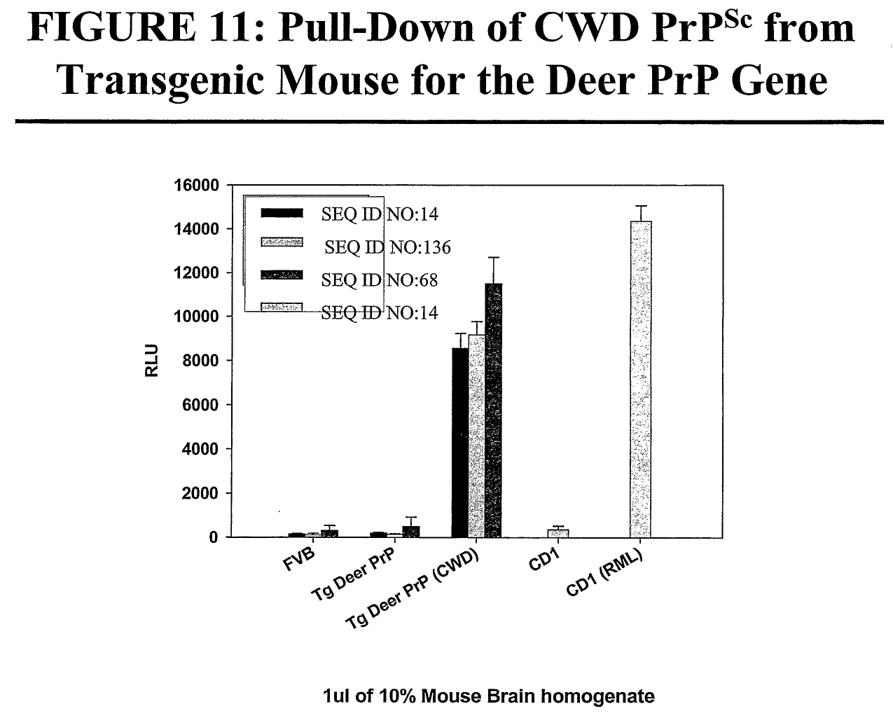

FIG. 10A shows ELISA detection of 1 μl 10% brain homogenates of normal and PrP^{Sc}-infected Syrian Hamsters (SHa) (purchased from VA Medical Center, Baltimore, Md.) pulled down using QWNKPSKPKTN-biotin (SEQ ID NO:14) and biotin-GGGKKRPKPGG (SEQ ID NO:68) and without Proteinase K (PK) digestion. FIG. 10B shows Western Blot analysis of samples subject to PK-digestion. FIG. 11 shows ELISA detection of PrP^{Sc} in transgenic mice carrying the deer PrP gene (obtained from Glenn Telling, University of Kentucky, see, Browning et al. (2004) J. Virol. 78(23):13345-13350). PrP^{Sc} was pulled down using QWNKPSKPKTN-biotin (SEQ ID NO:14), biotin-KKKAGAAAAGAVVGLGG-CONH2 (SEQ ID NO:136), and GGGKRPKPGG (SEQ ID NO:68) and detected by ELISA as described above.

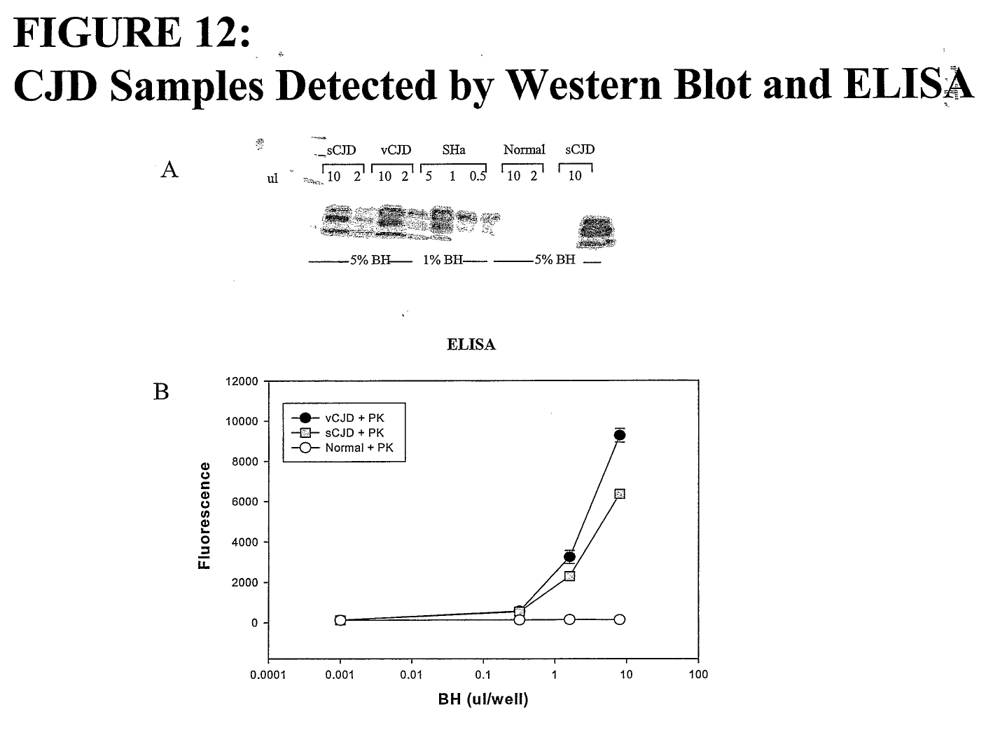

FIG. 12 shows detection of PrP^{Sc} in various CJD samples by Western blot (FIG. 12A) and ELISA (FIG. 12B).

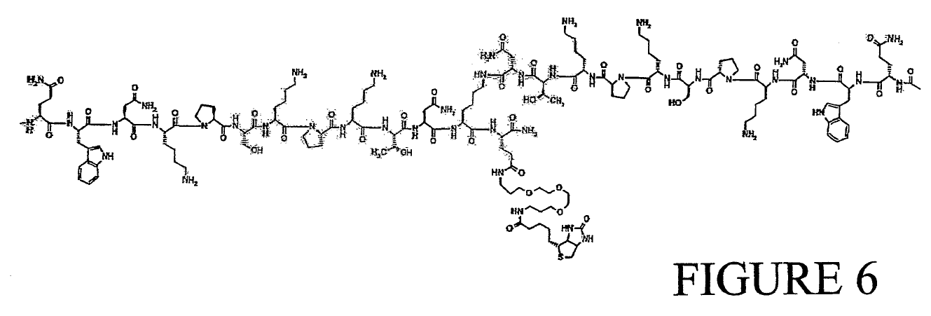

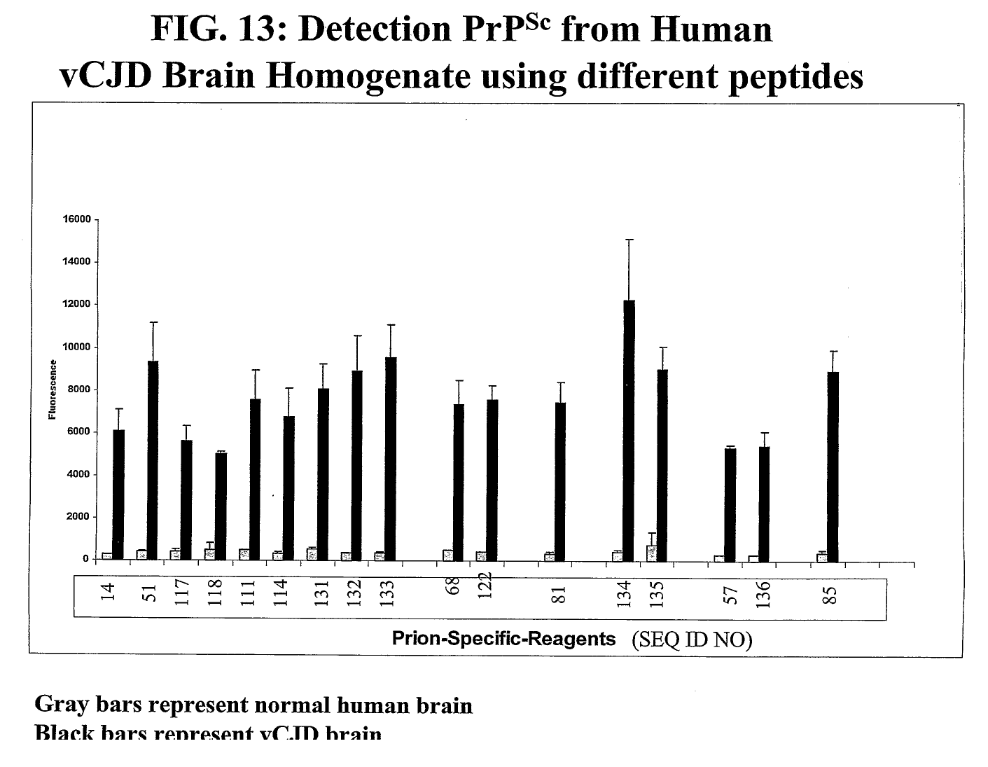

FIG. 13 shows detection of PrP^{Sc} in vCJD brain homogenates using various peptides described herein as follows: QWNKPSKPKTN-biotin (SEQ ID NO:14); QWNKPSKPTKTNGGGQWNKPSKPKTN-biotin (SEQ ID NO:51); biotin-QWNKPSKPKTN, where P5 is substituted with N-(3,5-dimethoxybenzyl)glycine (SEQ ID NO: 117); biotin-QWNKPSKPKTN, where P5 is substituted with N-butylglycine (SEQ ID NO: 118); biotin-QWNKPSKPKTN, where P8 is substituted with N-(cyclopropylmethyl)glycine (SEQ ID NO:111); biotin-QWNKPSKPKTN, where P8 is substituted with N-butylglycine (SEQ ID NO:114); biotin-QWNKPSKPKTN, where P5 is substituted with N-(cyclopropylmethyl)glycine and P8 is substituted with N-butylglycine (SEQ ID NO:131); biotin-QWNKPSKPKTN, where P5 is substituted with N-(isopropyl)glycine and P8 is substituted with N-(cyclopropylmethyl)glycine (SEQ ID NO:132); QWNKPSKPKTN2K-biotin (SEQ ID NO:133; FIG. 6); biotin-GGGKKRPKPGG (SEQ ID NO:68); biotin-KKRPKPGG, where P6 is substituted with N-(cyclopropylmethyl)glycine (SEQ ID NO:122); biotin-GGGKKRPKPGGGQWNKPSKPKTN (SEQ ID NO:81); 4-branchMAPS-GGGKKRPKPGGWNTGGG-biotin (SEQ ID NO:134); 8-branchMAPS-GGGKKRPKPGGWNTGGG-biotin (SEQ ID NO:135); biotin-KKKAGAAAAGAVVGGLGGYMLGSAM (SEQ ID NO:57); biotin-KKKAGAAAAGAVVGGLGG-CONH₂ (SEQ ID NO:136); and biotin-GGGKKKKKKKK (SEQ ID NO:85).

### D. Results

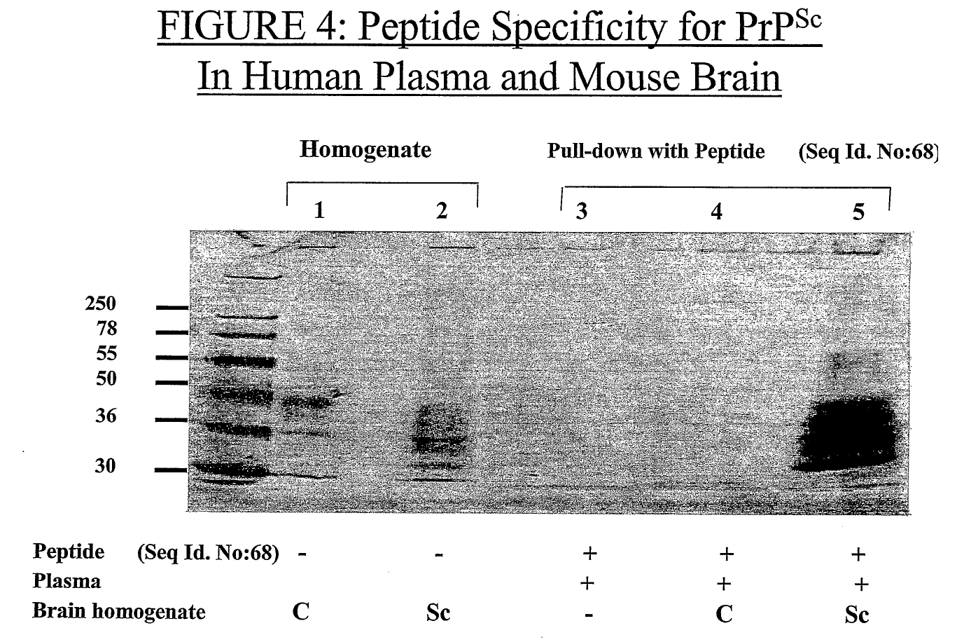

Results of Western blotting and indirect ELISA binding assays are summarized in Table 2 and FIGS. 8 through 14. In brief, proteinase K digestion of brain homogenates was not necessary in order to detect specific binding of the peptide reagents as described herein to bind to PrP^{Sc}. As shown in FIG. 4, in no case was binding observed to wild type brain homogenates, indicating that the peptide reagents were binding to PrP^{Sc} specifically. FIGS. 8-14 demonstrate the sensitivity and specificity across different species. Furthermore, Western blotting analysis described above detected PrP^{Sc} at over four logs dilution while ELISA was at least 10× more sensitive than Western blotting.

Thus, the peptide reagents described herein allow for a simple, one-well, high-throughput assay that efficiently detects the presence of PrP^{Sc} from various species in a biological sample at sensitivities lower than 100 LD₅₀ and without the need for proteinase K digestion.

TABLE 2

| Peptide reagent (biotin la- | | Western | ELISA |
|---|---|---|---|
| beled on N-or C-terminal) | Seq Id: | Blot¹ | A_{405 nm} |
| ³CGG⁵WGQGGGTHNQWNXPSKPKT | 35 | + | 0.687 |
| NLKHV³C | | | |
| ³GGWGQGGGTHNQWNKPSKPKTNLK | 36 | + | ND |
| V | | | |
| GGWGQGGGTHNQWNKPSKPKTNL | 37 | + | ND |
| KHV³ | | | |
| C⁵GGWGQGGGTHNQWNKPSKPKTN | 40 | + | ND |
| LKHV³C | | | |
| RPMIHFGNDWEDRYYRENMYR⁴ | 44 | − | ND |
| ⁴RPMIHFGNDWEDRYYRENMYR⁵C | 76 | − | ND |
| ⁵C⁴RPMIHFGNDWEDRYYRENMYR⁴ | 46 | + | ND |
| C² | | | |
| QWNKPSKPKTN⁴ | 50 | + | 0.932 |
| QWNKPSKPKTN | 14 | +++ | 0.775 |
| QWNKPSKPKTN⁴QWNKPSKPKTN | 51 | +++ | .923 |
| QWNKPSKPKTNLKHV⁴ | 77 | ++ | 0.839 |
| GGWGQGGGTHNQWNKPSKPKTN | 53 | + | 0.254 |
| GGTHNQWNKPSKPKTN | 54 | + | 0.253 |
| ⁴AGAAAAGAVVGGLGGYMLGSAM | 78 | insoluble | 0.259 |
| ⁴AGAAAAGAVVGGLGG | 56 | insoluble | 0.313 |
| ⁶AGAAAAGAVVGGLGGYMLGSAM | 57 | + | 0.901 |
| ⁶AGAAAAGAVVGGLGG | 65 | ++ | 0.635 |
| ⁴KKRPKPGGWNTGGSRYPGQGS | 66 | + | 0.533 |
| ⁴KKRPKPGGWNTGG | 67 | ++ | 0.451 |
| ⁴KKRPKPGG | 68 | +++ | 0.765 |
| PHGGGWGQPHGGSWGQPHGGSWG | 69 | − | 0.282 |
| Q | | | |
| PHGGGWGQPHGGSWGQ | 70 | − | 0.241 |
| PHGGGWGQ | 71 | − | 0.263 |
| ⁴GPKRKGPK | 73 | + | 1.0621 |
| ⁴WNKPSKPKT | 75 | − | 0.247 |
| ⁴NKPSKPK | 79 | − | 0.24 |
| ⁴KPSKPK | 80 | − | 0.225 |
| ⁴KKRPKPGGGKKRPKPGG | 72 | + | 0.522 |
| ⁴KKRPKPGGGQWNKPSKPKTN | 81 | + | 1.247 |
| KKKAGAAAAGAVVGGLGGYMLGS | 82 | − | 0.340 |
| AMDDD | | | |
| DDDAGAAAAGAVVGGLGGYMLGS | 83 | − | 0.237 |
| AM | | | |
| KKKAGAAAAGAVVGGLGGYMLGS | 84 | + | 0.268 |
| AMKKK | | | |
| ⁴KKKKKKKK | 85 | +³ | 0.530 |
| DDDAGAAAAGAVVGGLGGYMLGS | 86 | − | 0.227 |
| AMDDD | | | |
| ⁴NNKQSPWPTKK | 87 | − | 0.277 |
| DKDKGGVGALAGAAVAAGGDKDK | 88 | − | 0.282 |
| ⁴QANKPSKPKTN | 89 | + | 0.245 |
| ⁴QWNKASKPKTN | 90 | − | 0.283 |
| ⁴QWNKPSKAKTN | 91 | − | 0.256 |
| ⁴QWNAPSKPKTN | 92 | − | 0.230 |
| ⁴QWNKPSAPKTN | 93 | − | 0.250 |
| ⁴QWNKPSKPATN | 94 | − | 0.260 |
| ⁴QWNKASKAKTN | 95 | − | 0.241 |
| ⁴KKRAKPGG | 96 | + | 2.19 |
| ⁴KKRPKAGG | 97 | + | 1.24 |
| ⁴KKRAKAGG | 98 | + | 1.46 |

¹Visually evaluated relative signal intensity
²cyclized
³GGGG residues added/inserted at indicated position
⁴GGG residues added/inserted at indicated position
⁵GG residues added/inserted at indicated position
⁶KKK residues added/inserted at indicated position
ND = not determined

Alanine scanning was also performed to identify residues involved in binding.

Results are shown in Table 3.

TABLE 3

| Peptide reagent (biotin | | | |
|---|---|---|---|
| label on N- or C- | SEQ ID | Western | ELISA |
| terminus) | NO | Blot | A_{405 nm} |
| QWNKPSKPKTN | 14 | +++ | 0.775 |
| QANKPSKPKTN | 89 | +++ | 0.245 |
| QWNAPSKPKTN | 92 | + | 0.283 |
| QWNKPSAPKTN | 93 | + | 0.256 |
| QWNKPSKPATN | 94 | + | 0.230 |
| QWNKASKPKTN | 99 | +/− | 0.250 |
| QWNKPSKAKTN | 91 | + | 0.260 |
| QWNKASKAKTN | 95 | − | 0.241 |
| QWAKPSKPKTN | 100 | ND | 0.376 |
| QWNKPAKPKTN | 101 | ND | 0.356 |
| QWNKPSKPKAN | 102 | ND | 0.234 |
| QWNKPSKPKTA | 103 | ND | 0.262 |
| KKRPKPGG | 68 | +++ | 0.765 |
| AKRPKPGG | 104 | + | 0.273 |
| KARPKPGG | 105 | + | 0.256 |
| KKAPKPGG | 106 | + | 0.268 |
| KKRPAPGG | 107 | + | 0.578 |
| KKRAKPGG | 96 | ++ | 2.19 |
| KKRPKAGG | 97 | ++ | 1.24 |
| KKAPKAGG | 108 | + | 1.46 |

In addition, as shown in Table 4, binding to PrP^{Sc} by the peptide reagents having SEQ ID NO:14, SEQ ID NO:67 and SEQ ID NO:68 was further enhanced by substitutions at the proline residues by a number of N-substituted glycines (peptoids). See, also, FIG. 13.

TABLE 4

| | |
|---|---|
| Western | ELISA |
| Blot | A_{405 nm} |

| | | |
|---|---|---|
| * in (GGG)¹QWNKPSK*KTN | | |
| (SEQ ID NO: 14) | | |
| Proline | +++ | 0.775 |
| N-(S)-(1-phenylethyl)glycine (pep- | ++ | 0.865 |
| toid as circled in FIG. 3A) | | |
| (SEQ ID NO: 109) | | |
| N-(4-hydroxyphenyl)glycine (pep- | − | 0.934 |
| toid as circled in FIG. 3B) | | |
| (SEQ ID NO: 110) | | |
| N-(cyclopropylmethyl)glycine (pep- | +++++ | 1.141 |
| toid as circled in FIG. 3C) | | |
| (SEQ ID NO: 111) | | |
| N-(isopropyl)glycine (peptoid as | ND | 0.974 |
| circled in FIG. 3D) | | |
| (SEQ ID NO: 112) | | |
| N-(3,5-dimethoxybenzyl)glycine | +++ | 2.045 |
| (peptoid as circled in FIG. 3E) | | |
| (SEQ ID NO: 113) | | |
| N-amino butylglycine (peptoid as | ++++ | 0.776 |
| circled in FIG. 3F) | | |
| (SEQ ID NO: 114) | | |
| * in (GGG)¹QWNK*SKPKTN | | |
| (SEQ ID NO: 14) | | |
| N-(cyclopropylmethyl)glycine | ND | 0.498 |
| (SEQ ID NO: 115) | | |
| N-(isopropyl)glycine | ND | 1.57 |
| (SEQ ID NO: 116) | | |
| N-(3,5-dimethoxybenzyl)glycine | ND | 0.823 |
| (SEQ ID NO: 117) | | |
| N-amino butylglycine | ND | 0.619 |
| (SEQ ID NO: 118) | | |
| * in (GGG)¹KKRPK*GG | | |
| (SEQ ID NO: 68) | | |
| proline | ND | 0.765 |
| N-amino butylglycine | ND | 0.61 |
| (SEQ ID NO: 119) | | |
| N-(3,5-dimethoxybenzyl)glycine | ND | 0.631 |
| (SEQ ID NO: 120) | | |
| N-(isopropyl)glycine | ND | 0.509 |
| (SEQ ID NO: 121) | | |
| N-(cyclopropyhnethyl)glycine | ND | 0.503 |
| (SEQ ID NO: 122) | | |
| * in (GGG)¹KKRPK*GGWNTGG | | |
| (SEQ ID NO: 67) | | |
| Proline | ND | 0.451 |
| N-amino butylglycine | ND | 0.503 |
| (SEQ ID NO: 123) | | |
| N-(3,5-dimethoxybenzyl)glycine | ND | 0.464 |
| (SEQ ID NO: 124) | | |
| N-(isopropyl)glycine | ND | 0.555 |
| (SEQ ID NO: 125) | | |
| N-(cyclopropylmethyl)glycine | ND | 0.344 |
| (SEQ ID NO: 126) | | |
| (GGG)¹QWNKX1SKX2KTN | | |
| N-(cyclopropylmethyl)glycine at | ND | ND |
| X1; N-(cyclopropylmethyl)glycine | | |
| at X2 | | |
| (SEQ ID NO: 129) | | |
| N-(cyclopropylmethyl)glycine at | ND | ND |
| X1; N-(3,5-dimethoxybenzyl)glycine | | |
| at X2 | | |
| (SEQ ID NO: 130) | | |
| N-(cyclopropylmethyl)glycine at | ND | ND |
| X1; N-amino butylglycine at X2 | | |
| (SEQ ID NO: 131) | | |
| N-(isopropyl)glycine at X1; N- | ND | ND |
| (cyclopropylmethyl)glycine at X2 | | |
| (SEQ ID NO: 132) | | |
| N-(isopropyl)glycine at X1; N- | ND | ND |
| (3,5-dimethoxybenzyl)glycine at X2 | | |
| (SEQ ID NO: 257) | | |
| N-(isopropyl)glycine at X1; N- | ND | ND |
| amino butyiglycine at X2 | | |
| (SEQ ID NO: 258) | | |
| * in (GGG)¹KKR*KPGGWNTGG | | |
| (SEQ ID NO: 67) | | |
| N-amino butyLglycine | ND | ND |
| (SEQ ID NO: 249) | | |
| N-(3,5-dimethoxybenzyl)glycine | ND | ND |
| (SEQ ID NO: 250) | | |
| N-(isopropyl)glycine | ND | ND |
| (SEQ ID NO: 251) | | |
| N-(cyclopropylmethyl)glycine | ND | ND |
| (SEQ ID NO: 252) | | |
| * in (GGG)¹KKR*KPGG | | |
| (SEQ ID NO: 68) | | |
| N-amino butylglycine | ND | ND |
| (SEQ ID NO: 253) | | |
| N-(3,5-dimethoxybenzyl)glycine | ND | ND |
| (SEQ ID NO: 254) | | |
| N-(isopropyl)glycine | ND | ND |
| (SEQ ID NO: 255) | | |
| N-(cyclopropylmethyl)glycine | ND | ND |
| (SEQ ID NO: 256) | | |

¹The optional GGG linker was not present in the peptide reagents in the experiments shown in this table.

Furthermore, multimerization of PrP^{Sc}-binding peptide reagents also improved affinity for PrP^{Sc}. In particular, tandem repeats gave stronger signals (as measured by Western blotting) than single copies. Pre-derivatized MAP forms on beads increased binding in certain cases up to 2-fold. However, MAP forms caused precipitation of the peptide in solution. Linearly linked peptides were also tested for their ability to enhance binding without causing precipitation.

### Experiment 3

### Sandwich ELISA and pH Dissociation

Chaotropic agents like guanidinium salts are effective for dissociation of the PrP^{Sc} captured in the pull-down step and denaturation as shown in example 2. However, the guanidinium must be removed or significantly diluted in order to expose the denatured prion protein to anti-prion antibodies (that are used e.g., for detection of the PrP). This is not problematic for direct or indirect ELISA (in which the PrP is coated directly on the microtiter plate in a fairly large volume) but can be a problem for Sandwich ELISA. We have developed an alternative protocol for denaturation of the captured PrP^{SC} from the peptide reagent that does not use Gdn and does not require additional washes or introduction of large volumes for dilution. This method uses a pH treatment, at either high pH or low pH, for denaturation of the PrP^{SC}. The denatured PrP dissociates from the peptide reagent. The denaturing conditions can be easily removed by neutralizing the solution.

Sandwich ELISAs were carried out using 2 different anti-prion antibodies (one for “recapture” and one for detection) to detect PrP^{sc} after dissociation from the peptide reagent. These assays were carried out using either 3M GdnSCN or pH treatment at high or low pH to dissociate and denature the prion protein. The protocols for these experiments are outlined below.

Streptavidin magnetic beads (M-280 Dynabeads) were mixed with biotinylated peptide reagent having SEQ ID NO:68 and washed to remove unbound peptide reagent. The peptide-coated beads were used to pull down 0.025 μl of human vCJD 10% brain homogenate spiked into 100 μl solution containing 70% human plasma. After mixing for 1 h at 37° C., the beads were washed and treated with solutions of different pH. After 10 min incubation at room temperature, solutions were brought to neutral pH of about 7. The supernatants, containing the dissociated and denatured prion protein, were added to a microtiter plate that had been previously coated with anti-prion antibody SAF32, followed by incubation of the plates for 2 h at 37° C. The plates were washed and AP-labeled 3F4 antibody was added as the detection antibody. The plates were incubated for about 2 hr at 37° C., washed again, and chemiluminescent AP substrate (LumiphosPlus) was added, incubated for 30 min at 37° C. and read A₄₀₅ by Luminoskan Ascent (Thermo Labsytems). The results are shown in Table 5. The optimum conditions in this experiment for pH dissociation and denaturation were 0.1 N NaOH (pH approx. 13) or phosphoric acid 0.5M (pH approx. 1) for 10 min.

We repeated the Sandwich ELISAs described above with samples having four times as much BH (i.e., having 100 nl vCJD BH or normal BH) spiked into human plasma using pH 13 or pH1 treatment compared to Gdn. These results are shown in Table 6 and are similar to the previous results.

TABLE 5
Sandwich ELISA data for pH and Gdn denatured PrP from pull-down

| | | | | | ELISA Data | | |
|---|---|---|---|---|---|---|---|
| Treatment | pH | Neutralization | Final pH | Sample | Ave | SD | S/B |
| 15 ul GdnSCN 3M | 5.9 | 210 ul H₂O | 6.0 | vCJD BH + | 53.8 | 3.3 | 2.5 |
| | | | | Plasma | | | |
| | | | | Normal BH + | 15.8 | 6.2 | 0.7 |
| | | | | plasma | | | |
| | | | | Plasma only | 21.6 | 5.4 | 1.0 |
| 50 ul NaOH | 10 | 20 ul NaH₂PO₄ | 7 | vCJD BH + | 8.6 | 1.1 | 1.0 |
| 0.0001M | | 0.0003 M | | Plasma | | | |
| | | | | Normal BH + | 8.1 | 1.0 | 1.0 |
| | | | | plasma | | | |
| | | | | Plasma only | 8.3 | 1.0 | 1.0 |
| 50 ul NaOH | 12 | 20 ul NaH₂PO₄ | 7 | vCJD BH + | 6.7 | 0.6 | 1.2 |
| 0.01M | | 0.03 M | | Plasma | | | |
| | | | | Normal BH + | 5.4 | 0.2 | 1.0 |
| | | | | plasma | | | |
| | | | | Plasma only | 5.5 | 0.6 | 1.0 |
| 50 ul NaOH | 13 | 20 ul NaH₂PO₄ | 7 | vCJD BH + | 47.8 | 2.6 | 5.8 |
| 0.1M | | 0.3 M | | Plasma | | | |
| | | | | Normal BH + | 7.1 | 0.9 | 0.9 |
| | | | | plasma | | | |
| | | | | Plasma only | 8.2 | 1.8 | 1.0 |
| 50 ul H₃PO₄ | 4 | 25 ul NaOH | 7 | vCJD BH + | 9.7 | 2.0 | 1.0 |
| 0.00007 M | | 0.0031 N | | Plasma | | | |
| | | | | Normal BH + | 9.2 | 1.3 | 0.9 |
| | | | | plasma | | | |
| | | | | Plasma only | 9.9 | 0.8 | 1.0 |
| 50 ul H₃PO₄ | 2 | 25 ul NaOH | 7 | vCJD BH + | 10.1 | 0.5 | 1.0 |
| 0.12 M | | 0.31 N | | Plasma | | | |
| | | | | Normal BH + | 13.4 | 6.6 | 1.3 |
| | | | | plasma | | | |
| | | | | Plasma only | 10.1 | 1.1 | 1.0 |
| 50 ul H₃PO₄ | 1 | 25 ul NaOH | 7 | vCJD BH + | 44.6 | 12.9 | 4.3 |
| 0.5 M | | 3.1 N | | Plasma | | | |
| | | | | Normal BH + | 10.6 | 2.3 | 1.0 |
| | | | | plasma | | | |
| | | | | Plasma only | 10.4 | 1.6 | 1.0 |

TABLE 6
Sandwich ELISA with 100 nl BH

| Treatment | Sample | Ave LU | SD |
|---|---|---|---|
| High pH (NaOH 0.1 N) | Plasma + vCJD BH | 260.3 | 14.6 |
| | Plasma + Normal BH | 14.9 | 0.9 |
| | Plasma | 12.9 | 0.8 |
| Low pH (H3PO4 0.5 M) | Plasma + vCJD BH | 249.9 | 16.1 |
| | Plasma + Normal BH | 20.5 | 2.5 |
| | Plasma | 16.7 | 2.0 |
| GdnSCN 3M | Plasma + vCJD BH | 252.7 | 13.9 |
| | Plasma + Normal BH | 26.6 | 14.6 |
| | Plasma | 20.5 | 5.7 |

A Sandwich ELISA was carried out similar to that described above but using a different anti-prion antibody as the capture antibody. AP-3F4 was used for detection as described above. 6H4 (commercially available form Prionics AG) was used for capture. Two other anti-prion antibodies, C2 and C17, were also used as capture antibodies. C2 recognizes an epitope at the N-terminal of the prion protein in the octarepeat. C17 recognizes an epitope in the C-terminal region between residues 121-231. Only the high pH treatment was used in this experiment and was carried out for 60 min. followed by neutralization to pH 7 as described above. Treatment with GdnSCN 3 M for 10 min. was used for comparison. The results are shown in Table 7.

TABLE 7
Sandwich ELISA data using 3 different capture antibodies

| | ELISA data | | |
|---|---|---|---|
| | Capture Antibody | | |
| Treatment | C2 | C17 | 6H4 |

| | | | | |
|---|---|---|---|---|
| 0 | N NaOH | 16.8 | 16.3 | 0.6 |
| 0.05 | N NaOH | 433.4 | 535.1 | 137.6 |
| 0.5 | N NaOH | 301.0 | 20.5 | 62.5 |
| 1 | N NaOH | 165.8 | 29.4 | 67.6 |
| 3 | M GdnSCN | 463.2 | 922.1 | 148.6 |

### Example 4

### Surrogate Control Production

### A. Surrogate Recognizes Peptide Reagent

A surrogate control that recognizes peptide reagent QWNKPSKPKTNMKHMGGG (SEQ ID NO:198 with C-terminal GGG linker) is prepared as follows. A 6H4 epitope peptide sequence (DWEDRYYRE, SEQ ID NO:264) is prepared with a terminal cysteine (DWEDRYYREC, SEQ ID NO:265 or CDWEDRYYRE, SEQ ID NO:266) using standard techniques and conjugated to 3F4 antibody using a crosslinking reagent such as Sulfo-SMCC (Sulfosuccinimidal 4-[N-maleimidomethyl]-cyclohexane-1-carboxylate). Extensive dialysis is performed to remove unreacted crosslinker and free peptide. The surrogate control thus prepared can be used in connection with prion detection assays that utilize peptide reagents comprising the sequence “MKHM” (SEQ ID NO:261), for example peptide reagents as depicted in or derived from any of SEQ ID NOs:183, 188, 193, 198, 206, 211, 216, 224, 229, 234, 243 or 244.

### B. Surrogate Recognizing Auxiliary Motif on Peptide Reagent

A surrogate control that binds to peptide reagent GGGKKRPKPGG (SEQ ID NO:14 with N-terminal GGG linker), where the peptide reagent further includes biotin, is prepared as follows. A 6H4 epitope peptide sequence (DWEDRYYRE, SEQ ID NO:264) is prepared with a terminal cysteine (DVVEDRYYREC, SEQ ID NO:265 or CDWEDRYYRE, SEQ ID NO:266) using standard techniques and conjugated to streptavidin using a crosslinking reagent such as Sulfo-SMCC (Sulfosuccinimidal 4-[N-maleimidomethyl]-cyclohexane-1-carboxylate). Extensive dialysis is performed to remove unreacted crosslinker and free peptide.

### C. Two-Peptide Domain Surrogate for Sandwich Assays

A bifunctional surrogate control that recognizes prion-binding reagent 3F4 and primary antibody 6H4 is prepared as follows. A peptide including a 3F4 epitope, a 6H4 epitope and a linker is prepared using standard solid phase peptide synthesis techniques. In particular, MKHMGGGGGDWEDRYYRE (SEQ ID NO:267) is synthesized, where MKHM (SEQ ID NO:261) is an epitope recognized by 3F4, GGGGG (SEQ ID NO:268) is a linker and DWEDRYYRE (SEQ ID NO:264) is an epitope recognized by 6H4.

Although preferred embodiments of the subject invention have been described in some detail, it is understood that obvious variations can be made without departing from the spirit and the scope of the invention as defined herein.

1. A method for detecting the presence of a pathogenic prion in a sample comprising:
(a) providing a first solid support comprising a peptide reagent derived from a peptide having a sequence selected from the group consisting of SEQ ID NO:12-260;
(b) contacting the first solid support with a sample under conditions that allow pathogenic prion proteins, when present in the sample, to bind to the peptide reagent to form a first complex;
(c) removing unbound sample material;
(d) dissociating the pathogenic prion proteins from the first complex; and
(e) detecting the dissociated pathogenic prions using a prion-binding reagent.

2. A method for detecting the presence of a pathogenic prion in a sample comprising:
(a) providing a first solid support comprising a peptide reagent derived from a peptide having a sequence selected from the group consisting of SEQ ID NO:12-260;
(b) contacting the first solid support with a sample under conditions that allow pathogenic prion proteins, when present in the sample, to bind to the peptide reagent to form a first complex;
(c) removing unbound sample material;
(d) dissociating the pathogenic prion proteins from the first complex;
(e) separating the dissociated pathogenic prion proteins from the first solid support;
(f) contacting the dissociated pathogenic prion proteins with a second solid support under conditions that allow the dissociated prion protein to adhere to the second solid support; and
(g) detecting the adhered pathogenic prions on the second solid support using a prion-binding reagent.

3. A method for detecting the presence of a pathogenic prion in a sample comprising:
(a) providing a first solid support comprising a peptide reagent derived from a peptide having a sequence selected from the group consisting of SEQ ID NO:12-260;
(b) contacting the first solid support with a sample under conditions that allow pathogenic prion proteins, when present in the sample, to bind to the peptide reagent to form a first complex;
(c) removing unbound sample material;
(d) dissociating the pathogenic prion proteins from the first complex, whereby the pathogenic prion protein is denatured;
(e) separating the dissociated denatured pathogenic prion proteins from the first solid support;
(f) contacting the dissociated denatured pathogenic prion proteins with a second solid support, wherein the second solid support comprises a first anti-prion antibody, under conditions that allow the dissociated prion protein to bind to the first anti-prion antibody; and
(g) detecting the bound prion proteins on the second solid support with a second anti-prion antibody.

4. The method of any of claims 1, 2 or 3, wherein the dissociating step comprises contacting the bound pathogenic prion protein with a salt or a chaotropic agent.

5. The method of claim 4, wherein the chaotropic agent comprises guanidium thiocyanate (GdnSCN) or guanidinium hydrochloride (GdnHCl).

6. The method of claim 5, wherein the concentration of GdnSCN or GdnHCl is between about 3M and about 6M.

7. The method of any of claims 1, 2 or 3, wherein the dissociating step comprises exposing the bound pathogenic prion protein to high or low pH, whereby the dissociated pathogenic prion protein is denatured.

8. The method of claim 7, wherein the pH is above 12 or below 2.

9. The method of claim 8, wherein the pH is between 12.5 and 13.0.

10. The method of claim 7, wherein the bound pathogenic prion protein is exposed to a high pH by the addition of NaOH to a concentration of 0.05 N to 0.15 N.

11. The method of claim 7, wherein the exposing step is carried out for no more than 15 minutes.

12. The method of claim 11, wherein the exposing step is carried out for no more than 10 minutes.

13. The method of claim 7, further comprising the step of neutralizing the pH of the denatured, dissociated pathogenic prion protein to between 7.0 and 7.5.

14. The method of claim 10, wherein the pH is neutralized by the addition of phosphoric acid or a sodium salt thereof.

15. The method of claim 1, 2 or 3, wherein the first solid support comprises magnetic beads.

16. The method of claim 1 or claim 2, wherein the prion-binding reagent is an anti-prion antibody.

17. The method of claim 1, 2 or 3, wherein the first or second solid support comprises a microtiter plate or a magnetic bead.

18. The method of claim 3, wherein the first or second anti-prion antibody binds to the denatured form of the prion protein.

19. The method of claim 18, wherein one of the first or second anti-prion antibody recognizes an epitope in the amino-terminal of the prion protein.

20. The method of claim 19, wherein one of the first or second anti-prion antibody recognizes an epitope within residues 23-90 of the prion protein.

21. The method of claim 18, wherein the anti-prion antibody is selected from the group consisting of Fab D 18, 3F4, SAF-32, 6H4.

22. The method of claim 1, 2 or 3, wherein the peptide reagent is derived from a peptide having a sequence selected from the group consisting of one or more of SEQ ID NOs:66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 133, 134, 135, 136, 56, 57, 65, 82, and 84.

23. The method of claim 22, wherein the peptide reagent is derived from a peptide having a sequence selected from the group consisting of one or more of SEQ ID NOs: 66, 67, 68, 72, 81, 96, 97, 98, 107, 108, 119, 120, 121, 122, 123, 124, 125, 126, 127, 134 and 135.

24. The method of claim 22, wherein the peptide reagent is derived from a peptide having a sequence selected from the group consisting of one or more of SEQ ID NOs: 14, 35, 36, 37, 40, 50, 51, 77, 89, 100, 101, 109, 110, 111, 112, 113, 114, 115, 116, 117, 118, 129, 130, 131, 132, 133 or 128.

25. The method of claim 22, wherein the peptide reagent is derived from a peptide having a sequence selected from the group consisting of one or more of SEQ ID NOs: 56, 57, 65, 82, 84, and 136.

26. The method of claim 22, wherein the peptide reagent is derived from a peptide having a sequence selected from the group consisting of one or more of SEQ ID NOs: 14, 51, 117, 118, 111, 114, 131, 132, 133, 68, 122, 81, 134, 135, 57, 136 and 85.

27. The method of claim 22, wherein the peptide reagent comprises SEQ ID NO:14.

28. The method of claim 22, wherein the peptide reagent comprises SEQ ID NO:51.

29. The method of claim 22, wherein the peptide reagent comprises SEQ ID NO:68.

30. A surrogate control for use in a prion detection assay, the surrogate control comprising: a first surrogate domain that binds to a peptide reagent derived from a peptide having a sequence selected from the group consisting of SEQ ID NO: 12-260; and a second surrogate domain that binds to a detection reagent used in the prion assay, wherein said prion detection assay utilizes a peptide reagent and a detection reagent to detect the presence of a pathogenic prion protein in a sample.

31. A method for detecting the presence of a pathogenic prion in a sample comprising: (a) contacting, in a test container, a sample suspected of containing a pathogenic prion with a
first peptide reagent that interacts preferentially with a pathogenic prion protein, under conditions that allow the binding of the first peptide reagent to the pathogenic prion protein, if present, to form a first complex; (b) contacting, in a control container, the first peptide reagent with the surrogate control of claim 30, under conditions that allow the binding of the surrogate control to the first peptide reagent; (c) detecting the presence the pathogenic prion, if any, in the sample by its binding to the first peptide reagent; and (d) confirming the presence of the detected pathogenic prion by detecting the presence of surrogate control bound to the first peptide reagent.
